(19) United States
(12) Patent Application Publication
(10) Pub. No.: US 2025/0136596 A1
(43) Pub. Date: May 1, 2025

(54) MEMBRANE-ASSOCIATED TYROSINE- AND THREONINE-SPECIFIC CDC2-INHIBITORY KINASE (PKMYT1) INHIBITORS AND USES THEREOF

(71) Applicant: Insilico Medicine IP Limited, Hong Kong (CN)

(72) Inventors: Xiao DING, Shanghai (CN); Yangguang LI, Shanghai (CN); Yingtao LIU, Shanghai (CN); Fanye MENG, Shanghai (CN); Feng REN, Shanghai (CN); Yazhou WANG, Shanghai (CN); Min ZHENG, Shanghai (CN)

(21) Appl. No.: 18/837,846

(22) PCT Filed: Feb. 17, 2023
(86) PCT No.: PCT/CN2023/076723

§ 371 Date: Aug. 12, 2024

(30) Foreign Application Priority Data
Feb. 18, 2022 (WO) PCT/CN2022/076941

(51) Int. Cl. C07D 471/04; A61K 31/444; A61K 31/4545; A61K 31/496; A61K 31/497; A61K 31/4985; A61K 31/501; A61K 31/506; A61K 31/53; A61K 31/541; C07D 487/04; C07D 519/00

(52) U.S. Cl. CPC C07D 471/04; A61K 31/444; A61K 31/4545; A61K 31/496; A61K 31/497; A61K 31/4985; A61K 31/501; A61K 31/506; A61K 31/53; A61K 31/541; C07D 487/04; C07D 519/00

(57) ABSTRACT

Described herein are PKMYT1 (Myt1) inhibitors and pharmaceutical compositions comprising said inhibitors. The subject compounds and compositions are useful for the treatment of a disease or disorder associated with PKMYT1.

### CROSS-REFERENCE

This patent application claims the benefit of International Application No. PCT/CN2022/076941, filed Feb. 18, 2022; which is incorporated herein by reference in its entirety.

### BACKGROUND

PKMYT1 (or Myt1) is a member of the Wee family and was first reported as a kinase capable of phosphorylating Cdc2 efficiently on both threonine-14 and tyrosine-15 in a Xenopus frog. PKMYT1 inhibits cell cycle progression by inhibiting the activities of cell cycle-associated proteins, such as Cyclin A, CDK1, and CDK2. PKMYT1 also drives the progression of a variety of tumors.

The inhibitory phosphorylation of cdc2 is important for the timing of entry into mitosis. Entry into mitosis is initiated by the M phase-promoting factor (MPF), a complex containing the cdc2 protein kinase and cyclin B. Proper regulation of MPF ensures that mitosis occurs only after earlier phases of the cell cycle are complete. Phosphorylation of cdc2 at Tyr-15 and Thr-14 suppresses this activity during interphase (G1, S, and G2). At G2-M transition, cdc2 is dephosphorylated at Tyr-15 and Thr-14 allowing MPF to phosphorylate its mitotic substrates.

Studies have shown that premature activation of cdc2 leads to mitotic catastrophe and cell death. Inhibition of Myt1 is predicted to cause premature activation of cdc2, and thus would kill rapidly proliferating cells. In addition, Myt1 inhibition is predicted to reduce resistance to conventional DNA-damaging chemotherapeutics, because the mechanisms by which cells avoid death involve arrest in the G2 phase of the cell cycle, and repair or DNA damage prior to division. That arrest should be prevented by blocking Myt1 inhibitory phosphorylation of cdc2. Thus forcing the cell to enter mitosis prematurely. Myt1 kinase is an important cell cycle regulator, particularly at the G2/M phase. This is due to cell cycle regulation and subsequent repair of damage to DNA or mitotic apparatus, the targets for most effective chemotherapeutic agents. Myt1 kinase offers a point of intervention downstream from these mechanisms by which tumor cells develop resistance. Inhibition of Myt1 could in and of itself have therapeutic benefit in reducing tumor proliferation, and in addition, could be used in conjunction with conventional chemotherapies to overcome drug resistance.

Based on the foregoing, there is a need to identify a potent PKMYT1 (Myt1) kinase inhibitor for the treatment of cancer.

### SUMMARY

Disclosed herein is a compound of Formula (I), or a pharmaceutically acceptable salt thereof:

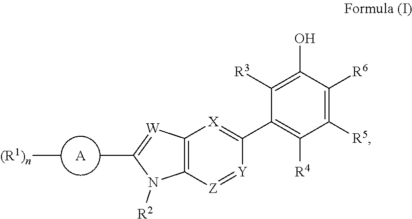

wherein:
Ring A is cycloalkyl, heterocycloalkyl, aryl, or heteroaryl;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(=O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more Ria;
or two R¹ on the same atom are taken together to form an oxo;
each R^{1a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(=O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{1a} on the same atom are taken together to form an oxo;
n is 0-6;
R² is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
W is N or CR^{W};
R^{W} is hydrogen, halogen, —CN, —OH, —OR^{a}, —SH, —SR^{a}, —NR^{c}R^{d}, —C(═O)R^{a}, —C(═O)ORW, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Wa};
each R^{Wa} is independently halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Wa} on the same atom are taken together to form an oxo;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R³ is halogen, C₁-C₆alkyl, C₁-C₆haloalkyl, C₁-C₆hydroxyalkyl, C₁-C₅aminoalkyl, or C₁-C₆heteroalkyl, cycloalkyl, or heterocycloalkyl;
R⁴ is halogen, C₁-C₆alkyl, C₁-C₆haloalkyl, C₁-C₆hydroxyalkyl, C₁-C₅aminoalkyl, or C₁-C₆heteroalkyl, cycloalkyl, or heterocycloalkyl;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

In some embodiments of a compound of Formula (I), the compound is of Formula (Ia):

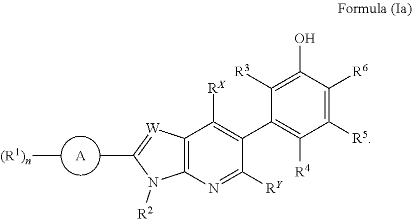

In some embodiments of a compound of Formula (I), the compound is of Formula (Ib):

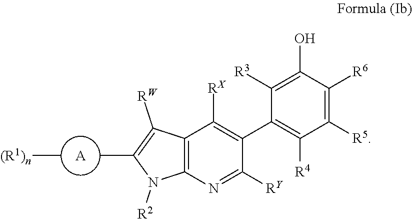

In some embodiments of a compound of Formula (I), the compound is of Formula (Ic):

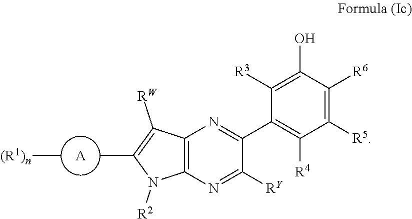

Also disclosed herein is a pharmaceutical composition comprising a compound disclosed herein, or a pharmaceutically acceptable salt thereof, and a pharmaceutically acceptable excipient.

Also disclosed herein is a method of treating cancer in a subject in need thereof, the method comprising administering to the subject a compound disclosed herein, or a pharmaceutically acceptable salt thereof.

Also disclosed herein is a method of modulating PKMYT1 in a subject, the method comprising administering to the subject the compound disclosed herein, or a pharmaceutically acceptable salt thereof.

Also disclosed herein is a method of inhibiting PKMYT1 in a subject, the method comprising administering to the subject the compound disclosed herein, or a pharmaceutically acceptable salt thereof.

Also disclosed herein is method of inhibiting PKMYT1 and WEE1 in a subject, the method comprising administering to the subject the compound disclosed herein, or a pharmaceutically acceptable salt thereof.

In some embodiments, the subject has cancer. In some embodiments, the cancer depends on the activity of PKMYT1. In some embodiments, the cancer overexpresses CCNE1. In some embodiments, the cancer has an inactivating mutation in the FBXW7 gene. In some embodiments, the cancer is a solid tumor. In some embodiments, the cancer is breast cancer, colorectal cancer, endometrial cancer, esophageal cancer, glioblastoma, hepatocellular carcinoma, lung cancer, neuroblastoma, ovarian cancer, prostate cancer, stomach cancer, or uterine cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to =O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)=CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5- to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₇ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

The terms “treat,” “treating” or “treatment,” as used herein, include alleviating, abating, or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

As used herein, a “disease or disorder associated with PKMYT1” or, alternatively, “a PKMYT1-mediated disease or disorder” means any disease or other deleterious condition in which PKMYT1, or a mutant thereof, is known or suspected to play a role.

As used herein, a “disease or disorder associated with Myt1” or, alternatively, “a Myt1-mediated disease or disorder” means any disease or other deleterious condition in which Myt1, or a mutant thereof, is known or suspected to play a role.

### Compounds

Described herein are compounds, or a pharmaceutically acceptable salt thereof useful in the treatment of a disease or disorder associated with PKMYT1.

Disclosed herein is a compound of Formula (I), or a pharmaceutically acceptable salt thereof:

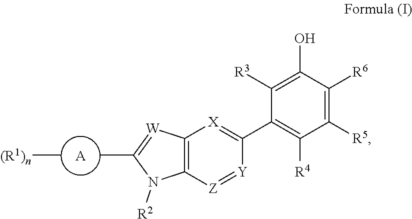

wherein:
Ring A is cycloalkyl, heterocycloalkyl, aryl, or heteroaryl;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(=O)₂R^{a}, —C(═O)R^{a}, —C(═O)OR^{b}, —C(═O)NR^{c}R^{d}, C₁-C₆alkyl, C₁-C₅haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{1a};
or two R¹ on the same atom are taken together to form an oxo;
each R^{1a} is independently halogen, —CN, —NO₂, —OH, —OR, —OC(O)R^{a}, —OC(═O)OR^{b}, —OC(═O)NR^{c}R^{d}, —SH, —SR^{a}, —S(═O)R^{a}, —S(═O)₂R^{a}—S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{1a} on the same atom are taken together to form an oxo;
n is 0-6;
R² is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
W is N or CR^{W};
R^{W} is hydrogen, halogen, —CN, —OH, —OR^{a}, —SH, —SR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Wa};
each R^{Wa} is independently halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Wa} on the same atom are taken together to form an oxo;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R³ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁴ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

Disclosed herein is a compound of Formula (I), or a pharmaceutically acceptable salt thereof:

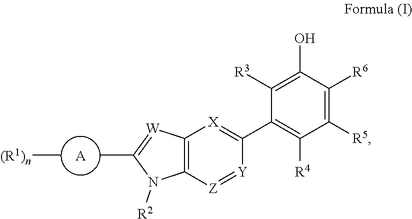

wherein:
Ring A is cycloalkyl, heterocycloalkyl, aryl, or heteroaryl;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(=O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{1a};
or two R¹ on the same atom are taken together to form an oxo;
each R^{1a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(=O)₂R^{a}, —C(═O)R^{a}, —C(═O)OR^{b}, —C(═O)NR^{c}R^{d}, C₁-C₆alkyl, C₁-C₅haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{1a} on the same atom are taken together to form an oxo;
n is 0-6;
R² is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
W is N or CR^{W};
R^{W} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₅haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R³ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁴ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆ aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

In some embodiments of a compound of Formula (I), X is N. In some embodiments of a compound of Formula (I), X is CR^{X}.

In some embodiments of a compound of Formula (I), Y is N. In some embodiments of a compound of Formula (I), Y is CR^{Y}.

In some embodiments of a compound of Formula (I), W is N. In some embodiments of a compound of Formula (I), W is CR^{W}.

In some embodiments of a compound of Formula (I), Z is N. In some embodiments of a compound of Formula (I), Z is CR^{Z}.

In some embodiments of a compound of Formula (I), the compound is of Formula (Ia):

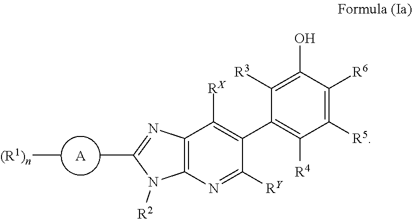

In some embodiments of a compound of Formula (I), the compound is of Formula (Ib):

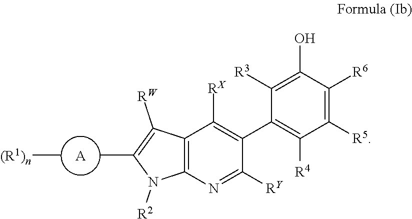

In some embodiments of a compound of Formula (I), the compound is of Formula (Ic):

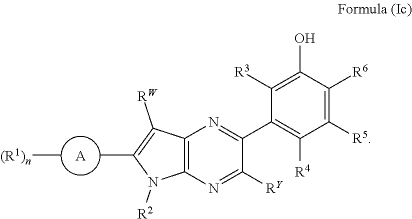

In some embodiments of a compound of Formula (I) or (Ia)-(Ic), R³ is halogen, C₁-C₆alkyl, or C₁-C₆haloalkyl. In some embodiments of a compound of Formula (I) or (Ia)-(Ic), R³ is halogen or C₁-C₆alkyl. In some embodiments of a compound of Formula (I) or (Ia)-(Ic), R³ is C₁-C₆alkyl. In some embodiments of a compound of Formula (I) or (Ia)-(Ic), R³ is C₁-C₃alkyl. In some embodiments of a compound of Formula (I) or (Ia)-(Ic), R³ is n-propyl or isopropyl. In some embodiments of a compound of Formula (I) or (Ia)-(Ic), R³ is ethyl. In some embodiments of a compound of Formula (I) or (Ia)-(Ic), R³ is methyl.

In some embodiments of a compound of Formula (I) or (Ia)-(Ic), R⁴ is halogen, C₁-C₆alkyl, or C₁-C₆haloalkyl. In some embodiments of a compound of Formula (I) or (Ia)-(Ic), R⁴ is halogen or C₁-C₆alkyl. In some embodiments of a compound of Formula (I) or (Ia)-(Ic), R⁴ is C₁-C₆alkyl. In some embodiments of a compound of Formula (I) or (Ia)-(Ic), R⁴ is C₁-C₃alkyl. In some embodiments of a compound of Formula (I) or (Ia)-(Ic), R⁴ is n-propyl or isopropyl. In some embodiments of a compound of Formula (I) or (Ia)-(Ic), R⁴ is ethyl. In some embodiments of a compound of Formula (I) or (Ia)-(Ic), R⁴ is methyl.

In some embodiments of a compound of Formula (I) or (Ia)-(Ic), R⁵ is hydrogen, halogen, C₁-C₆alkyl, or C₁-C₆haloalkyl. In some embodiments of a compound of Formula (I) or (Ia)-(Ic), R⁵ is hydrogen, halogen, C₁-C₃alkyl, or C₁-C₃haloalkyl. In some embodiments of a compound of Formula (I) or (Ia)-(Ic), R⁵ is hydrogen.

In some embodiments of a compound of Formula (I) or (Ia)-(Ic), R⁶ is hydrogen, halogen, C₁-C₆alkyl, or C₁-C₆haloalkyl. In some embodiments of a compound of Formula (I) or (Ia)-(Ic), R⁶ is hydrogen, halogen, C₁-C₃alkyl, or C₁-C₃haloalkyl. In some embodiments of a compound of Formula (I) or (Ia)-(Ic), R⁶ is hydrogen.

In some embodiments of a compound of Formula (I) or (Ia)-(Ic), R² is hydrogen or C₁-C₆alkyl. In some embodiments of a compound of Formula (I) or (Ia)-(Ic), R² is hydrogen.

In some embodiments of a compound of Formula (I) or (Ia)-(Ib), R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (I) or (Ia)-(Ib), R^{X} is hydrogen, halogen, —CN, —C(═O)NR^{c}R^{d}, C₁-C₆alkyl, or C₁-C₆haloalkyl.

In some embodiments of a compound of Formula (I) or (Ia)-(Ib), R^{X} is hydrogen, halogen, —CN, C₁-C₆alkyl, or C₁-C₆haloalkyl.

In some embodiments of a compound of Formula (I) or (Ia)-(Ib), R^{X} is hydrogen, halogen, or —CN.

In some embodiments of a compound of Formula (I) or (Ia)-(Ib), R^{X} is hydrogen, halogen, C₁-C₆alkyl, or C₁-C₆haloalkyl.

In some embodiments of a compound of Formula (I) or (Ia)-(Ib), R^{X} is hydrogen, halogen, or C₁-C₆alkyl. In some embodiments of a compound of Formula (I) or (Ia)-(Ib), R^{X} is hydrogen or halogen. In some embodiments of a compound of Formula (I) or (Ia)-(Ib), R^{X} is hydrogen. In some embodiments of a compound of Formula (I) or (Ia)-(Ib), R^{X} is halogen. In some embodiments of a compound of Formula (T) or (Ia)-(Ib), R^{X} is C₁-C₆alkyl. In some embodiments of a compound of Formula (I) or (Ia)-(Ib), R^{X} is C₁-C₃alkyl. In some embodiments of a compound of Formula (T) or (1a)-(Ib), R^{X} is —CN.

In some embodiments of a compound of Formula (I) or (Ia)-(Ic), R¹ is hydrogen, halogen, C₁-C₆alkyl, or C₁-C₆haloalkyl. In some embodiments of a compound of Formula (T) or (Ia)-(Ic), R^{Y} is hydrogen, halogen, C₁-C₃alkyl, or C₁-C₃haloalkyl. In some embodiments of a compound of Formula (I) or (Ia)-(Ic), R^{Y} is hydrogen.

In some embodiments of a compound of Formula (I), R^{Z} is hydrogen, halogen, or C₁-C₆alkyl. In some embodiments of a compound of Formula (I), R^{Z} is hydrogen or halogen. In some embodiments of a compound of Formula (I), R^{Z} is hydrogen.

In some embodiments of a compound of Formula (I) or (Ib)-(Ic), R^{W} is hydrogen, halogen, —CN, —OH, —OR^{a}, —SH, —SR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkynyl, cycloalkyl, or heterocycloalkyl; wherein the alkyl, alkynyl, cycloalkyl, and heterocycloalkyl is optionally substituted with one or more R^{Wa}.

In some embodiments of a compound of Formula (I) or (Ib)-(Ic), R^{W} is hydrogen, halogen, —CN, —OH, —OR^{a}, —SR^{a}, C₁-C₆alkyl, C₁-C₆haloalkyl, C₂-C₆alkynyl, cycloalkyl, or heterocycloalkyl; wherein the alkyl, alkynyl, cycloalkyl, and heterocycloalkyl is optionally substituted with one or more R^{Wa}.

In some embodiments of a compound of Formula (I) or (Ib)-(Ic), R^{W} is hydrogen, halogen, —OR^{a}, —SR^{a}, C₁-C₆alkyl, C₁-C₆haloalkyl, C₂-C₆alkynyl, cycloalkyl, or heterocycloalkyl; wherein the alkyl, alkynyl, cycloalkyl, and heterocycloalkyl is optionally substituted with one or more R^{Wa}.

In some embodiments of a compound of Formula (I) or (Ib)-(Ic), R^{W} is C₂-C₆alkynyl optionally substituted with one or more R^{Wa}.

In some embodiments of a compound of Formula (I) or (Ib)-(Ic), R^{W} is hydrogen, halogen, —OH, —OR^{a}, —NR^{c}R^{d}, C₁-C₆alkyl, C₁-C₆haloalkyl, cycloalkyl, heterocycloalkyl, aryl, or heteroaryl; wherein the alkyl, cycloalkyl, heterocycloalkyl, aryl, and heteroaryl is optionally substituted with one or more R.

In some embodiments of a compound of Formula (I) or (Ib)-(Ic), R^{W} is hydrogen, halogen, —OR^{a}, C₁-C₆alkyl, C₁-C₆haloalkyl, cycloalkyl, or heterocycloalkyl; wherein the alkyl, cycloalkyl, and heterocycloalkyl is optionally substituted with one or more R. In some embodiments of a compound of Formula (I) or (Ib)-(Ic), R^{W} is hydrogen, —OR^{a}, C₁-C₆alkyl, or heterocycloalkyl; wherein the alkyl and heterocycloalkyl is optionally substituted with one or more R. In some embodiments of a compound of Formula (I) or (Ib)-(Ic), R^{W} is cycloalkyl, heterocycloalkyl, aryl, or heteroaryl, each of which is optionally substituted with one or more R. In some embodiments of a compound of Formula (I) or (Ib)-(Ic), R^{W} is cycloalkyl or heterocycloalkyl, each of which is optionally substituted with one or more R. In some embodiments, R^{W} is 5 membered ring. In some embodiments, R^{W} is 6 membered ring. In some embodiments, R^{W} is

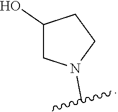 [SMILES: *N1CCC(O)C1]

In some embodiments, R^{W} is

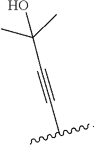 [SMILES: *C#CC(C)(C)O]

In some embodiments, R^{W} is

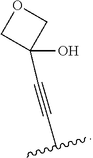 [SMILES: *C#CC1(O)COC1]

In some embodiments of a compound of Formula (I) or (Ib)-(Ic), R^{W} is hydrogen. In some embodiments of a compound of Formula (I) or (Ib)-(Ic), R^{W} is halogen. In some embodiments of a compound of Formula (I) or (Ib)-(Ic), R^{W} is chloro. In some embodiments of a compound of Formula (I) or (Ib)-(Ic), R^{W} is heterocycloalkyl optionally substituted with one or more R. In some embodiments of a compound of Formula (I) or (Ib)-(Ic), R^{W} is —OR^{a}. In some embodiments of a compound of Formula (I) or (Ib)-(Ic), R^{W} is —OR^{a}, and R^{a} is cycloalkyl, heterocycloalkyl, aryl, or heteroaryl, each of which is optionally substituted with one or more R. In some embodiments, R^{a} is 5 membered ring. In some embodiments, R^{a} is 5- or 6-membered cycloalkyl or heterocycloalkyl. In some embodiments, R^{a} is 6 membered ring. In some embodiments, R^{a} is 5- or 6-membered aryl or heteroaryl. In some embodiments of a compound of Formula (I) or (Ib)-(Ic), R^{W} is

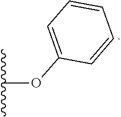 [SMILES: *Oc1ccccc1]

In some embodiments of a compound of Formula (I) or (Ib)-(Ic), R^{W} is

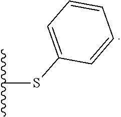 [SMILES: *Sc1ccccc1]

In some embodiments of a compound of Formula (I) or (Ib)-(Ic), R^{W} is C₁-C₆alkyl. In some embodiments of a compound of Formula (I) or (Ib)-(Ic), R^{W} is C₁-C₃alkyl. In some embodiments of a compound of Formula (I) or (Ib)-(Ic), R^{W} is C₁-C₆haloalkyl. In some embodiments of a compound of Formula (I) or (Ib)-(Ic), R^{W} is C₁-C₃haloalkyl.

In some embodiments of a compound of Formula (I) or (Ib)-(Ic), each R^{Wa} is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R.

In some embodiments of a compound of Formula (I) or (Ib)-(Ic), each R^{Wa} is independently halogen, —CN, —OH, —OR^{a}, —NR^{c}R^{d}, C₁-C₆alkyl, C₁-C₆haloalkyl, cycloalkyl, or heterocycloalkyl; wherein each alkyl, cycloalkyl, and heterocycloalkyl is independently optionally substituted with one or more R.

In some embodiments of a compound of Formula (I) or (Ib)-(Ic), each R^{Wa} is independently halogen, C₁-C₆alkyl, C₁-C₆haloalkyl, cycloalkyl, or heterocycloalkyl; wherein each alkyl, cycloalkyl, and heterocycloalkyl is independently optionally substituted with one or more R.

In some embodiments of a compound of Formula (I) or (Ia)-(Ic), Ring A is cycloalkyl or heterocycloalkyl. In some embodiments of a compound of Formula (I) or (Ia)-(Ic), Ring A is heterocycloalkyl. In some embodiments of a compound of Formula (I) or (Ia)-(Ic), Ring A is 5- or 6-membered heterocycloalkyl. In some embodiments of a compound of Formula (I) or (Ia)-(Ic), Ring A is 5-membered heterocycloalkyl. In some embodiments of a compound of Formula (I) or (Ia)-(Ic), Ring A is 6-membered heterocycloalkyl. In some embodiments of a compound of Formula (I) or (Ia)-(Ic), Ring A is tetrahydropyridinyl.

In some embodiments of a compound of Formula (I) or (Ia)-(Ic), Ring A is aryl or heteroaryl. In some embodiments of a compound of Formula (I) or (Ia)-(Ic), Ring A is heteroaryl.

In some embodiments of a compound of Formula (I) or (Ia)-(Ic), Ring A is 5- or 6-membered heteroaryl. In some embodiments of a compound of Formula (I) or (Ia)-(Ic), Ring A is 5-membered heteroaryl. In some embodiments of a compound of Formula (I) or (Ia)-(Ic), Ring A is 6-membered heteroaryl. In some embodiments of a compound of Formula (I) or (Ia)-(Ic), Ring A is monocyclic heteroaryl. In some embodiments of a compound of Formula (I) or (Ia)-(Ic), Ring A is bicyclic heteroaryl. In some embodiments of a compound of Formula (I) or (Ia)-(Ic), Ring A is 5-6, 6-5, 5-5, or 6-6 fused bicyclic heteroaryl. In some embodiments of a compound of Formula (I) or (Ia)-(Ic), Ring A is fused bicyclic heteroaryl. In some embodiments of a compound of Formula (I) or (Ia)-(Ic), Ring A is pyridinyl. In some embodiments of a compound of Formula (I) or (Ia)-(Ic), Ring A is pyrimidyl. In some embodiments of a compound of Formula (I) or (Ia)-(Ic), Ring A is pyrazinyl. In some embodiments of a compound of Formula (I) or (Ia)-(Ic), Ring A is

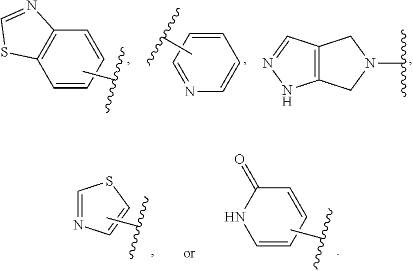

In some embodiments of a compound of Formula (I) or (Ia)-(Ic), Ring A is

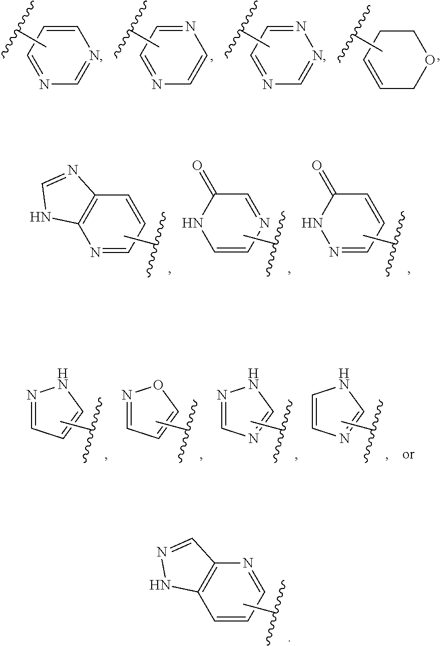

In some embodiments of a compound of Formula (I) or (Ia)-(Ic), Ring A is

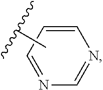

In some embodiments of a compound of Formula (I) or (Ia)-(Ic), each R¹ is independently halogen, —CN, —OH, —OR^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{1a}; or two R¹ on the same atom are taken together to form an oxo.

In some embodiments of a compound of Formula (I) or (Ia)-(Ic), each R¹ is independently halogen, —CN, —OH, —OR^{a}, —S(═O)₂R^{a}, —NR^{c}R^{d}, —C(═O)R^{a}, C₁-C₆alkyl, C₁-C₆haloalkyl, C₁-C₆hydroxyalkyl, C₁-C₆aminoalkyl, C₁-C₆heteroalkyl, C₂-C₆alkynyl, cycloalkyl, heterocycloalkyl, aryl, or heteroaryl; wherein each alkyl, alkynyl, cycloalkyl, heterocycloalkyl, aryl, and heteroaryl is independently optionally substituted with one or more R^{1a}; or two R¹ on the same atom are taken together to form an oxo.

In some embodiments of a compound of Formula (I) or (Ia)-(Ic), each R¹ is independently halogen, —CN, —OH, —OR^{a}, —S(O)₂R^{a}, —NR^{c}R^{d}, —C(═O)R^{a}, C₁-C₆alkyl, C₁-C₆haloalkyl, C₁-C₆hydroxyalkyl, C₁-C₆heteroalkyl, C₂-C₆alkynyl, cycloalkyl, heterocycloalkyl, or heteroaryl; wherein each alkyl, alkynyl, cycloalkyl, heterocycloalkyl, and heteroaryl is independently optionally substituted with one or more R^{1a}; or two R¹ on the same atom are taken together to form an oxo.

In some embodiments of a compound of Formula (I) or (Ia)-(Ic), each R¹ is independently halogen, —CN, —OH, —OR^{a}, —S(O)₂R^{a}, —NR^{c}R^{d}, —C(═O)R^{a}, C₁-C₆alkyl, C₁-C₆haloalkyl, C₁-C₆hydroxyalkyl, C₁-C₆heteroalkyl, or C₂-C₆alkynyl; wherein each alkyl and alkynyl is independently optionally substituted with one or more R^{1a}; or two R¹ on the same atom are taken together to form an oxo.

In some embodiments of a compound of Formula (I) or (Ia)-(Ic), each R¹ is independently cycloalkyl, heterocycloalkyl, or heteroaryl; wherein each cycloalkyl, heterocycloalkyl, and heteroaryl is independently optionally substituted with one or more R^{1a}; or two R¹ on the same atom are taken together to form an oxo.

In some embodiments of a compound of Formula (I) or (Ia)-(Ic), each R¹ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently optionally independently substituted with one or more R^{1a}. In some embodiments, one or more R¹ is —OR^{a}. In some embodiments, one or more R¹ is —NR^{c}R^{d}. In some embodiments, one or more R¹ is —C(═O)R^{a}. In some embodiments, one or more R¹ is optionally substituted C₁-C₆alkyl. In some embodiments, one or more R¹ is optionally substituted C₁-C₆haloalkyl. In some embodiments, one or more R¹ is optionally substituted C₁-C₆hydroxyalkyl. In some embodiments, one or more R¹ is optionally substituted cycloalkyl. In some embodiments, one or more R¹ is optionally substituted heterocycloalkyl. In some embodiments, one or more R¹ is an optionally substituted 6-membered heterocycloalkyl containing 1-2 nitrogen.

In some embodiments of a compound of Formula (I) or (Ia)-(Ic), each R¹ is independently halogen, —OR^{a}, —NR^{c}R^{d}, —C(═O)R^{a}, C₁-C₆alkyl, C₁-C₆haloalkyl, C₁-C₆hydroxyalkyl, cycloalkyl, or heterocycloalkyl; wherein each alkyl, cycloalkyl, and heterocycloalkyl is independently optionally independently substituted with one or more R^{1a}.

In some embodiments of a compound of Formula (I) or (Ia)-(Ic), each R¹ is independently halogen, —OR^{a}, —NR^{c}R^{d}, —C(═O)R^{a}, C₁-C₆alkyl, C₁-C₆hydroxyalkyl, or heterocycloalkyl; wherein each alkyl, and heterocycloalkyl is independently optionally independently substituted with one or more R^{1a}.

In some embodiments of a compound of Formula (I) or (Ia)-(Ic), each R^{1a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, or heterocycloalkyl is independently optionally substituted with one or more R.

In some embodiments of a compound of Formula (I) or (Ia)-(Ic), each R^{1a} is independently halogen, —OH, —C(═O)OR^{b}, —C(═O)NR^{c}R^{d}, C₁-C₆alkyl, C₁-C₆haloalkyl, C₁-C₆hydroxyalkyl, cycloalkyl, or heterocycloalkyl; wherein each alkyl, cycloalkyl, or heterocycloalkyl is independently optionally substituted with one or more R.

In some embodiments of a compound of Formula (I) or (Ia)-(Ic), each R^{1a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, cycloalkyl, or heterocycloalkyl; wherein each alkyl, cycloalkyl, heterocycloalkyl is independently optionally substituted with one or more R. In some embodiments of a compound of Formula (I) or (Ia)-(Ic), each R^{1a} is independently halogen, C₁-C₆alkyl, or C₁-C₆haloalkyl; wherein each alkyl is independently optionally substituted with one or more R. In some embodiments of a compound of Formula (I) or (Ia)-(Ic), each R^{1a} is independently halogen, C₁-C₆alkyl, or C₁-C₆haloalkyl.

In some embodiments of a compound of Formula (I) or (Ia)-(Ic), n is 0-2. In some embodiments of a compound of Formula (I) or (Ia)-(Ic), n is 1 or 2. In some embodiments of a compound of Formula (I) or (Ia)-(Ic), n is 0. In some embodiments of a compound of Formula (I) or (Ia)-(Ic), n is 1. In some embodiments of a compound of Formula (I) or (Ia)-(Ic), n is 2.

In some embodiments of a compound of Formula (I) or (Ia)-(Ic), n is 0.

In some embodiments of a compound of Formula (I) or (Ia)-(Ic),

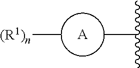

is

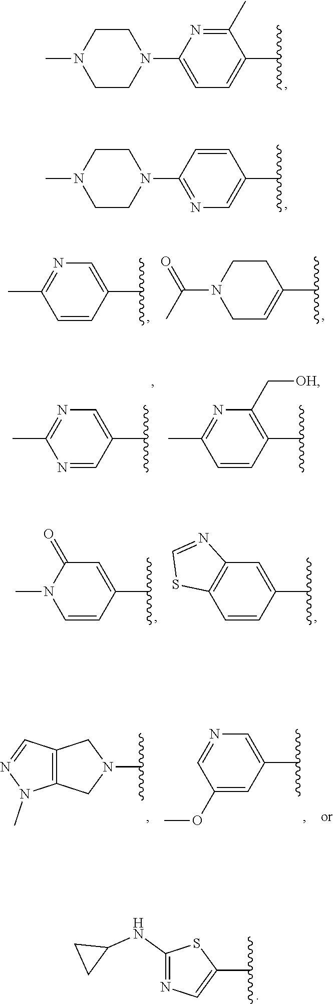

In some embodiments of a compound of Formula (I) or (Ia)-(Ic),

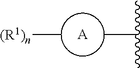

is

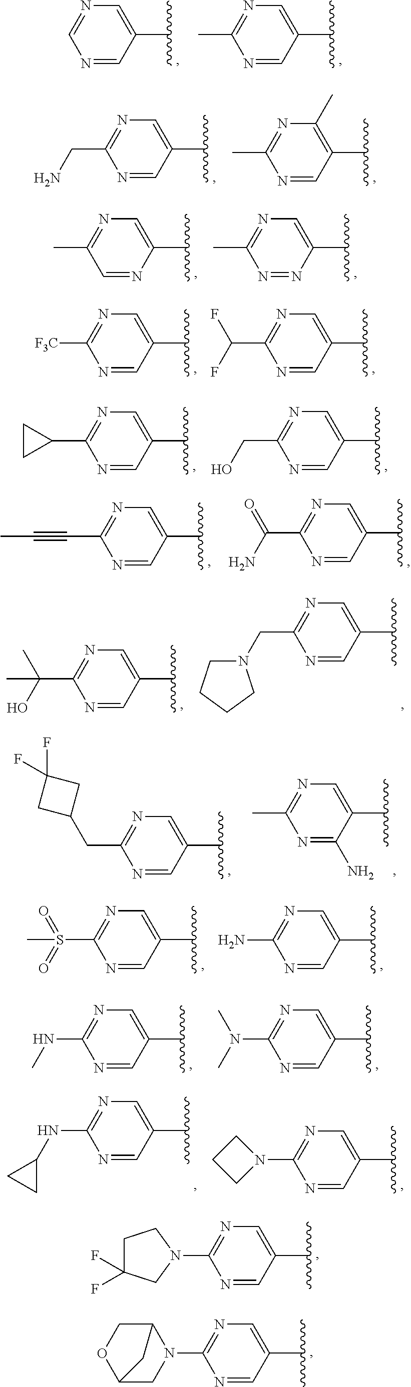

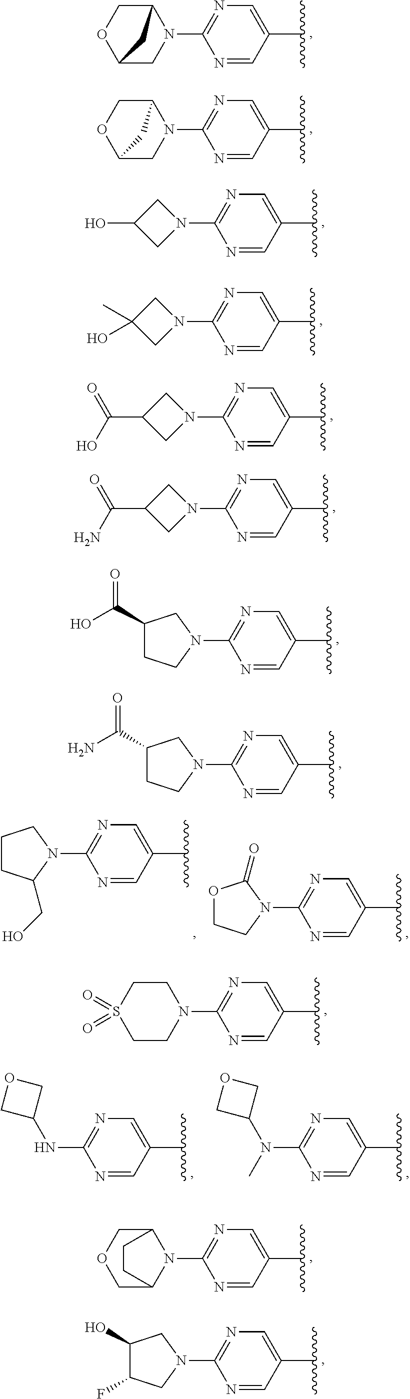

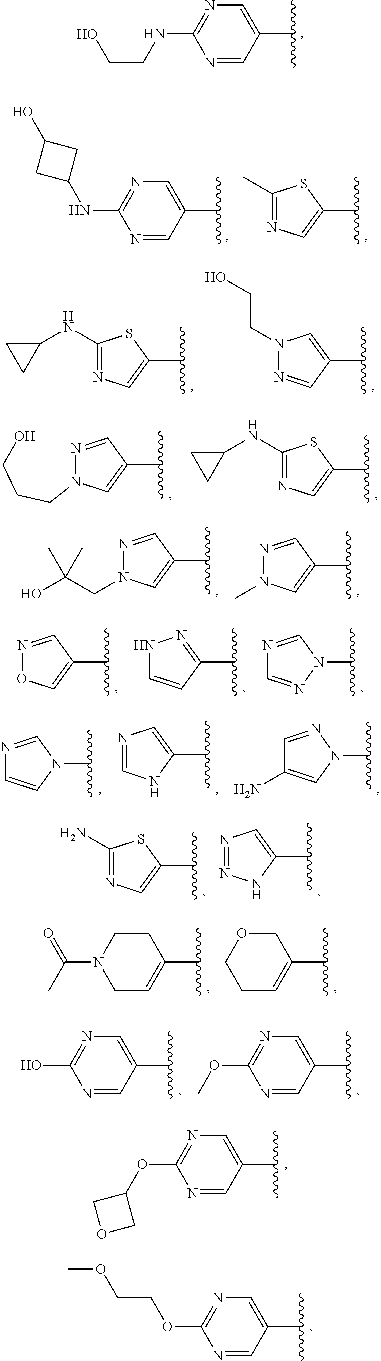

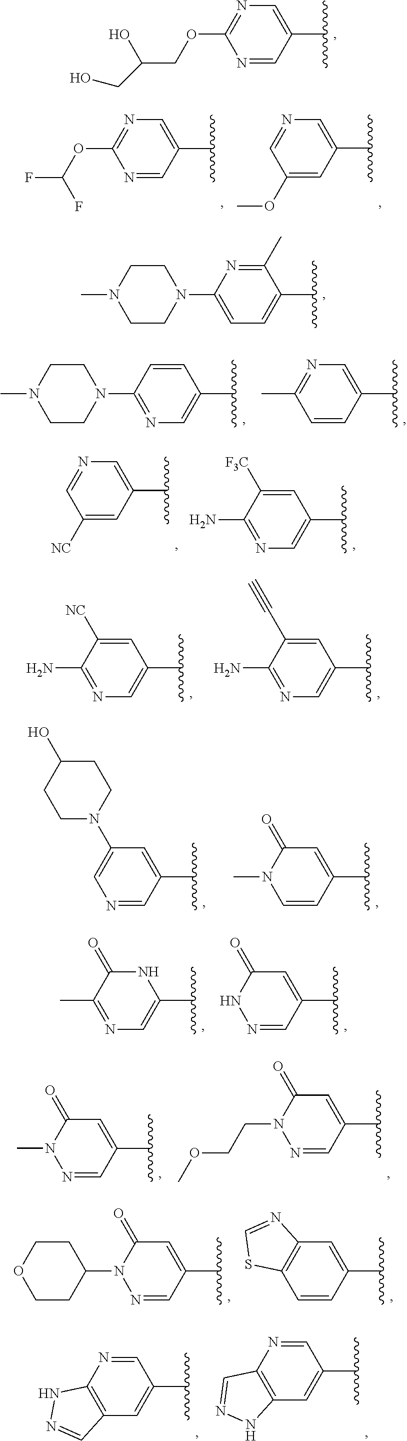

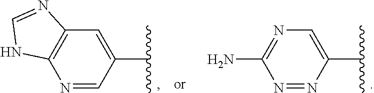 [SMILES: *c1cnc2[nH]cnc2c1 ; *c1cnc(N)nn1]

In some embodiments of a compound of Formula (I) or (Ia) (Ic),

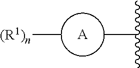

its

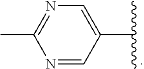 [SMILES: *c1cnc(C)nc1]

In some embodiments of a compound of Formula (I) or (Ia)-(Ic),

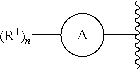

is

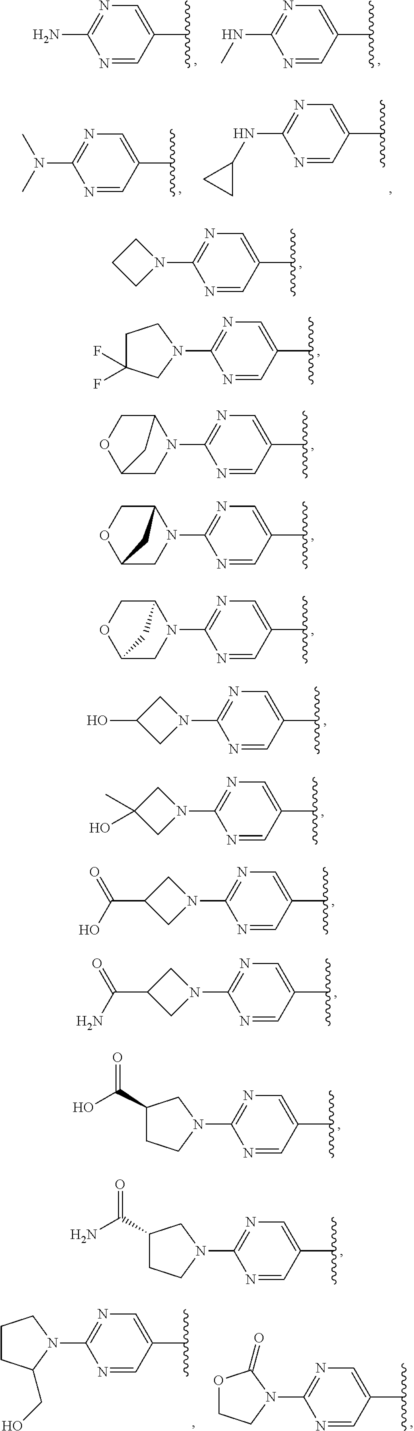

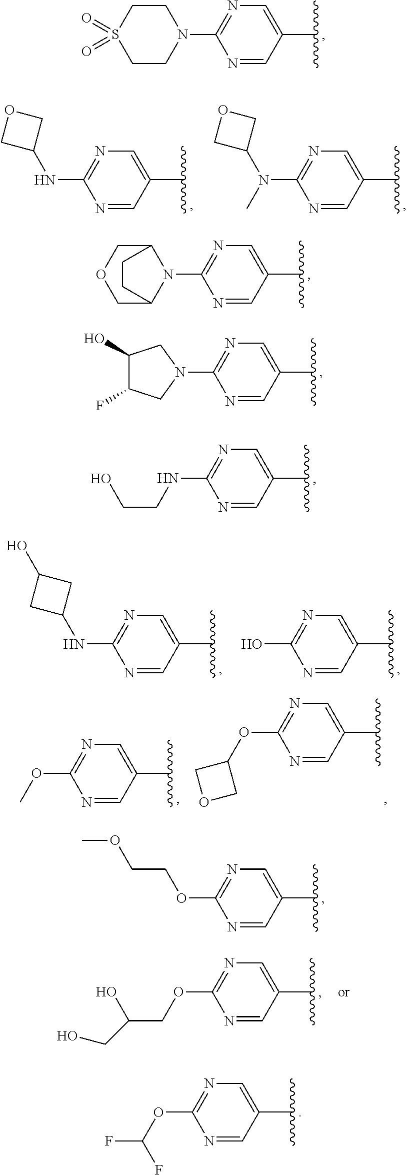

In some embodiments of a compound of Formula I or Ia-Ic,

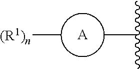

is

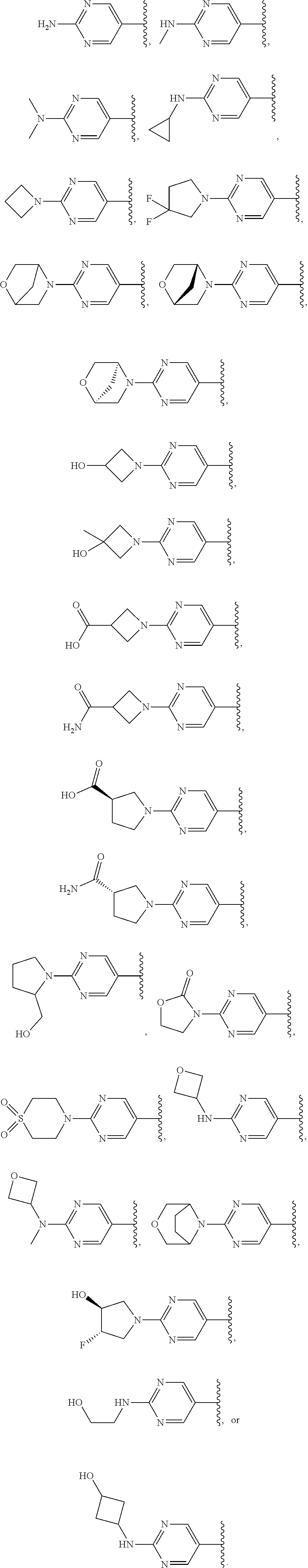

In some embodiments of a compound of Formula (I) or (Ia)-(Ic),

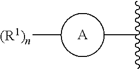

is

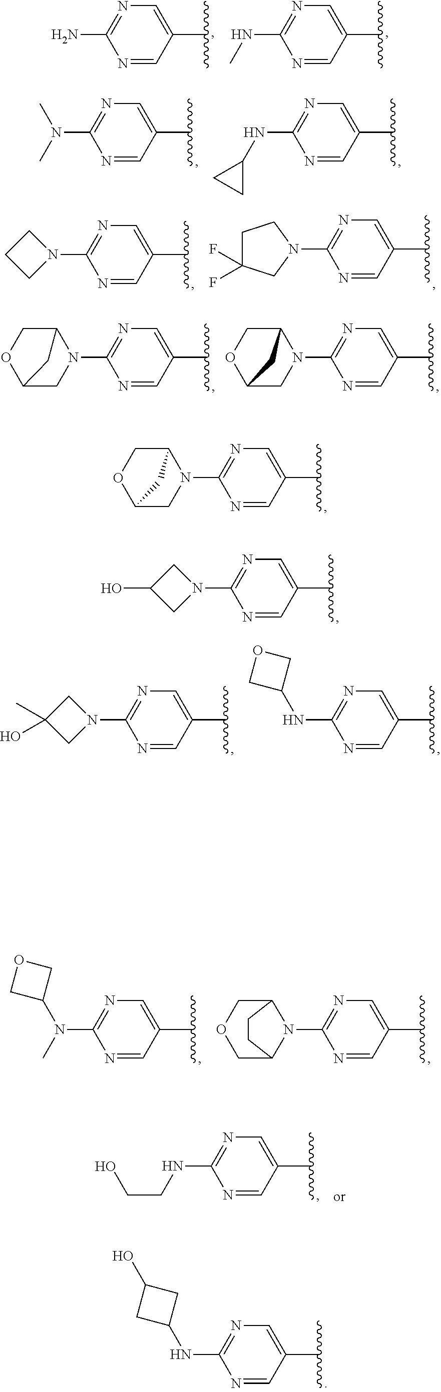

In some embodiments of a compound of Formula (I) or (Ia)-(Ic),

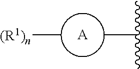

is

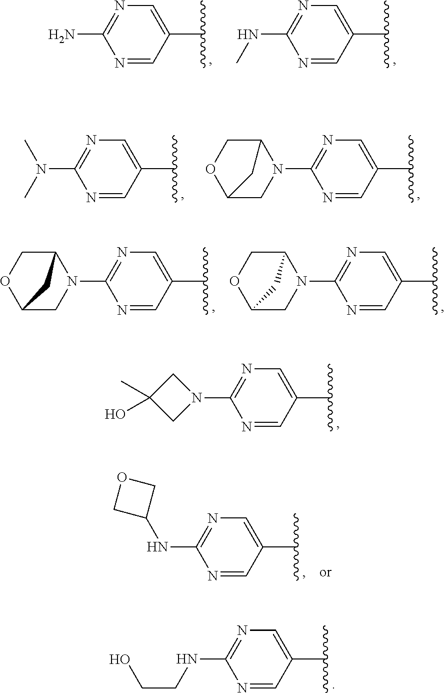

In some embodiments of a compound of Formula (I) or (Ia)-(Ic),

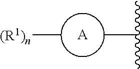

is

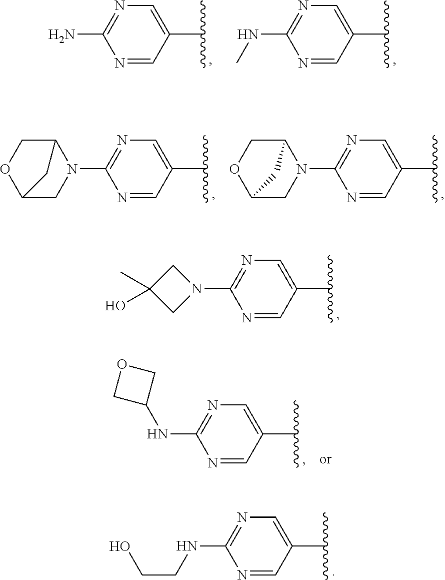

In some embodiments of a compound of Formula (I) or (Ia)-(Ic),

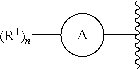

is

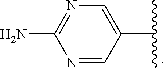 [SMILES: *c1cnc(N)nc1]

In some embodiments of a compound of Formula (I) or (Ia)-(Ic),

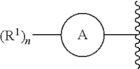

is

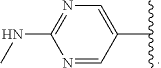 [SMILES: *c1cnc(NC)nc1]

In some embodiments of a compound of Formula (I) or (Ia)-(Ic)

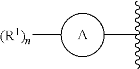

is

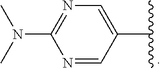 [SMILES: *c1cnc(N(C)C)nc1]

In some embodiments of a compound of Formula (I) or (Ia)-(Ic),

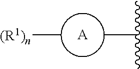

is

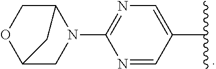 [SMILES: *c1cnc(N2CC3CC2CO3)nc1]

In some embodiments of a compound of Formula (I) or (Ia)-(Ic),

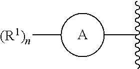

is

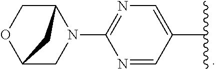 [SMILES: *c1cnc(N2C[C@H]3C[C@@H]2CO3)nc1]

In some embodiments of a compound of Formula (I) or (Ia)-(Ic),

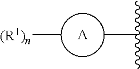

is

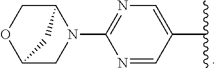 [SMILES: *c1cnc(N2C[C@@H]3C[C@H]2CO3)nc1]

In some embodiments of a compound of Formula (I) or (Ia)-(Ic),

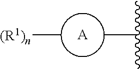

is

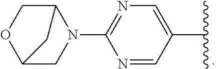 [SMILES: *c1cnc(N2CC3CC2CO3)nc1]

In some embodiments of a compound of Formula (I) or (Ia)-(Ic),

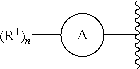

is

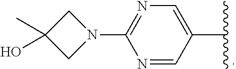 [SMILES: *c1cnc(N2CC(C)(O)C2)nc1]

In some embodiments of a compound of Formula (I) or (Ia)-(Ic),

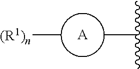

is

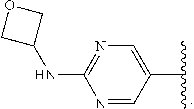 [SMILES: *c1cnc(NC2COC2)nc1]

In some embodiments of a compound of Formula (I) or (Ia)-(Ic),

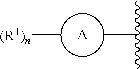

is

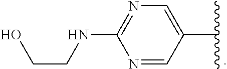 [SMILES: *c1cnc(NCCO)nc1]

In some embodiments of a compound of Formula (I) or (Ia)-(Ic),

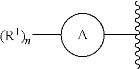

is

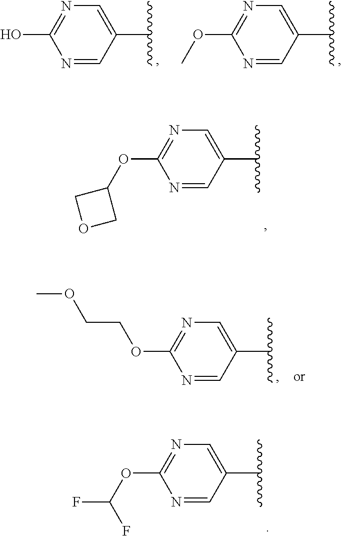

In some embodiments of a compound of Formula (I) or (Ia)-(Ic),

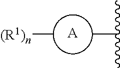

is

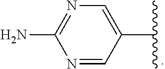 [SMILES: *c1cnc(N)nc1]

In some embodiments of a compound of Formula (I) or (Ia)-(Ic),

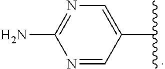 [SMILES: *c1cnc(N)nc1]

In some embodiments of a compound of Formula (I) or (Ia)-(Ic),

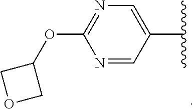 [SMILES: *c1cnc(OC2COC2)nc1]

In some embodiments of a compound of Formula (I) or (Ia)-(Ic),

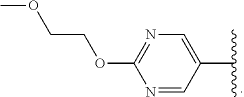 [SMILES: *c1cnc(OCCOC)nc1]

In some embodiments of a compound of Formula (I) or (Ia)-(Ic),

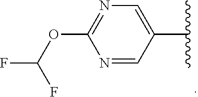 [SMILES: *c1cnc(OC(F)F)nc1]

In some embodiments of a compound of Formula (I) or (Ia)-(Ic),

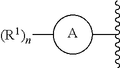

is

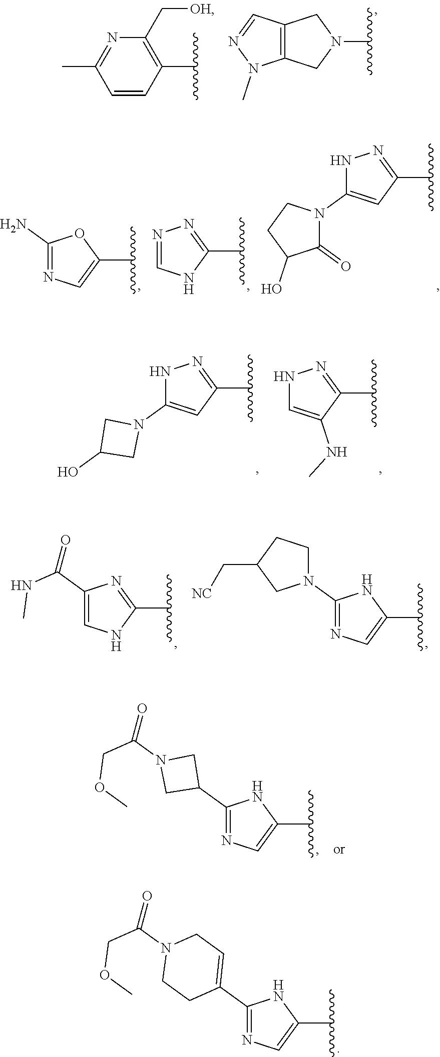

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound disclosed herein, each R^{a} is independently C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{a} is independently C₁-C₆alkyl, C₁-C₆haloalkyl, or cycloalkyl, heterocycloalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound disclosed herein, each R^{b} is independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{b} is independently hydrogen, C₁-C₆alkyl, C₁-C₆haloalkyl, or cycloalkyl, heterocycloalkyl. In some embodiments of a compound disclosed herein, each R^{b} is independently hydrogen, C₁-C₆alkyl or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₅alkylene(heteroaryl); wherein each alkyl, cycloalkyl, heterocycloalkyl, aryl, and heteroaryl is independently optionally substituted with one or more R. In some embodiments of a compound disclosed herein, each R^{c} and R^{d} are independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{c} and R^{d} are independently hydrogen, C₁-C₆alkyl, C₁-C₆haloalkyl, or cycloalkyl, heterocycloalkyl. In some embodiments of a compound disclosed herein, each R^{c} and R^{d} are independently hydrogen, C₁-C₆alkyl or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl; or two R on the same atom form an oxo. In some embodiments of a compound disclosed herein, each R is independently halogen, —CN, —OH, —NH₂, —NHCH₃, —N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl; or two R on the same atom form an oxo. In some embodiments of a compound disclosed herein, each R is independently halogen, —CN, —OH, C₁-C₆alkyl, C₁-C₆alkoxy, or C₁-C₆haloalkyl; or two R on the same atom form an oxo. In some embodiments of a compound disclosed herein, each R is independently halogen, —CN, —OH, or C₁-C₆alkyl; or two R on the same atom form an oxo. In some embodiments of a compound disclosed herein, each R is independently halogen, —OH, or C₁-C₆alkyl. In some embodiments of a compound disclosed herein, each R is independently halogen or C₁-C₆alkyl. In some embodiments of a compound disclosed herein, each R is independently halogen.

In some embodiments of a compound disclosed herein, one or more of R, R¹, R^{1a}, R², R³, R⁴, R⁵, R⁶, R^{X}, R^{Y}, R^{Z}, R^{W}, R^{a}, R^{b}, R^{c}, and R^{d} groups comprise deuterium at a percentage higher than the natural abundance of deuterium.

In some embodiments of a compound disclosed herein, one or more ¹H are replaced with one or more deuteriums in one or more of the following groups R, R¹, R^{1a}, R², R³, R⁴, R⁵, R⁶, R^{X}, R^{Y}, R^{Z}, R^{W}, R^{a}, R^{b}, R^{c}, and R^{d}.

In some embodiments of a compound disclosed herein, the abundance of deuterium in each of R, R¹, R^{1a}, R², R³, R⁴, R⁵, R⁶, R^{X}, R^{Y}, R^{Z}, R^{W}, R^{a}, R^{b}, R^{c}, and R^{d} is independently at least 1%, at least 10%, at least 20%, at least 30%, at least 40%, at least 50%, at least 60%, at least 70%, at least 80%, at least 90%, or 100% by molar.

In some embodiments of a compound disclosed herein, one or more hydrogens of Ring A are replaced with one or more deuteriums.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt thereof, is one of the compounds in Table 1.

TABLE 1

| Example | Structure |
|---|---|
| 1 | 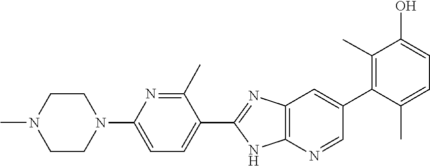 [SMILES: Cc1ccc(O)c(C)c1-c1cnc2[nH]c(-c3ccc(N4CCN(C)CC4)nc3C)nc2c1] |
| 2 | 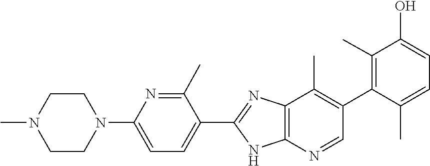 [SMILES: Cc1ccc(O)c(C)c1-c1cnc2[nH]c(-c3ccc(N4CCN(C)CC4)nc3C)nc2c1C] |
| 3 | 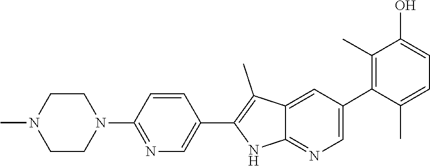 [SMILES: Cc1ccc(O)c(C)c1-c1cnc2[nH]c(-c3ccc(N4CCN(C)CC4)nc3)c(C)c2c1] |
| 4 | 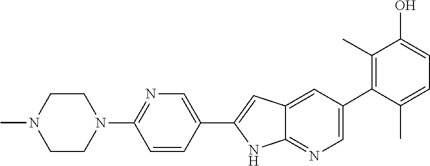 [SMILES: Cc1ccc(O)c(C)c1-c1cnc2[nH]c(-c3ccc(N4CCN(C)CC4)nc3)cc2c1] |
| 5 | 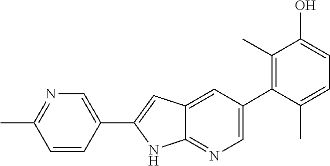 [SMILES: Cc1ccc(-c2cc3cc(-c4c(C)ccc(O)c4C)cnc3[nH]2)cn1] |
| 6 | 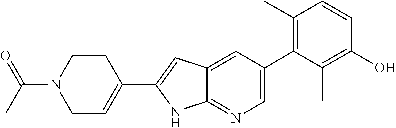 [SMILES: CC(=O)N1CC=C(c2cc3cc(-c4c(C)ccc(O)c4C)cnc3[nH]2)CC1] |
| 7 | 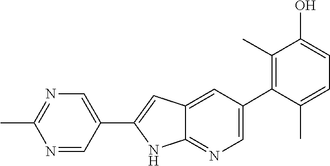 [SMILES: Cc1ncc(-c2cc3cc(-c4c(C)ccc(O)c4C)cnc3[nH]2)cn1] |
| 8 | 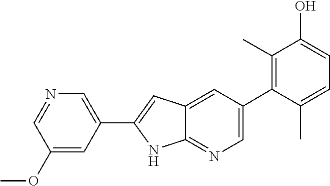 [SMILES: COc1cncc(-c2cc3cc(-c4c(C)ccc(O)c4C)cnc3[nH]2)c1] |
| 9 | 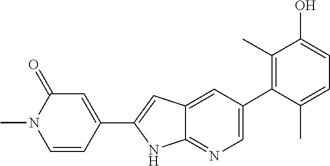 [SMILES: Cc1ccc(O)c(C)c1-c1cnc2[nH]c(-c3ccn(C)c(=O)c3)cc2c1] |
| 10 | 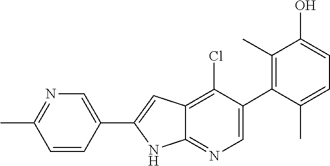 [SMILES: Cc1ccc(-c2cc3c(Cl)c(-c4c(C)ccc(O)c4C)cnc3[nH]2)cn1] |
| 11 | 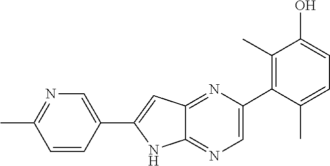 [SMILES: Cc1ccc(-c2cc3nc(-c4c(C)ccc(O)c4C)cnc3[nH]2)cn1] |
| 12 | 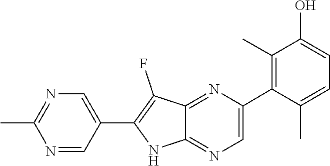 [SMILES: Cc1ncc(-c2[nH]c3ncc(-c4c(C)ccc(O)c4C)nc3c2F)cn1] |
| 13 | 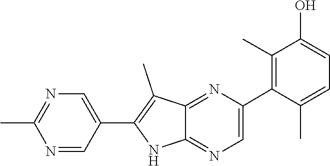 [SMILES: Cc1ncc(-c2[nH]c3ncc(-c4c(C)ccc(O)c4C)nc3c2C)cn1] |
| 14 | 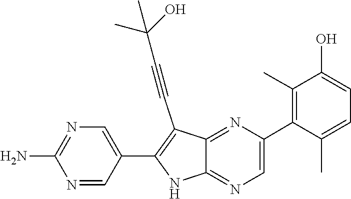 [SMILES: Cc1ccc(O)c(C)c1-c1cnc2[nH]c(-c3cnc(N)nc3)c(C#CC(C)(C)O)c2n1] |
| 15 | 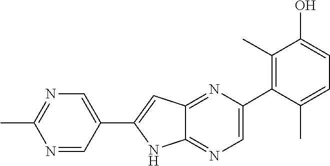 [SMILES: Cc1ncc(-c2cc3nc(-c4c(C)ccc(O)c4C)cnc3[nH]2)cn1] |
| 16 | 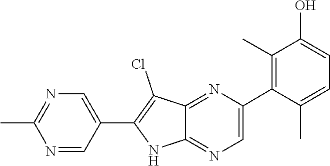 [SMILES: Cc1ncc(-c2[nH]c3ncc(-c4c(C)ccc(O)c4C)nc3c2Cl)cn1] |
| 17 | 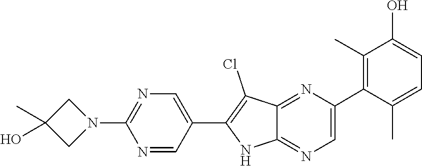 [SMILES: Cc1ccc(O)c(C)c1-c1cnc2[nH]c(-c3cnc(N4CC(C)(O)C4)nc3)c(Cl)c2n1] |
| 18 | 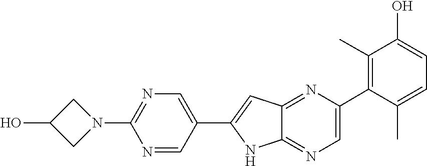 [SMILES: Cc1ccc(O)c(C)c1-c1cnc2[nH]c(-c3cnc(N4CC(O)C4)nc3)cc2n1] |
| 19 | 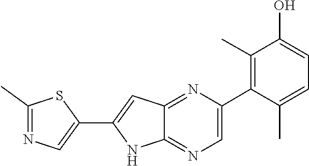 [SMILES: Cc1ncc(-c2cc3nc(-c4c(C)ccc(O)c4C)cnc3[nH]2)s1] |
| 20 | 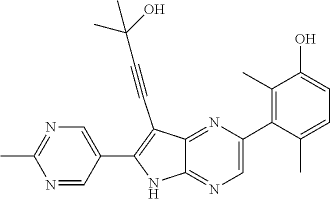 [SMILES: Cc1ncc(-c2[nH]c3ncc(-c4c(C)ccc(O)c4C)nc3c2C#CC(C)(C)O)cn1] |
| 21 | 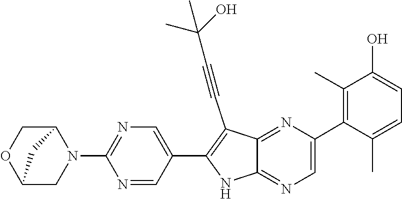 [SMILES: Cc1ccc(O)c(C)c1-c1cnc2[nH]c(-c3cnc(N4C[C@@H]5C[C@H]4CO5)nc3)c(C#CC(C)(C)O)c2n1] |
| 22 | 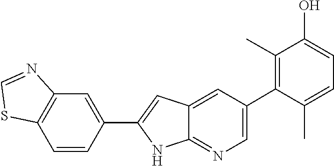 [SMILES: Cc1ccc(O)c(C)c1-c1cnc2[nH]c(-c3ccc4scnc4c3)cc2c1] |
| 23 | 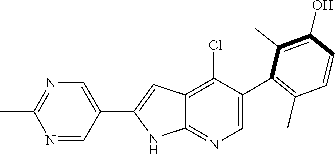 |
| 24 | 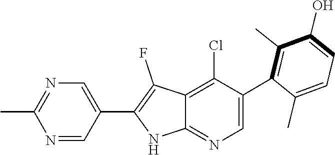 |
| 25 | 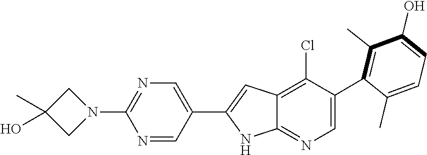 |
| 26 | 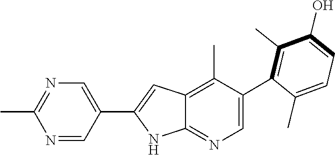 |
| 27 | 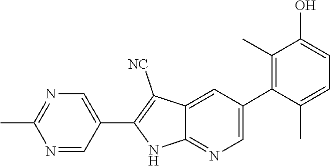 [SMILES: Cc1ncc(-c2[nH]c3ncc(-c4c(C)ccc(O)c4C)cc3c2C#N)cn1] |
| 29 | 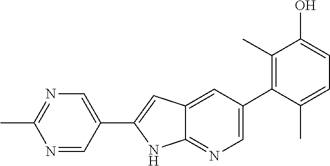 [SMILES: Cc1ncc(-c2cc3cc(-c4c(C)ccc(O)c4C)cnc3[nH]2)cn1] |
| 30 | 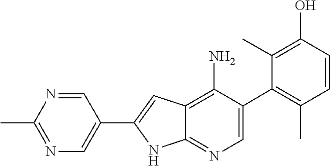 [SMILES: Cc1ncc(-c2cc3c(N)c(-c4c(C)ccc(O)c4C)cnc3[nH]2)cn1] |
| 31 | 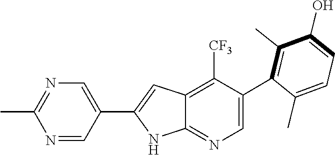 |
| 32 | 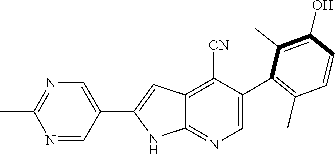 |
| 33 | 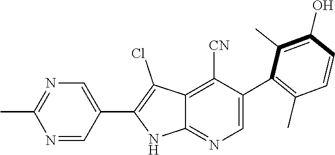 |
| 34 | 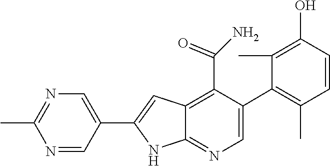 [SMILES: Cc1ncc(-c2cc3c(C(N)=O)c(-c4c(C)ccc(O)c4C)cnc3[nH]2)cn1] |
| 35 | 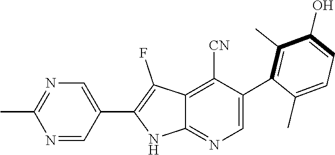 |
| 36 | 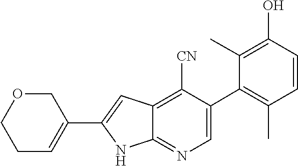 [SMILES: Cc1ccc(O)c(C)c1-c1cnc2[nH]c(C3=CCCOC3)cc2c1C#N] |
| 37 | 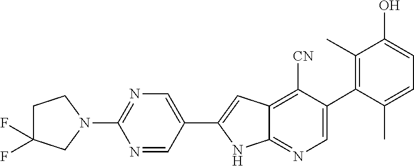 [SMILES: Cc1ccc(O)c(C)c1-c1cnc2[nH]c(-c3cnc(N4CCC(F)(F)C4)nc3)cc2c1C#N] |
| 38 | 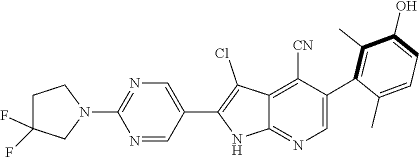 |
| 39 | 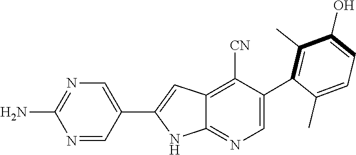 |
| 40 | 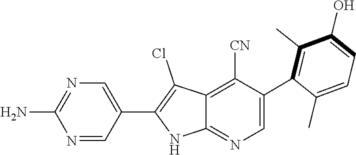 |
| 41 | 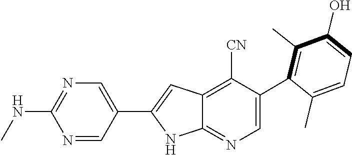 |
| 42 | 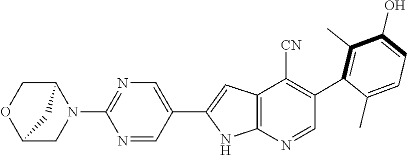 |
| 43 | 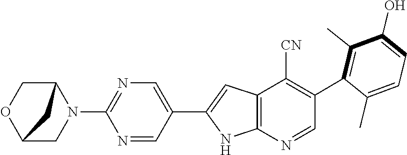 |
| 44 | 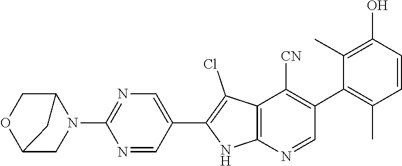 [SMILES: Cc1ccc(O)c(C)c1-c1cnc2[nH]c(-c3cnc(N4CC5CC4CO5)nc3)c(Cl)c2c1C#N] |
| 45 | 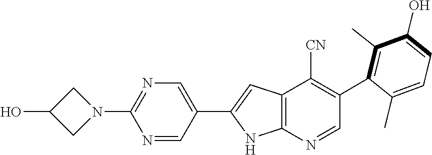 |
| 46 | 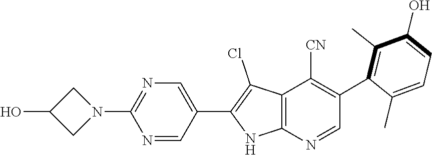 |
| 47 | 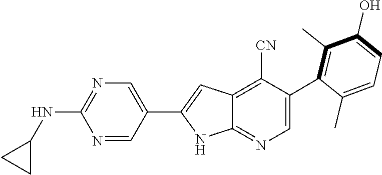 |
| 48 | 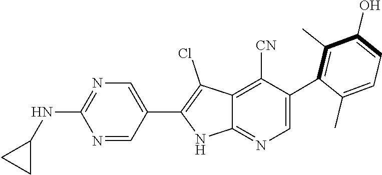 |
| 49 | 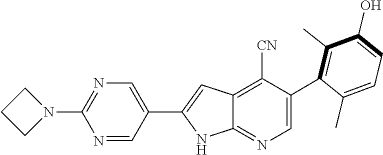 |
| 50 | 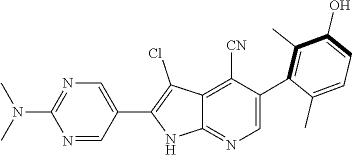 |
| 51 | 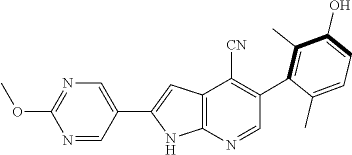 |
| 52 | 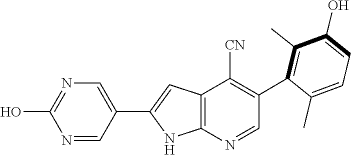 |
| 53 | 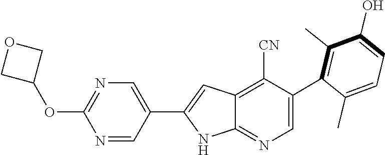 |
| 54 | 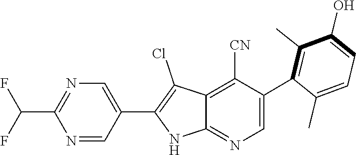 |
| 55 | 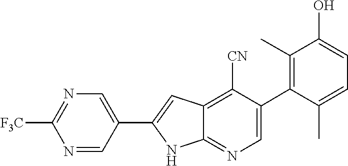 [SMILES: Cc1ccc(O)c(C)c1-c1cnc2[nH]c(-c3cnc(C(F)(F)F)nc3)cc2c1C#N] |
| 56 | 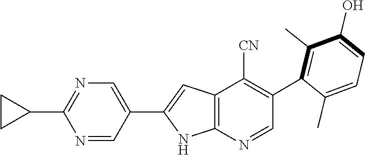 |
| 57 | 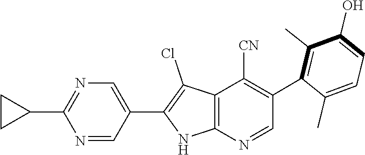 |
| 58 | 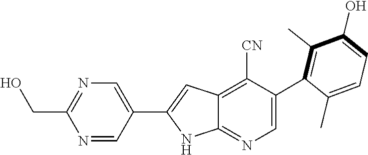 |
| 59 | 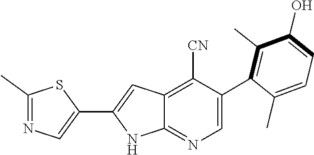 |
| 60 | 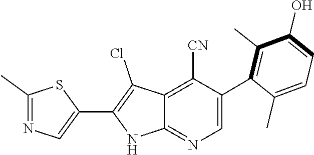 |
| 61 | 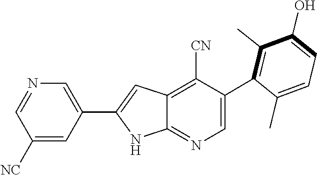 |
| 62 | 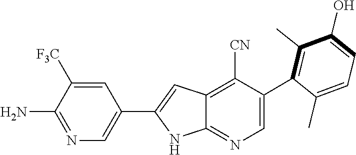 |
| 63 | 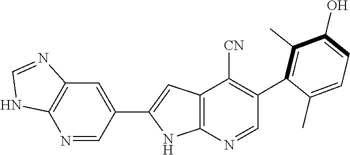 |
| 64 | 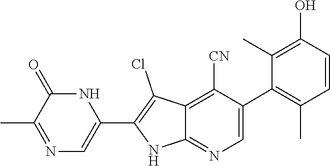 [SMILES: Cc1ccc(O)c(C)c1-c1cnc2[nH]c(-c3cnc(C)c(=O)[nH]3)c(Cl)c2c1C#N] |
| 65 | 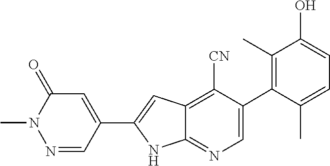 [SMILES: Cc1ccc(O)c(C)c1-c1cnc2[nH]c(-c3cnn(C)c(=O)c3)cc2c1C#N] |
| 66 | 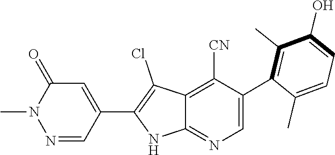 |
| 67 | 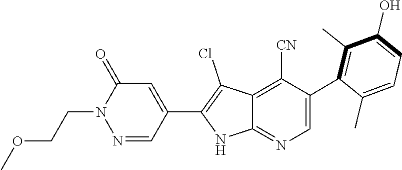 |
| 68 | 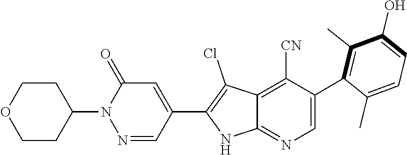 |
| 69 | 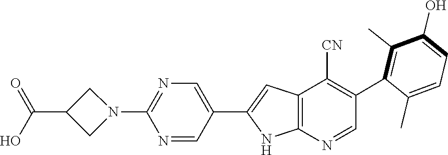 |
| 70 | 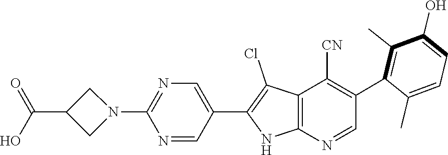 |
| 71 | 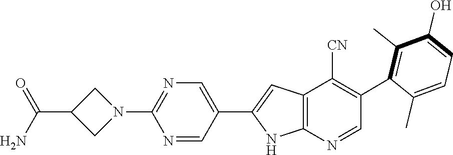 |
| 72 | 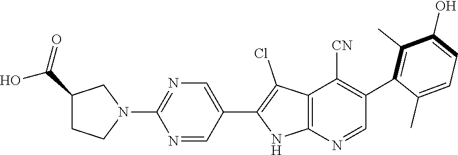 |
| 73 | 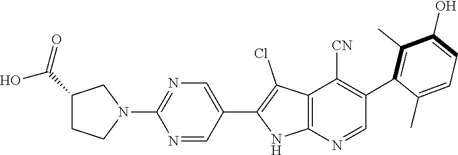 |
| 74 | 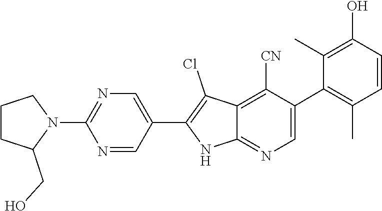 [SMILES: Cc1ccc(O)c(C)c1-c1cnc2[nH]c(-c3cnc(N4CCCC4CO)nc3)c(Cl)c2c1C#N] |
| 75 | 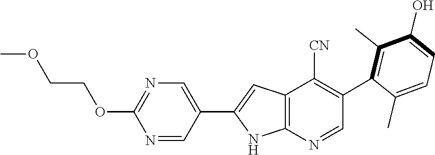 |
| 76 | 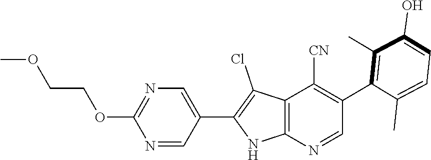 |
| 77 | 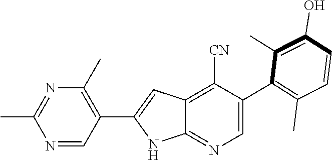 |
| 78 | 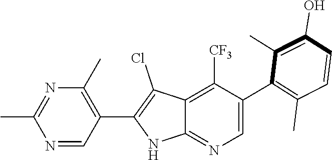 |
| 79 | 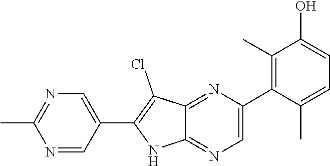 [SMILES: Cc1ncc(-c2[nH]c3ncc(-c4c(C)ccc(O)c4C)nc3c2Cl)cn1] |
| 80 | 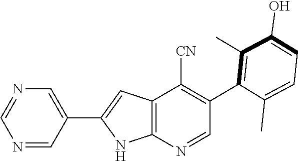 |
| 81 | 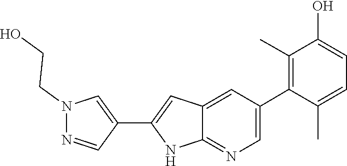 [SMILES: Cc1ccc(O)c(C)c1-c1cnc2[nH]c(-c3cnn(CCO)c3)cc2c1] |
| 82 | 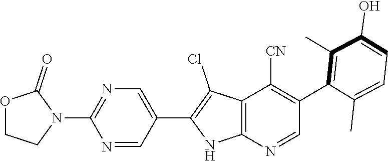 |
| 83 | 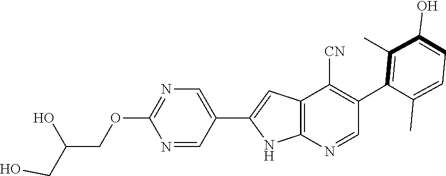 |
| 84 | 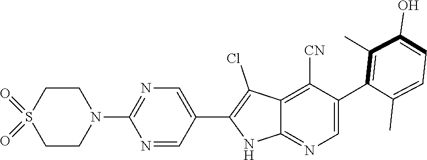 |
| 85 | 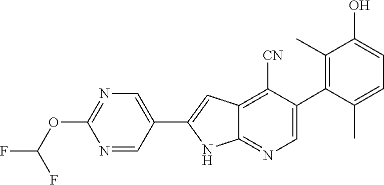 [SMILES: Cc1ccc(O)c(C)c1-c1cnc2[nH]c(-c3cnc(OC(F)F)nc3)cc2c1C#N] |
| 86 | 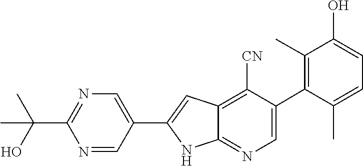 [SMILES: Cc1ccc(O)c(C)c1-c1cnc2[nH]c(-c3cnc(C(C)(C)O)nc3)cc2c1C#N] |
| 87 | 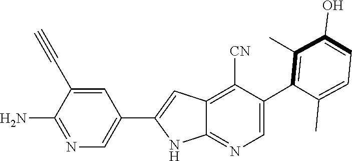 |
| 88 | 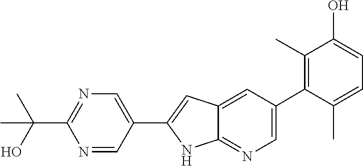 [SMILES: Cc1ccc(O)c(C)c1-c1cnc2[nH]c(-c3cnc(C(C)(C)O)nc3)cc2c1] |
| 89 | 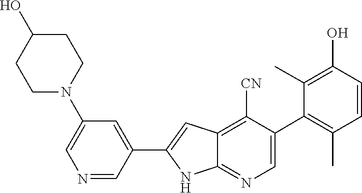 [SMILES: Cc1ccc(O)c(C)c1-c1cnc2[nH]c(-c3cncc(N4CCC(O)CC4)c3)cc2c1C#N] |
| 90 | 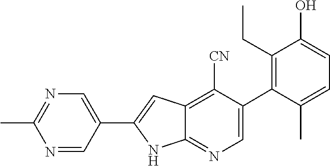 [SMILES: CCc1c(O)ccc(C)c1-c1cnc2[nH]c(-c3cnc(C)nc3)cc2c1C#N] |
| 91 | 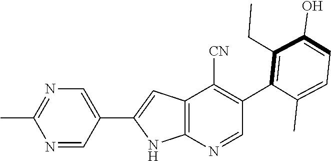 |
| 92 | 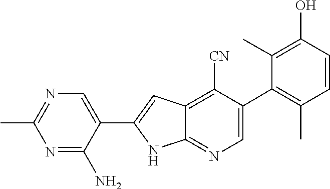 [SMILES: Cc1ncc(-c2cc3c(C#N)c(-c4c(C)ccc(O)c4C)cnc3[nH]2)c(N)n1] |
| 93 | 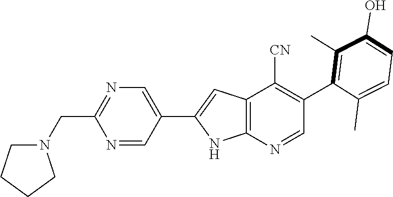 |
| 94 | 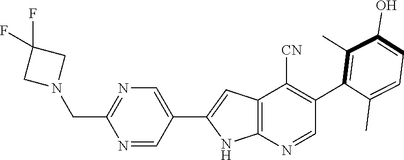 |
| 95 | 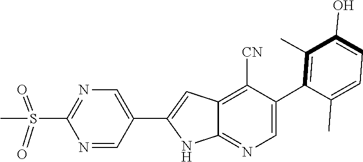 |
| 96 | 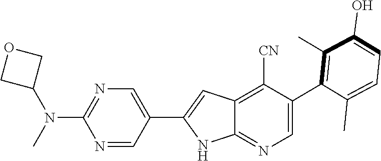 |
| 97 | 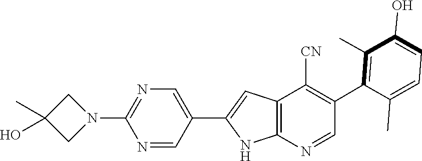 |
| 98 | 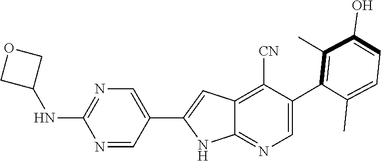 |
| 99 | 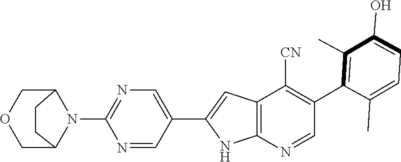 |
| 100 | 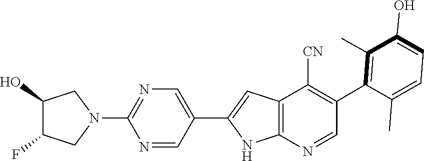 |
| 101 | 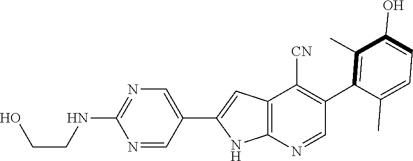 |
| 102 | 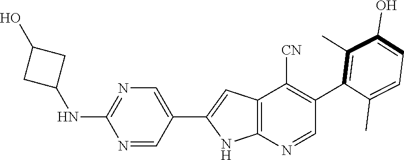 |
| | (unknown cis or trans isomer) |
| 103 | 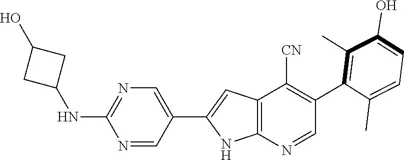 |
| | (unknown cis or trans isomer) |
| 104 | 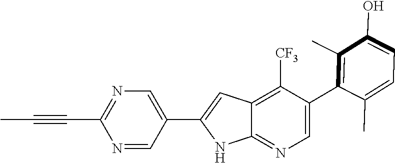 |
| 105 | 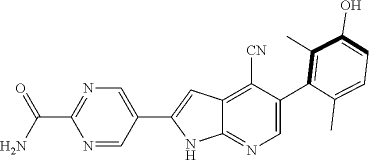 |
| 106 | 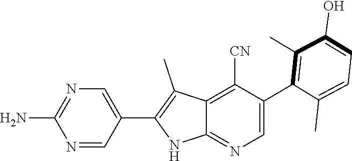 |
| 107 | 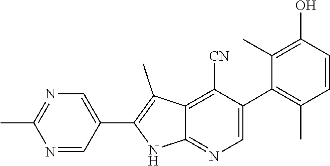 [SMILES: Cc1ncc(-c2[nH]c3ncc(-c4c(C)ccc(O)c4C)c(C#N)c3c2C)cn1] |
| 108 | 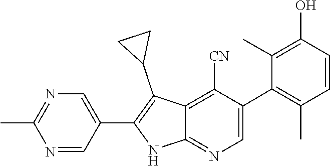 [SMILES: Cc1ncc(-c2[nH]c3ncc(-c4c(C)ccc(O)c4C)c(C#N)c3c2C2CC2)cn1] |
| 109 | 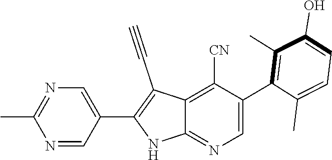 |
| 110 | 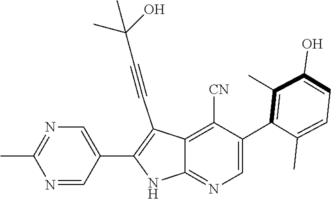 |
| 111 | 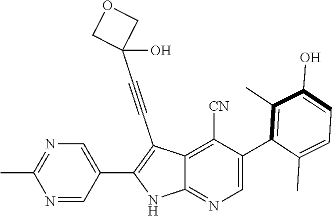 |
| 112 | 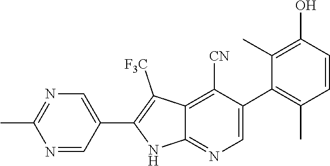 [SMILES: Cc1ncc(-c2[nH]c3ncc(-c4c(C)ccc(O)c4C)c(C#N)c3c2C(F)(F)F)cn1] |
| 113 | 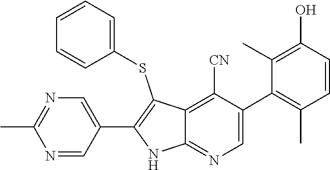 [SMILES: Cc1ncc(-c2[nH]c3ncc(-c4c(C)ccc(O)c4C)c(C#N)c3c2Sc2ccccc2)cn1] |
| 114 | 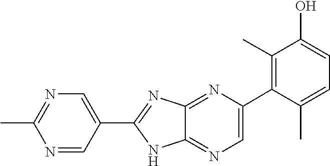 [SMILES: Cc1ncc(-c2nc3nc(-c4c(C)ccc(O)c4C)cnc3[nH]2)cn1] |
| 115 | 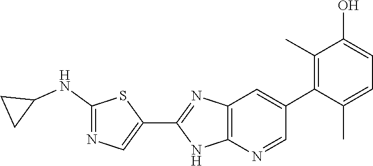 [SMILES: Cc1ccc(O)c(C)c1-c1cnc2[nH]c(-c3cnc(NC4CC4)s3)nc2c1] |
| 116 | 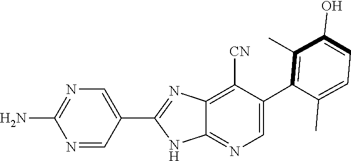 |
| 117 | 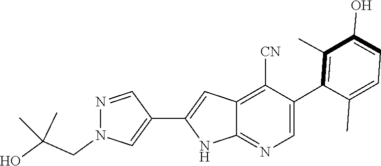 |
| 118 | 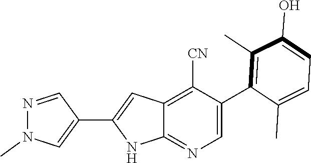 |
| 119 | 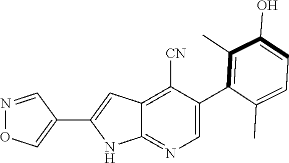 |
| 120 | 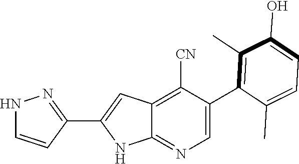 |
| 121 | 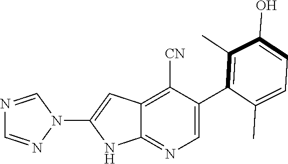 |
| 122 | 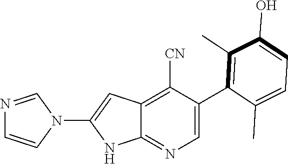 |
| 123 | 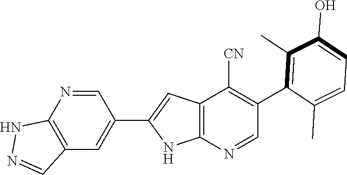 |
| 124 | 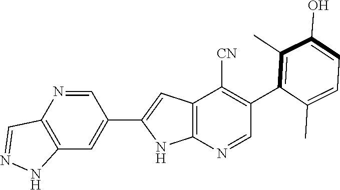 |
| 125 | 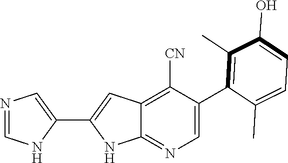 |
| 126 | 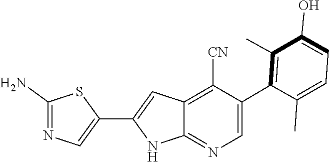 |
| 127 | 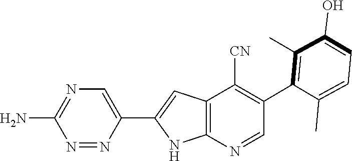 |
| 128 | 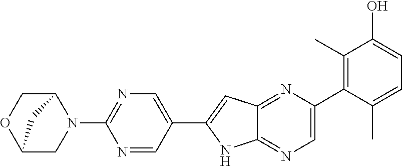 [SMILES: Cc1ccc(O)c(C)c1-c1cnc2[nH]c(-c3cnc(N4C[C@@H]5C[C@H]4CO5)nc3)cc2n1] |
| 129 | 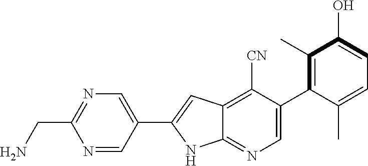 |
| 130 | 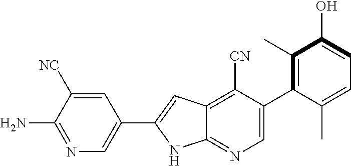 |
| 131 | 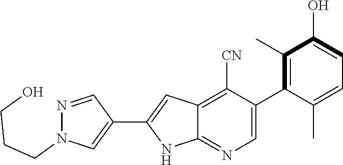 |
| 132 | 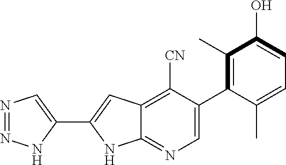 |
| 133 | 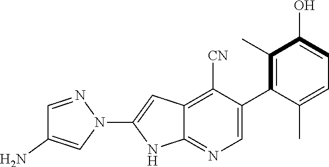 |
| 134 | 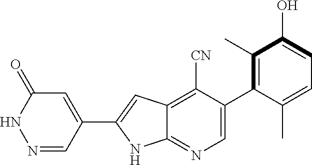 |
| 135 | 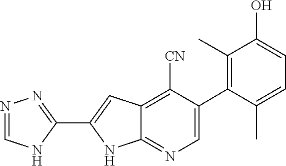 [SMILES: Cc1ccc(O)c(C)c1-c1cnc2[nH]c(-c3nnc[nH]3)cc2c1C#N] |

In some embodiments the compound disclosed herein, or a pharmaceutically acceptable salt thereof, is one of the compounds in Table 2.

TABLE 2
Structure

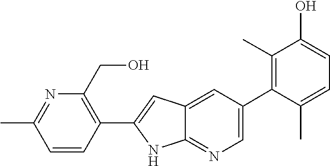 [SMILES: Cc1ccc(-c2cc3cc(-c4c(C)ccc(O)c4C)cnc3[nH]2)c(CO)n1]

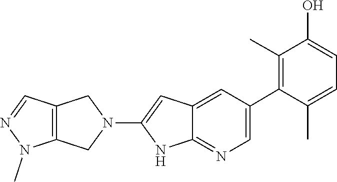 [SMILES: Cc1ccc(O)c(C)c1-c1cnc2[nH]c(N3Cc4cnn(C)c4C3)cc2c1]

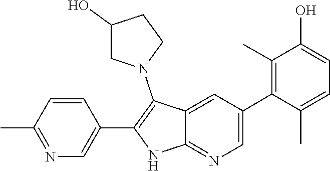 [SMILES: Cc1ccc(-c2[nH]c3ncc(-c4c(C)ccc(O)c4C)cc3c2N2CCC(O)C2)cn1]

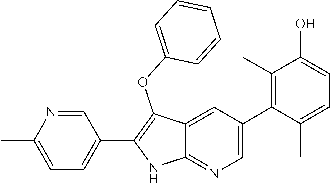 [SMILES: Cc1ccc(-c2[nH]c3ncc(-c4c(C)ccc(O)c4C)cc3c2Oc2ccccc2)cn1]

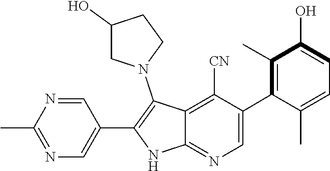

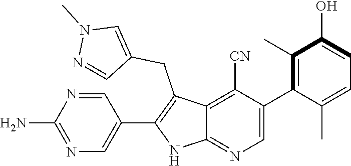

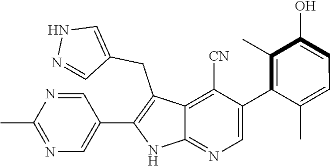

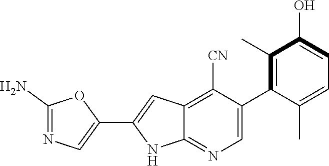

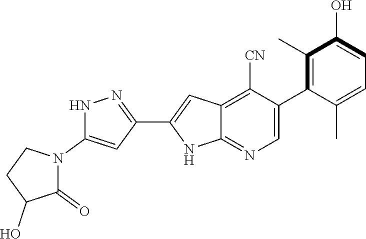

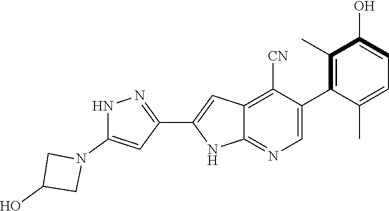

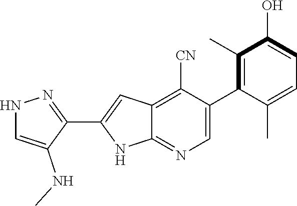

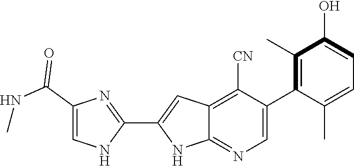

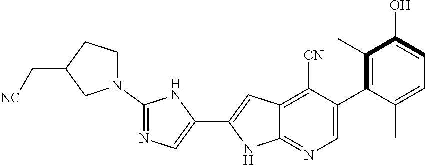

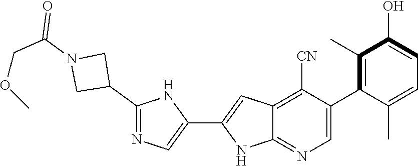

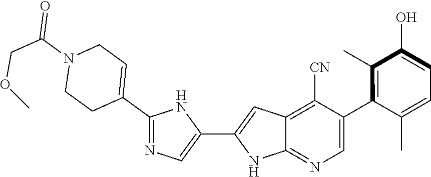

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

In some embodiments, the compounds described herein contain bonds with hindered rotation such that two separate rotamers or atropisomers can be isolated. In some embodiments the atropisomers are

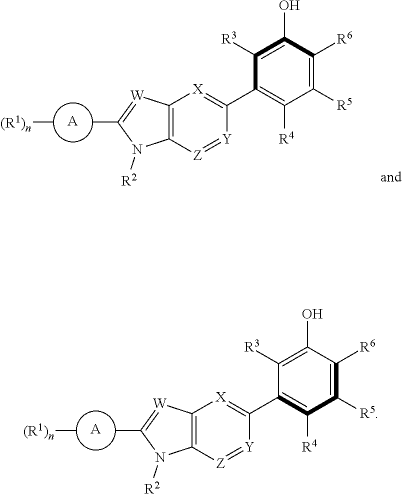

In some embodiments, these atropisomer are separated and are found to have different biological activity which may be advantageous. In some embodiments the atropisomer is

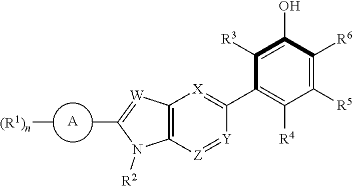

In some embodiments the atropisomer is

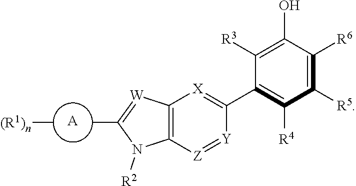

In some embodiments the compound disclosed herein is a racemic mixture:

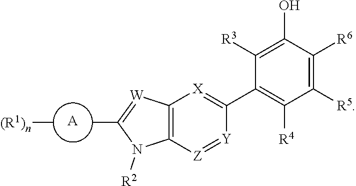

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H (D), ³H, ¹³C, ¹⁴C, ¹⁵N, ¹⁸O ¹⁷O, ³¹P, ³²P, ³⁵S, ⁸F, and ³⁶Cl, respectively. Compounds described herein, and the pharmaceutically acceptable salts thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% of a total number of hydrogen and deuterium. In some embodiments, one or more of the substituents disclosed herein comprise deuterium at a percentage higher than the natural abundance of deuterium. In some embodiments, one or more hydrogens are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein are methods of treating a disease modulated at least in part by PKMYT1 in a subject in need thereof, comprising administering to the subject a therapeutically affective amount of a compound, or a pharmaceutically acceptable salt thereof, disclosed herein.

Disclosed herein is a method of treating cancer in a subject in need thereof, the method comprising administering to the subject a therapeutically affective amount of a compound, or a pharmaceutically acceptable salt thereof, disclosed herein.

In some embodiments, the cancer depends on the activity of PKMYT1.

In some embodiments, the cancer overexpresses CCNE1. In some embodiments cancers which have a high incidence of CCNE1 overexpression include e.g., breast cancer, endometrial cancer, esophageal cancer, lung cancer, ovarian cancer, stomach cancer, and uterine cancer.

In some embodiments, the cancer has an inactivating mutation in the FBXW7 gene. In some embodiments cancers which have a deficiency in FBXW7 include, e.g., breast cancer, colorectal cancer, esophageal cancer, lung cancer, and uterine cancer.

In some embodiments, the cancer is a solid tumor.

In some embodiments, the cancer is breast cancer, colorectal cancer, endometrial cancer, esophageal cancer, glioblastoma, hepatocellular carcinoma, lung cancer, neuroblastoma, ovarian cancer, prostate cancer, stomach cancer, or uterine cancer.

Disclosed herein is a method of modulating PKMYT1 in a subject, the method comprising administering to the subject a compound, or a pharmaceutically acceptable salt thereof, disclosed herein. Disclosed herein is a method of inhibiting PKMYT1 in a subject, the method comprising administering to the subject a compound, or a pharmaceutically acceptable salt thereof, disclosed herein. Further disclosed herein is a method of selectively inhibiting PKMYT1 in a subject (e.g., selective over WEE1), the method comprising administering to the subject a compound, or a pharmaceutically acceptable salt thereof, disclosed herein.

WEE1 widely exists in human tissues and plays an important role in all phases of the cell cycle. It can be beneficial to use compounds that selectively inhibit PKMYT1 over WEE1 in methods of treatment described herein. In some embodiments, compounds disclosed herein do not inhibit WEE1. In some embodiments, a compound disclosed herein has a WEE1 IC50 value of at least about 100 nM, at least about 500 nM, at least about 1000 nM, or at least about 10,000 nM as determined in a WEE1 ADP-Glo assay. For example, the WEE1 IC50 value can be determined according to Example B.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In some embodiments, doses employed for adult human treatment are typically in the range of 0.01 mg-5000 mg per day. In some embodiments, the daily dosages appropriate for the compound described herein, or a pharmaceutically acceptable salt thereof, are from about 0.01 to about 50 mg/kg per body weight. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination

Disclosed herein are methods of treating a disease or disorder associated with PKMYT1 using a compound disclosed herein, or a pharmaceutically acceptable salt thereof, in combination with an additional therapeutic agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

In some embodiments, the additional therapeutic agent is an anti-cancer agent.

### EXAMPLES

### Example 1: Preparation of 2,4-dimethyl-3-(2-(2-methyl-6-(4-methylpiperazin-1-yl)pyridin-3-yl)-3H-imidazo[4,5-b]pyridin-6-yl)phenol

The title compound was prepared according to the following synthetic procedures.

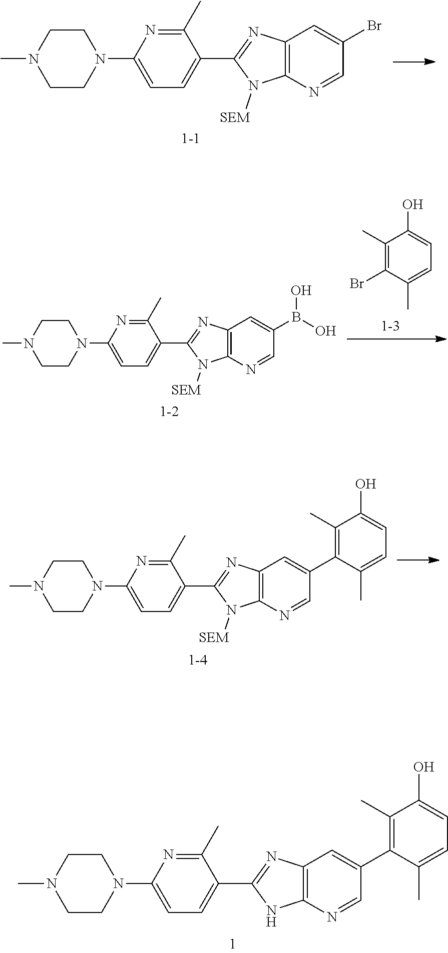

### Step 1: Preparation of Compound 1-2

To a solution of compound 1-1 (250 mg, 0.48 mmol) in dioxane (2 mL) were added Pin₂B₂ (245 mg, 0.97 mmol), KOAc (142 mg, 1.45 mmol) and Pd(dppf)Cl₂ (73 mg, 0.1 mmol). The mixture was stirred at 90° C. under N₂ for 6 h. The reaction mixture was cooled to room temperature, diluted with water (10 mL), extracted with EA (20 mL×3), washed with brine (80 mL), dried over Na₂SO₄, filtered, and concentrated to dryness. The crude compound 1-2 was used in next step. LCMS: 483.4 [M+H]⁺.

### Step 2: Preparation of Compound 1-4

To a solution of compound 1-2 (60 mg, 0.12 mmol) in dioxane (2 mL) and H₂O (0.2 mL) were added compound 1-3 (33 mg, 0.18 mmol), K₂CO₃ (49 mg, 0.36 mmol) and Pd(PPh₃)₄ (55 mg, 0.048 mmol). The mixture was stirred at 90° C. under N₂ for 3 h. The reaction mixture was filtered. The filtrate was concentrated to dryness. The residue was purified by prep-TLC to afford compound 1-4. LCMS: 559.3 [M+H]⁺.

### Step 3: Preparation of Example 1

To a solution of compound 1-4 (73 mg, 0.12 mmol) in dioxane (2 mL) was added TFA (1.0 mL). The mixture was stirred at 20° C. under N₂ for 1 h. The reaction mixture was concentrated, and purified by prep-HPLC to afford example 1. LCMS: 429.3 [M+H]⁺; ¹H NMR: (400 MHz, CD₃OD) δ 8.10 (s, 1H), 7.97 (d, J=8.6 Hz, 1H), 7.73 (s, 1H), 6.93 (dd, J=29.8, 8.5 Hz, 2H), 6.76 (d, J=8.3 Hz, 1H), 3.94 (s, 4H), 3.24 (s, 4H), 2.83 (d, J=14.8 Hz, 3H), 2.73 (s, 3H), 1.94 (s, 3H), 1.90 (s, 3H).

### Example 2: Preparation of 2,4-dimethyl-3-(7-methyl-2-(2-methyl-6-(4-methylpiperazin-1-yl)pyridin-3-yl)-3H-imidazo[4,5-b]pyridin-6-yl)phenol

The title compound was prepared according to the following synthetic procedures.

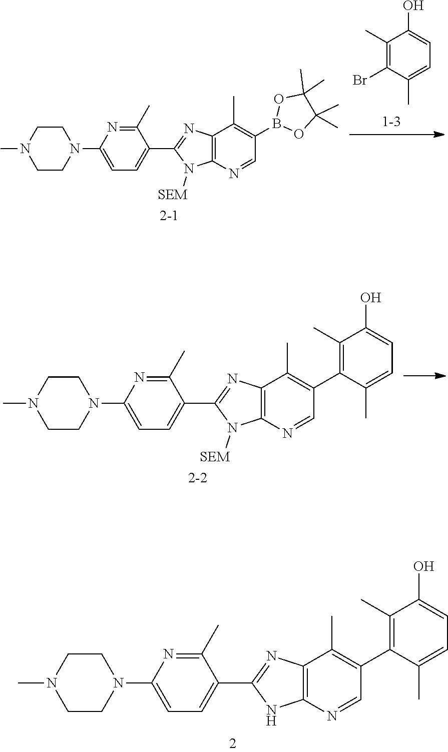

### Step 1: Preparation of Compound 2-2

To a solution of compound 2-1 (60 mg, 0.104 mmol) in dioxane (2 mL) were added compound 1-3 (20 mg, 0.104 mmol), pd(dtbpf)Cl₂ (67 mg, 0.104 mmol), Na₂CO₃ (10 mg, 0.104 mmol) and H₂O (0.2 mL). The reaction was stirred at 90° C. for 2 h under N₂. The reaction mixture was quenched with water (20 mL), and extracted with EA (3×20 mL). The organic layer was combined, washed with brine (50 mL), dried over Na₂SO₄, filtered and concentrated. The residue was purified by silica gel column chromatography to afford compound 2-2. LCMS: 573.3 [M+H]⁺.

### Step 2: Preparation of Example 2

To a stirred solution of compound 2-2 (50 mg, 0.087 mmol) in DCM (2 mL) were added TFA (0.5 mL). The reaction was stirred at 25° C. for 1 h under N₂. The reaction mixture was carefully added to ice water (20 mL), and adjusted to pH-8 with NaHCO₃. The mixture was extracted with DCM (30 mL×3). The organic layer was combined and concentrated to dryness. The residue was purified by prep-HPLC to give example 2. LCMS: 443.0 [M+H]⁺; ¹H NMR: (400 MHz, DMSO-d₆) δ 12.86 (d, J=188.9 Hz, 1H), 9.18 (s, 11H), 8.18-8.03 (m, 11H), 7.88 (d, J=28.6 Hz, 11H), 6.96 (d, J=8.1 Hz, 2H), 6.78 (d, J=8.2 Hz, 1H), 3.31-3.00 (m, 8H), 2.80 (s, 6H), 2.20 (s, 3H), 1.79 (s, 3H), 1.73 (s, 3H).

### Example 3: Preparation of 2,4-dimethyl-3-(3-methyl-2-(6-(4-methylpiperazin-1-yl)pyridin-3-yl)-1H-pyrrolo[2,3-b]pyridin-5-yl)phenol

The title compound was prepared according to the following synthetic procedures.

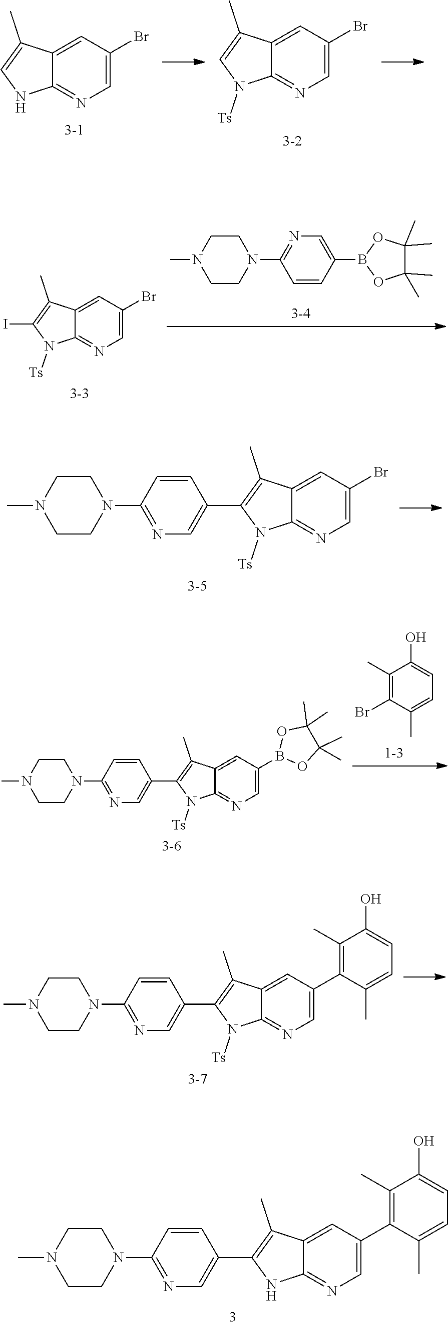

### Step 1: Preparation of Compound 3-2

To a solution of compound 3-1 (1.0 g, 4.738 mmol) in THF (20 mL) was added NaH (0.38 g 9.476 mmol) at 0° C. The mixture was stirred at 0° C. under N₂ for 0.5 h. Then TsCl (1.08 g, 1.780 mmol) was added. The mixture was stirred at 20° C. under N₂ for 1 h. The reaction was quenched by NH₄Cl (50 ml, sat. in water), extracted with EA (100 mL×3), washed with (100 mL), dried over Na₂SO₄, filtered, and concentrated under reduced pressure. The residue was purified by column chromatography on silica gel to afford compound 3-2 as a white solid. LCMS: 366.8 [M+H]⁺.

### Step 2: Preparation of Compound 3-3

To a solution of compound 3-2 (500 mg, 1.369 mmol) in THF (10 mL) was added LDA (1.03 mL) at −78° C. under N₂. The mixture was stirred at −78° C. under N₂ for 0.5 h. Then I₂ (451 mg, 1.780 mmol) was added. The mixture was stirred at −78° C. under N₂ for 1 h. The reaction was quenched by NH₄Cl (50 ml, sat. in water), which was extracted with EA (100 mL×3), washed with brine (100 mL), dried over Na₂SO₄, filtered, and concentrated under reduced pressure. The residue was purified by column chromatography on silica gel to afford compound 3-3 as a white solid. LCMS: 492.6 [M+H]⁺.

### Step 3: Preparation of Compound 3-5

To a solution of compound 3-3 (200 mg, 0.407 mmol) in dioxane (6 mL) and H₂O (1 mL) was added compound 3-4 (135 mg, 0.448 mmol), Pd(dppf)Cl₂ (30 mg, 0.041 mmol) and CsF (186 mg, 1.221 mmol). The mixture was stirred at 90° C. under N₂ for 1.5 h. The mixture was diluted with EA (100 mL), which was washed with brine (100 mL×3), dried over Na₂SO₄, filtered and concentrated. The residue was purified by column chromatography on silica gel to afford compound 3-5. LCMS: 542.2 [M+H]⁺.

### Step 4: Preparation of Compound 3-6

To a solution of compound 3-5 (77 mg, 0.143 mmol) in dioxane (2 mL) was added Pin₂B₂ (72 mg, 0.285 mmol), KOAc (42 mg, 0.428 mmol) and Pd(dppf)Cl₂ (11 mg, 0.014 mmol). The mixture was reacted at 90° C. under N₂ for 1.5 h, cooled to room temperature, diluted with water (10 mL), extracted with EA (20 mL×3), washed with brine (60 mL), dried over Na₂SO₄, filtered, and concentrated to dryness. The crude 3-6 was used directly in next step. LCMS: 588.3 [M+H]⁺.

### Step 5: Preparation of Compound 3-7

To a solution of compound 3-6 (54 mg, 0.009 mmol) in dioxane (2 mL) and H₂O (0.3 mL) was added compound 1-3 (43 mg, 0.214 mmol), K₂CO₃ (59 mg, 0.429 mmol) and Pd(dtbpf)Cl₂ (9.5 mg, 0.014 mmol). The mixture was stirred at 90° C. under N₂ for 1.5 h, cooled to room temperature, and filtered. The filtrate was concentrated and purified by column chromatography on silica gel to afford compound 3-7. LCMS: 582.3 [M+H]⁺.

### Step 6: Preparation of Example 3

To a solution of compound 3-7 (55 mg, 0.095 mmol) in EtOH (2 mL) was added NaOH (0.5 mL, 1 M in water). The mixture was stirred at 60° C. under N₂ for 5 h. The reaction mixture was concentrated to dryness. The residue was purified by prep-HPLC to afford example 3. LCMS: 428.0 [M+H]⁺. ¹H NMR: (400 MHz, DMSO-d₆) δ 11.69 (s, 1H), 9.12 (s, 1H), 8.49 (s, 1H), 7.88 (d, J=9.3 Hz, 2H), 7.62 (s, 1H), 7.00 (d, J=12.0 Hz, 1H), 6.93 (d, J=8.0 Hz, 1H), 6.74 (d, J=8.1 Hz, 1H), 3.61 (s, 4H), 2.48-2.45 (m, 3H), 2.36 (s, 3H), 2.30 (d, J=8.6 Hz, 4H), 1.88 (s, 3H), 1.81 (s, 3H).

### Example 4: Preparation of 2,4-dimethyl-3-(2-(6-(4-methylpiperazin-1-yl)pyridin-3-yl)-1H-pyrrolo[2,3-b]pyridin-5-yl)phenol

The title compound was prepared according to the following synthetic procedures.

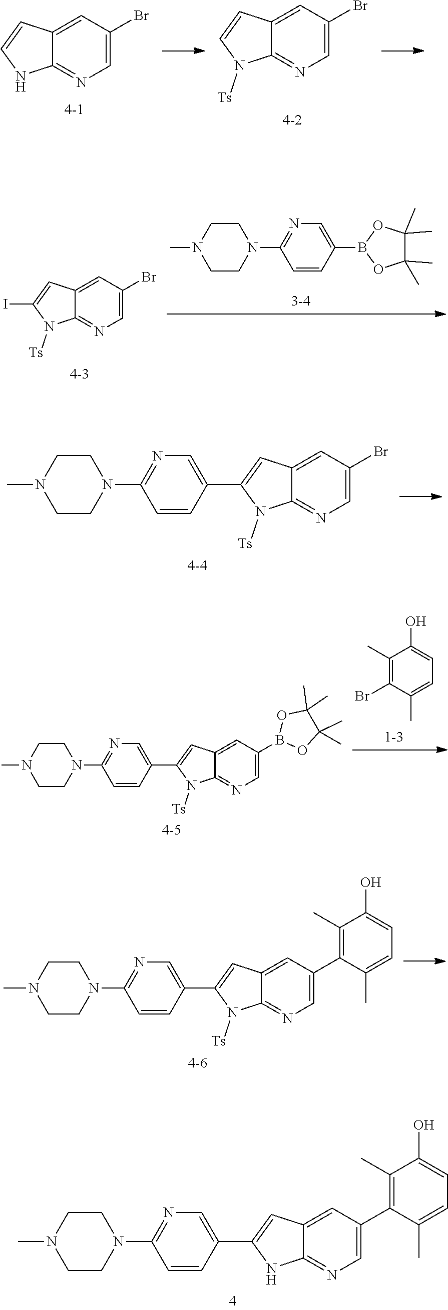

### Step 1: Preparation of Compound 4-2

To a solution of compound 4-1 (3 g, 15.23 mmol) in THF (60 mL) was added NaH (0.73 g, 30.45 mmol) at 0° C. The mixture was stirred at 0° C. under N₂ for 0.5 h. Then TsCl (3.48 g, 18.27 mmol) was added. The mixture was stirred at 20° C. under N₂ for 1 h. The reaction was quenched by NH₄Cl (50 mL, sat.), extracted with EA (100 mL×3), washed with brine (100 mL), dried over Na₂SO₄, filtered, and concentrated under reduced pressure. The residue was purified by column chromatography on silica gel to afford compound 4-2 as a white solid. LCMS: 352.7 [M+H]⁺.

### Step 2: Preparation of Compound 4-3

To a solution of compound 4-2 (1 g, 2.390 mmol) in THF (10 mL) was added LDA (0.46 g, 4.271 mmol) at −78° C. The mixture was stirred at −78° C. under N₂ for 0.5 h. Then I₂ (1.64 g, 3.701 mmol) was added. The mixture was stirred at −78° C. under N₂ for 1 h. The reaction was quenched by NH₄Cl (50 ml, sat.), extracted with EA (100 mL×3), washed with brine (100 mL), dried over Na₂SO₄, filtered, and concentrated under reduced pressure. The residue was purified by column chromatography on silica gel to compound 4-3 as a white solid. LCMS: 476.9 [M+H]⁺.

### Step 3: Preparation of Compound 4-4

To a solution of compound 4-3 (900 mg, 1.886 mmol) in dioxane (6 mL) and H₂O (1 mL) was added compound 3-4 (629 mg, 2.075 mmol), CsF (860 mg, 5.659 mmol) and Pd(dppf)Cl₂ (69 mg, 0.094 mmol). The mixture was stirred at 90° C. under N₂ for 1.5 h. The mixture was filtered. The filtrate was concentrated and purified by column chromatography on silica gel to afford compound 4-4. LCMS: 528.1 [M+H]⁺.

### Step 4: Preparation of Compound 4-5

To a solution of compound 4-4 (50 mg, 0.095 mmol) in dioxane (2 mL) were added Pin₂B₂ (49 mg, 0.190 mmol), KOAc (27.97 mg, 0.285 mmol) and Pd(dppf)Cl₂ (6.20 mg, 0.009 mmol). The mixture was stirred at 90° C. under N₂ for 1.5 h. The reaction mixture was cooled to room temperature, diluted with water (10 mL), extracted with EA (20 mL×3), washed with brine (80 mL), dried over Na₂SO₄, filtered, and concentrated to dryness. The crude 4-5 was used directly in next step. LCMS: 574.0 [M+H]⁺.

### Step 5: Preparation of Compound 4-6

To a solution of compound 4-5 (54 mg, 0.009 mmol) in dioxane (2 mL) and H₂O (0.3 mL) was added compound 1-3 (28 mg, 0.141 mmol), K₂CO₃ (39 mg, 0.282 mmol) and Pd(dtbpf)Cl₂ (6.14 mg, 0.009 mmol). The mixture was stirred at 90° C. under N₂ for 1.5 h. The mixture was filtered. The filtrate was concentrated to afford crude compound 4-6, which could be used without further purification. LCMS: 568.0 [M+H]⁺.

### Step 5: Preparation of Example 4

To a solution of compound 4-6 (80 mg, 0.141 mmol) in EtOH (2 mL) was added NaOH (1.4 mL, 1 M in water). The mixture was stirred at 60° C. under N₂ for 5 h. The reaction mixture was concentrated to dryness. The residue was purified by prep-HPLC to afford example 4. LCMS: 414.2 [M+H]⁺. ¹H NMR: (400 MHz, DMSO-d₆) δ 12.04 (s, 1H), 9.12 (s, 1H), 8.71 (d, J=2.4 Hz, 1H), 8.16 (s, 1H), 8.06 (dd, J=8.9, 2.5 Hz, 1H), 7.85 (d, J=2.0 Hz, 1H), 7.58 (d, J=1.9 Hz, 1H), 6.94 (dd, J=11.2, 8.7 Hz, 2H), 6.76 (dd, J=11.2, 5.1 Hz, 2H), 3.60-3.53 (m, 4H), 2.40-2.45 (m, 4H), 2.23 (s, 3H), 1.88 (s, 3H), 1.81 (s, 3H).

### Example 5: Preparation of 2,4-dimethyl-3-(2-(6-methylpyridin-3-yl)-1H-pyrrolo[2,3-b]pyridin-5-yl)phenol

The title compound was prepared according to the following synthetic procedures.

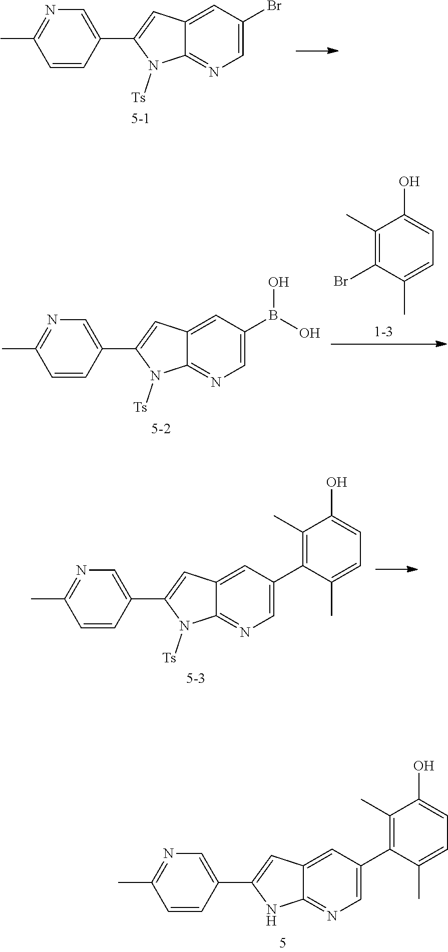

### Step 1: Preparation of Compound 5-2

To a solution of compound 5-1 (150 mg, 0.339 mmol) in dioxane (6 mL) were added 4,4,5,5-tetramethyl-2-(4,4,5,5-tetramethyl-1,3,2-dioxaborolan-2-yl)-1,3,2-dioxaborolane (172 mg, 0.678 mmol), Pd(dppf)Cl₂ (25 mg, 0.034 mmol) and potassium acetate (100 mg, 1.017 mmol). The reaction mixture was stirred at 100 [symbol] . for 2 h. The mixture was cooled to room temperature, diluted with water (20 mL), extracted with EA (30 mL×3), washed with brine (100 mL), dried over Na₂SO₄, filtered, and concentrated to dryness. The crude compound 5-2 was used directly in next step. LCMS: 408.2 [M+H]⁺.

### Step 2: Preparation of Compound 5-3

To a solution of compound 5-2 (150 mg, 0.306 mmol) in dioxane (6 mL) and H₂O (1 mL) were added compound 1-3 (62 mg, 0.306 mmol), K₂CO₃ (127 mg, 0.919 mmol) and Pd(dtbpf)Cl₂ (20 mg, 0.031 mmol). The reaction mixture was stirred at 90 [symbol] . for 2 h. The reaction was diluted with DCM and water. The organic layer was separated and concentrated in vacuo. The crude material was purified by silica gel column to afford compound 5-3. LCMS: 484.2 [M+H]⁺.

### Step 3: Preparation of Example 5

To a solution of compound 5-3 (120 mg, 0.248 mmol) in EtOH (4 mL) and H₂O (2 mL) was added NaOH (99 mg, 2.481 mmol). The reaction mixture was stirred at 80° C. for 1 h. The reaction was diluted with DCM and water. The organic layer was separated and concentrated in vacuo. The organic layer was collected, concentrated in vacuo. The crude product was purified by prep-HPLC to afford example 5. LCMS: 330.1 [M+H]⁺. ¹H NMR: (400 MHz, DMSO-d₆) δ 12.27 (s, 1H), 9.16 (s, 1H), 9.05-9.04 (m, iH), 8.22-8.19 (m, iH), 7.94 (d, J=2.0 Hz, 1H), 7.68 (d, J=1.9 Hz, 1H), 7.37 (d, J=8.1 Hz, 1H), 7.00 (d, J=4.0 Hz, 1H), 6.93 (d, J=8.0 Hz, 1H), 6.76 (d, J=8.2 Hz, 1H), 2.51 (s, 3H), 1.89 (s, 3H), 1.82 (s, 3H).

### Example 6: Preparation of 1-(4-(5-(3-hydroxy-2,6-dimethylphenyl)-1H-pyrrolo[2,3-b]pyridin-2-yl)-3,6-dihydropyridin-1 (2H)-yl)ethan-1-one

The title compound was prepared according to the following synthetic procedures.

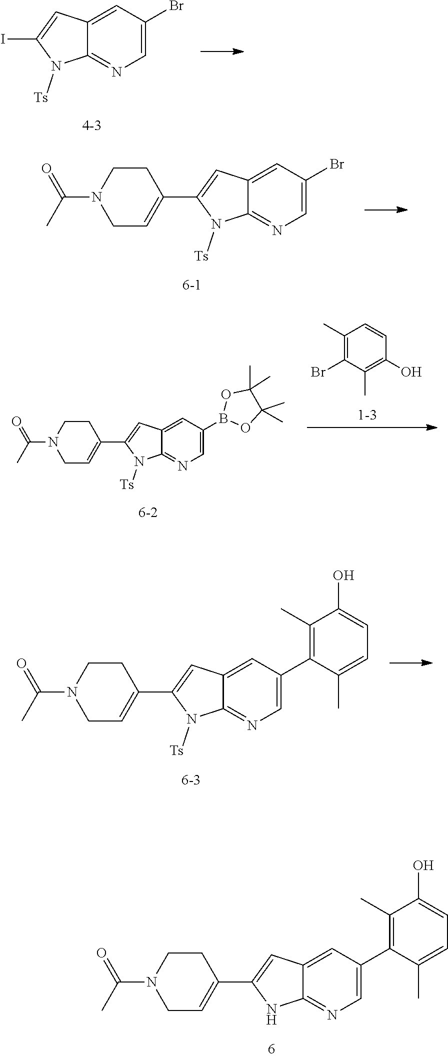

### Step 1: Preparation of Compound 6-1

To a solution of compound 4-3 and 1-[4-(4,4,5,5-tetramethyl-1,3,2-dioxaborolan-2-yl)-1,2,3,6-tetrahydropyridin-1-yl]ethan-1-one (579 mg, 2.306 mmol) in dioxane (10 mL) and H₂O (2 mL) were added Pd(dppf)Cl₂ (153 mg, 0.209 mmol) and cesium fluoride (957 mg, 6.300 mmol). The mixture was stirred at 100° C. for 1 h under N₂. The reaction mixture was cooled to room temperature and extracted with EA (20 mL×2). The organic layer was combined, dried over Na₂SO₄, and filtered. The filtrate was concentrated and purified by silica gel column chromatography to afford compound 6-1. LCMS: 448.9 [M+H]⁺.

### Step 3: Preparation of Compound 6-2

To a solution of compound 6-1 (350 mg, 0.738 mmol) and 4,4,5,5-tetramethyl-2-(4,4,5,5-tetramethyl-1,3,2-dioxaborolan-2-yl)-1,3,2-dioxaborolane (224 mg, 0.885 mmol) in dioxane (3 mL) were added KOAc (217 mg, 2.213 mmol) and Pd(dppf)Cl₂ (54 mg, 0.074 mmol). The mixture was stirred at 100° C. for 1 h. The mixture was cooled to room temperature, diluted with water (10 mL), extracted with EA (20 mL×3), washed with brine (80 mL), dried over Na₂SO₄, filtered, and concentrated to dryness. The crude compound 6-2 was used directly in next step. LCMS: 522.3 [M+H]⁺.

### Step 4: Preparation of Compound 6-3

To a solution of compound 6-2 (350 mg, 0.671 mmol) and compound 1-3 (135 mg, 0.671 mmol) in dioxane (5 mL) and H₂O (1 mL) was added Pd(dtbpf)Cl₂ (44 mg, 0.067 mmol) and K₂CO₃ (278 mg, 2.014 mmol). The mixture was stirred at 90 [symbol] for 2 h. The mixture was extracted with EA (10 mL). The organic layer was dried over Na₂SO₄, filtered, concentrated, and purified by silica gel column chromatography to afford compound 6-3. LCMS: 516.3 [M+H]⁺.

### Step 5: Preparation of Example 6

To a solution of compound 6-3 (100 mg, 0.194 mmol) in EtOH (2 mL) and H₂O (1 mL) was added sodium hydroxide (8.0 mg, 0.194 mmol). The mixture was stirred at 80° C. for 1 h. The reaction mixture was cooled to room temperature, diluted with EA (30 mL), washed with brine (30 mL). The organic layer was dried over Na₂SO₄, filtered, and concentrated to dryness. The residue was purified by prep-HPLC to afford example 6. LCMS: 362.4 [M+H]⁺. ¹H NMR: (400 MHz, DMSO-d₆) δ 11.81 (d, J=12.8 Hz, 1H), 9.12 (s, 1H), 7.88 (s, 1H), 7.60 (s, 1H), 6.91 (d, J=8.2 Hz, 1H), 6.74 (d, J=8.2 Hz, 1H), 6.52 (s, 1H), 6.47 (d, J=5.1 Hz, 1H), 4.21 (s, 1H), 4.15 (s, 1H), 3.65 (s, 2H), 2.60 (s, 2H), 2.07 (d, J=12.6 Hz, 3H), 1.86 (s, 3H), 1.79 (s, 3H).

### Example 7: Preparation of 2,4-dimethyl-3-(2-(2-methylpyrimidin-5-yl)-1H-pyrrolo[2,3-b]pyridin-5-yl)phenol

The title compound was prepared according to the following synthetic procedures.

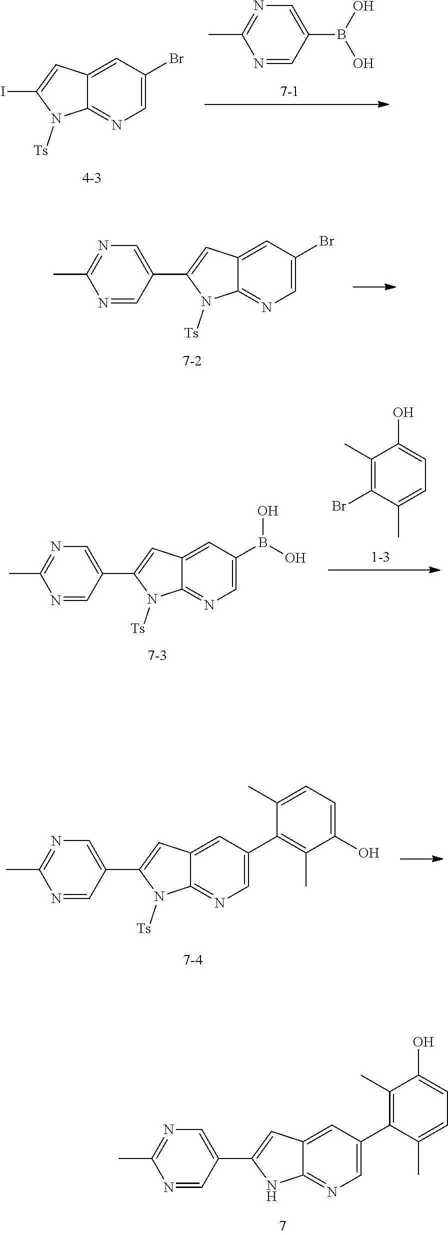

### Step 1: Preparation of Compound 7-2

A solution of compound 4-3 (170 mg, 0.36 mmol), compound 7-1 (49 mg, 0.36 mmol), K₂CO₃ (123 mg, 0.89 mmol) and Pd(dppf)Cl₂ (26 mg, 0.036 mmol) in dioxane (1.0 mL) and water (0.2 mL) was stirred at 100 [symbol] for 2.5 h. The mixture was concentrated. The residue was purified by column chromatography on silica gel to afford compound 7-2 as a yellow solid. LCMS: 445.0 [M+H]⁺.

### Step 2: Preparation of Compound 7-3

A solution of compound 7-2 (100 mg, 0.23 mmol), 4,4,5,5-tetramethyl-2-(4,4,5,5-tetramethyl-1,3,2-dioxaborolan-2-yl)-1,3,2-dioxaborolane (115 mg, 0.45 mmol), KOAc (67 mg, 0.68 mmol) and Pd(dppf)Cl₂ (17 mg, 0.023 mmol) in dioxane (2 mL) was stirred at 100 [symbol] for 16 h. The mixture was cooled to room temperature, diluted with water (10 mL), extracted with EA (20 mL×3), washed with brine (80 mL), dried over Na₂SO₄, filtered, and concentrated to dryness. The crude compound 7-3 was used for the next step without further purification. LCMS: 409.1 [M+H]⁺.

### Step 3: Preparation of Compound 7-4

A solution of compound 7-3, compound 1-3 (50 mg, 0.25 mmol), K₂CO₃ (78 mg, 0.57 mmol) and Pd(dtbpf)Cl₂ (15 mg, 0.023 mmol) in dioxane/water (2 mL/0.2 mL) was stirred at 100 [symbol] for 4 h. The mixture was concentrated. The residue was purified by column chromatography on silica gel to afford compound 7-4. LCMS: 485.2 [M+H]⁺.

### Step 4: Preparation of Example 7

To a solution of compound 7-4 (122 mg, 0.25 mmol) in EtOH (5 mL) was added NaOH (2 M, 1.26 mL, 2.52 mmol). The mixture was stirred at 70 [symbol] for 16 h. The mixture was concentrated, diluted with water (2 mL), neutralized to pH ˜5 with HCl (2 M) solution and concentrated. The crude product was purified by prep-HPLC to afford example 7. LCMS: 331.2 [M+H]⁺. ¹H NMR: (400 MHz, DMSO-d) δ 12.39 (s, 1H), 9.25 (s, 2H), 9.15 (s, 1H), 7.98 (d, J=1.9 Hz, 1H), 7.72 (d, J=1.9 Hz, 1H), 7.12 (s, 1H), 6.93 (d, J=8.2 Hz, 1H), 6.76 (d, J=8.1 Hz, 1H), 2.67 (s, 3H), 1.88 (s, 3H), 1.81 (s, 3H).

### Example 8: Preparation of 3-(2-(5-methoxypyridin-3-yl)-1H-pyrrolo[2,3-b]pyridin-5-yl)-2,4-dimethylphenol

The title compound was prepared according to the following synthetic procedures.

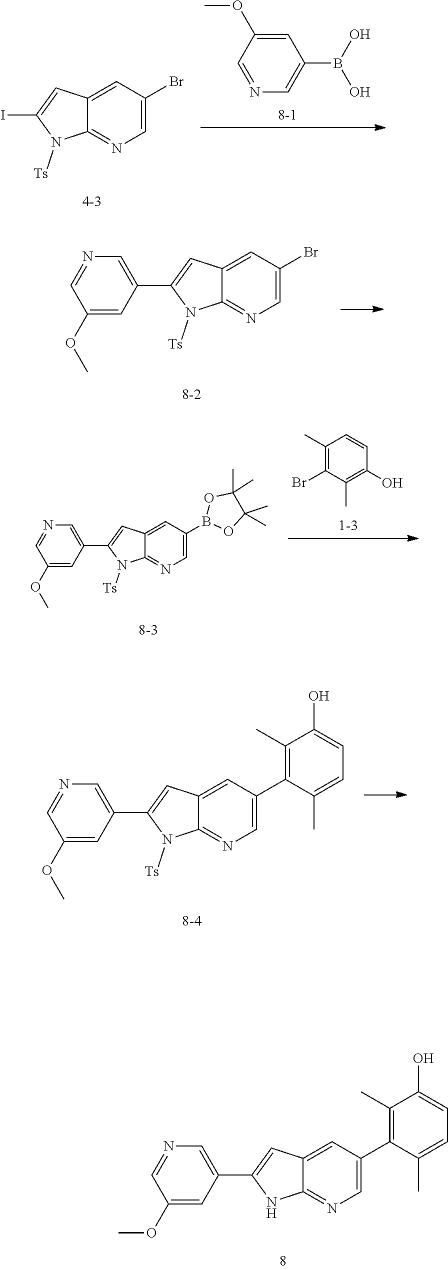

### Step 1: Preparation of Compound 8-2

To a stirred solution of compound 4-3 (160 mg, 0.335 mmol) in dioxane (4 mL) were added Pd(dppf)Cl₂ (24.54 mg, 0.034 mmol), compound 8-1 (61.55 mg, 0.402 mmol), potassium carbonate (139.04 mg, 1.006 mmol) and H₂O (0.8 mL). The reaction was stirred at 100 [symbol] for 2 h by microwave under argon and quenched with water (25 mL). The resulting mixture was extracted with EA (3×20 mL). The EA layer was combined, washed with brine (2×30 mL), dried over Na₂SO₄, filtered and concentrated. The residue was purified by silica gel column chromatography to compound 8-2. LCMS: 457.9 [M+H]⁺.

### Step 2: Preparation of Compound 8-3

To a stirred solution of compound 8-2 (80 mg, 0.175 mmol) in dioxane (2 mL) were added 4,4,5,5-tetramethyl-2-(4,4,5,5-tetramethyl-1,3,2-dioxaborolan-2-yl)-1,3,2-dioxaborolane (66.49 mg, 0.262 mmol), Pd(dppf)Cl₂ (127.72 mg, 0.175 mmol) and KOAC (51.32 mg, 0.524 mmol). The reaction was stirred at 90 [symbol] for 3 h and quenched with H₂O (10 mL). The resulting mixture was extracted with EA (3×15 mL). The EA layer was combined, washed with brine (2×20 mL), dried over Na₂SO₄, filtered and concentrated. The residue was purified by silica gel column chromatography to afford compound 8-3. LCMS: 506.2 [M+H]⁺.

### Step 4: Preparation of Compound 8-4

To a stirred solution of compound 8-3 (70 mg, 0.139 mmol) in dioxane (2 mL) was added compound 1-3 (27.85 mg, 0.139 mmol), Cs₂CO₃ (173.19 mg, 0.532 mmol), Pd(dtbpf)Cl₂ (9.14 mg, 0.014 mmol) and H₂O (0.4 mL). The reaction was stirred at 90′C for 2 h under argon. The reaction mixture was quenched with water (15 mL). The resulting mixture was extracted with EA (3×15 mL). The EA layer was combined, washed with brine (2×20 mL), dried over Na₂SO₄, filtered and concentrated. The residue was purified by silica gel column chromatography to afford compound 8-4. LCMS: 500.2 [M+H]⁺.

### Step 5: Preparation of Example 8

To a stirred solution of compound 8-4 (60 mg, 0.120 mmol) in EtOH (4 mL) was added sodium hydroxide (4.80 mg, 0.120 mmol). The reaction was stirred at 70 [symbol] by microwave for 2 h. The reaction mixture was quenched with HCl aq. (1 M) to pH-7. The mixture was concentrated to dryness. The residue was purified by prep-HPLC to afford example 8. LCMS: 346.3 [M+H]⁺; ¹H NMR: (400 MHz, DMSO-d) δ 12.33 (s, 1H), 9.16 (s, 1H), 8.79 (d, J=1.6 Hz, 1H), 8.27 (d, J=2.7 Hz, 1H), 8.16 (s, 1H), 7.97 (d, J=2.0 Hz, 1H), 7.96-7.91 (m, 1H), 7.71 (d, J=1.9 Hz, 1H), 7.12 (d, J=1.9 Hz, 1H), 6.94 (d, J=8.2 Hz, 1H), 6.76 (d, J=8.2 Hz, 1H), 3.93 (s, 3H), 1.89 (s, 3H), 1.82 (s, 3H).

### Example 9: Preparation of 4-(5-(3-hydroxy-2,6-dimethylphenyl)-1H-pyrrolo[2,3-b]pyridin-2-yl)-1-methylpyridin-2 (1H)-one

The title compound was prepared according to the following synthetic procedures.

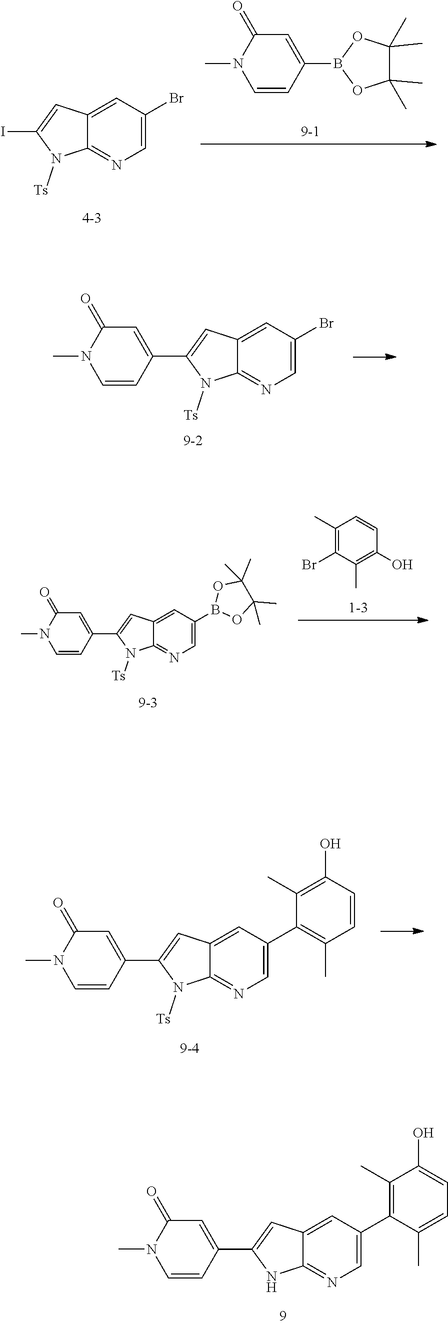

### Step 1: Preparation of Compound 9-2

To a solution of compound 4-3 (380 mg, 0.796 mmol) in dioxane (6 mL) and H₂O (1 mL) was added compound 9-1 (187.24 mg, 0.796 mmol), cesium fluoride (362.95 mg, 2.389 mmol) and Pd(dppf)Cl₂ (58.28 mg, 0.080 mmol). The resulting mixture was stirred at 90 [symbol] under N₂ for 2 h. The reaction mixture was quenched with water (20 mL), and extracted with EA (30 mL×3). The combined organic layer was washed with brine (30 mL), dried over Na₂SO₄, filtered, and concentrated to dryness. The residue was purified using silica gel column chromatography to afford compound 9-2. LCMS: 460.0 [M+H]⁺.

### Step 2: Preparation of Compound 9-3

To a solution of compound 9-2 (200 mg, 0.436 mmol) in dioxane (6 mL) were added 4,4,5,5-tetramethyl-2-(4,4,5,5-tetramethyl-1,3,2-dioxaborolan-2-yl)-1,3,2-dioxaborolane (132.97 mg, 0.524 mmol), KOAc (128.48 mg, 1.309 mmol) and Pd(dppf)Cl₂ (31.93 mg, 0.044 mmol). The reaction was stirred at 100 [symbol] under N₂ for 3 h. The reaction mixture was diluted with water (20 mL), extracted with EA (30 mL×3). The combined organic layer was washed with brine (30 mL), dried over Na₂SO₄, filtered, and concentrated to dryness. The residue was purified by silica gel column chromatography to afford compound 9-3. LCMS: 506.1 [M+H]⁺.

### Step 3: Preparation of Compound 9-4

To a solution of compound 9-3 (200 mg, 0.394 mmol) in dioxane (6 mL) and H₂O (1 mL) were added compound 1-3 (79.25 mg, 0.394 mmol), Pd(dtbpf)Cl₂ (25.77 mg, 0.039 mmol) and K₂CO₃ (163.42 mg, 1.182 mmol). The reaction was stirred at 90° C. under N₂ for 2 h. The reaction mixture was diluted with water (20 mL), extracted with EA (30 mL×3). The combined organic layer was washed with brine (30 mL), dried over Na₂SO₄, filtered, and concentrated to dryness. The residue was purified by silica gel column chromatography to afford compound 9-4. LCMS: 500.2 [M+H]⁺.

### Step 4: Preparation of Example 9

To a solution of compound 9-4 (100 mg, 0.200 mmol) in EtOH (3 mL) and H₂O (3 mL) was added NaOH (80.07 mg, 2.002 mmol). The reaction was stirred at room temperature for 3 h. The reaction was diluted with DCM and ice-water, adjusted to pH ˜7 with HCl (0.5 M in water). The reaction mixture was diluted with water (20 mL), extracted with EA (30 mL×3). The combined organic layer was washed with brine (30 mL), dried over Na₂SO₄, filtered, and concentrated to dryness. The crude product was purified by prep-HPLC to afford example 9. LCMS: 346.1 [M+H]⁺. ¹H NMR: (400 MHz, DMSO-d₆) δ 12.27 (s, 1H), 9.17 (s, 1H), 8.01 (d, J=1.8 Hz, 1H), 7.84-7.64 (m, 2H), 7.17-7.08 (m, 1H), 7.02 (d, J=1.4 Hz, 1H), 6.94 (d, J=8.2 Hz, 1H), 6.82-6.73 (m, 2H), 3.45 (s, 3H), 1.88 (s, 3H), 1.81 (s, 3H).

### Example 10: Preparation of 3-(4-chloro-2-(6-methylpyridin-3-yl)-1H-pyrrolo[2,3-b]pyridin-5-yl)-2,4-dimethylphenol

The title compound was prepared according to the following synthetic procedures.

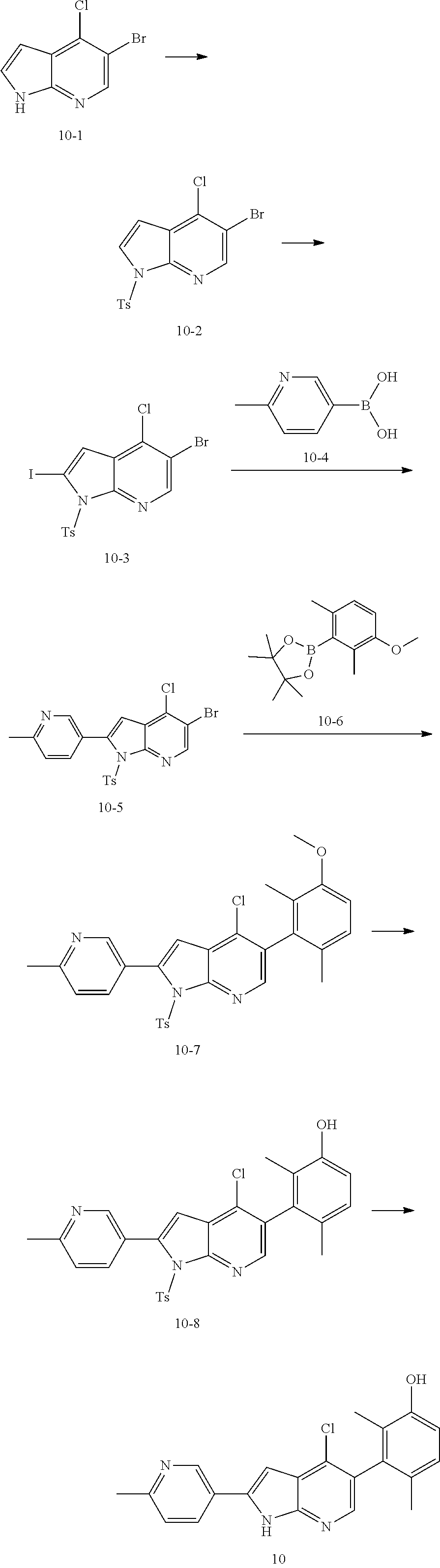

### Step 1: Preparation of Compound 10-2

To a solution of compound 10-1 (950 mg, 4.104 mmol) in THF (20 mL) was added NaH (328.3 mg, 60% dispersion in mineral oil, 8.208 mmol) at 0° C. The mixture was stirred at 0° C. for 0.5 h. Then TsCl (935.72 mg, 4.925 mmol) was added. The mixture was stirred at room temperature for 1 h. The reaction mixture was quenched by sat. NH₄Cl (aq., 20 mL), extracted with EA (150 mL). The organic layers were combined and dried over Na₂SO₄. After filtration, the filtrate was concentrated and purified by silica gel column chromatography to afford compound 10-2. LCMS: 387.0 [M+H]⁺.

### Step 2: Preparation of Compound 10-3

To a solution of compound 10-2 (700 mg, 1.818 mmol) in THF (10 mL) was added LDA (1.09 mL, 2.182 mmol) at −78° C. The mixture was stirred at this temperature for 1 h. Then I₂ (692.18 mg, 2.727 mmol) was added, and the resulting mixture was stirred at −78° C. for 1 h. The mixture was quenched with sat. NH₄Cl (aq., 20 mL), extracted with EA (100 mL). The organic layer was washed with sat. Na₂SO₃ (aq., 30 mL). The organic layer was dried over Na₂SO₄ and concentrated to dryness. The residue was purified by silica gel column chromatography to afford compound 10-3. LCMS: 512.9 [M+H]⁺.

### Step 3: Preparation of Compound 10-5

To a solution of compound 10-3 (250 mg, 0.489 mmol) in dioxane (5 mL) and H₂O (1 mL) was added compound 10-4 (100.38 mg, 0.733 mmol), CsF (222.85 mg, 1.466 mmol) and Pd(dppf)Cl₂ (35.72 mg, 0.049 mmol). The mixture was stirred at 90° C. under N₂ for 6 h. The reaction mixture was filtered. The filtrate was concentrated to dryness. The residue was purified by silica gel column chromatography to afford compound 10-5. LCMS: 477.8 [M+H]⁺.

### Step 4: Preparation of Compound 10-7

To a solution of compound 10-5 (93 mg, 0.195 mmol) in dioxane (5 mL) and H₂O (1 mL) were added compound 10-6 (61.35 mg, 0.234 mmol), Pd(dtbpf)Cl₂ (14.25 mg, 0.020 mmol) and K₂CO₃ (80.85 mg, 0.585 mmol). The mixture was stirred at 90° C. for 2 h. The reaction mixture was filtered. The filtrate was concentrated to dryness. The residue was purified by prep-TLC to afford compound 10-7. LCMS: 532.2 [M+H]⁺.

### Step 5: Preparation of Compound 10-8

To a solution of compound 10-7 (60 mg, 0.113 mmol) in DCM (3 mL) was added BBr₃ (0.1 mL) at 0° C. The mixture was stirred for 0.5 h. The reaction was quenched with MeOH (1 mL). The mixture was concentrated to dryness. The residue was purified by silica gel column chromatography to afford compound 10-8. LCMS: 518.2 [M+H]⁺.

### Step 6: Preparation of Example 10

To a solution of compound 10-8 (40 mg, 0.077 mmol) in EtOH (5 mL) was added NaOH (1.5 mL, 1 M in water). The mixture was stirred at 65° C. for 3 h. The mixture was concentrated to dryness. The residue was purified by prep-HPLC to afford example 10. LCMS: 364.2 [M+H]⁺. ¹H NMR: (400 MHz, DMSO-d₆) δ 12.75 (s, 1H), 9.21 (s, 1H), 8.48 (d, J=7.9 Hz, 1H), 8.04 (s, 1H), 7.59 (d, J=7.9 Hz, 1H), 7.54 (d, J=7.7 Hz, 1H), 7.25 (s, 1H), 7.03 (d, J=7.9 Hz, 1H), 6.86 (d, J=8.1 Hz, 1H), 2.64 (s, 3H), 2.35 (s, 1H), 1.90 (s, 3H), 1.83 (s, 3H).

### Example 11: Preparation of 2,4-dimethyl-3-(6-(6-methylpyridin-3-yl)-5H-pyrrolo[2,3-b]pyrazin-2-yl)phenol

The title compound was prepared according to the following synthetic procedures.

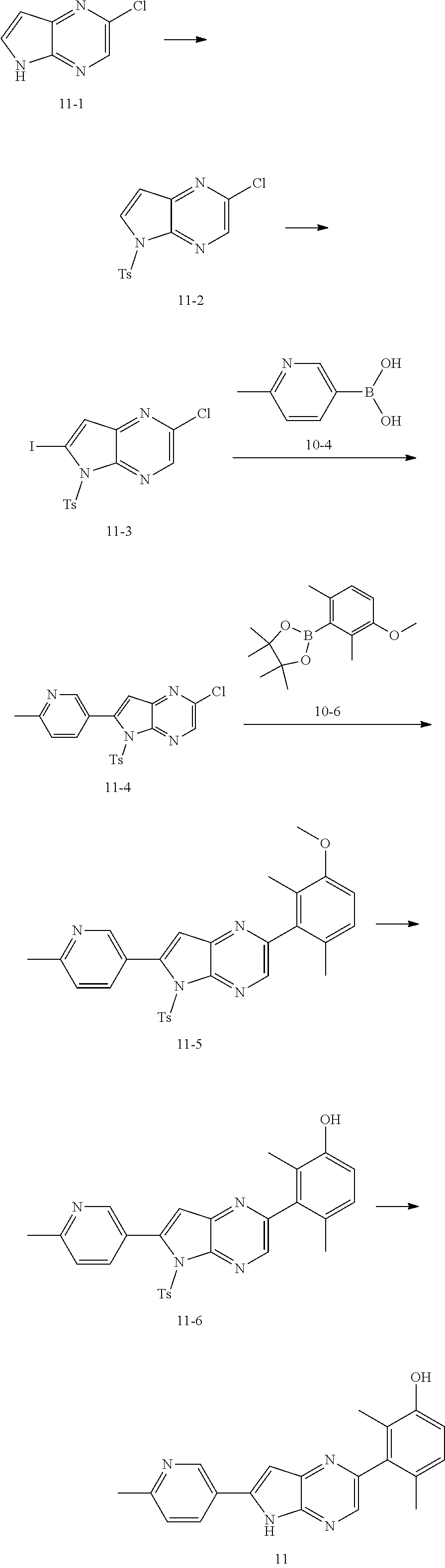

### Step 1: Preparation of Compound 11-2

To a solution of compound 11-1 (500 mg, 3.256 mmol) in THF (10 mL) was added NaH (260.48 mg, 60% dispersion in mineral oil, 6.512 mmol) at 0° C. The mixture was stirred at this temperature for 0.5 h. Then TsCl (744.87 mg, 3.907 mmol) was added. The reaction mixture was stirred at room temperature for 1 h. The mixture was quenched with sat. NH₄Cl (aq., 20 mL), extracted with EA (100 mL). The organic layer was dried over Na₂SO₄ and concentrated to dryness. The residue was purified by silica gel column chromatography to afford compound 11-2. LCMS: 307.9 [M+H]⁺.

### Step 2: Preparation of Compound 11-3

To a solution of compound 11-2 (1.1 g, 3.574 mmol) in THF (20 mL) was added LDA (2.32 mL, 4.647 mmol) at −78° C. The mixture was stirred at this temperature for 1 h. Then 12 (1.36 g, 5.361 mmol) was added and stirred at −78° C. for 1 h. The mixture was quenched with sat. NH₄Cl (aq, 20 mL), extracted with EA (100 mL). The organic layer was washed with Na₂SO₃ (sat.aq, 30 mL), dried over Na₂SO₄ and concentrated to dryness. The residue was purified by silica gel column chromatography to afford compound 11-3. LCMS: 434.0 [M+H]⁺.

### Step 3: Preparation of Compound 11-4

To a solution of compound 11-3 (500 mg, 1.153 mmol) in dioxane (10 mL) and H₂O (2 mL) was added compound 10-4 (236.84 mg, 1.730 mmol), CsF (525.77 mg, 1.730 mmol) and Pd(dppf)Cl₂ (84.28 mg, 0.115 mmol). The mixture was stirred at 90° C. under N₂ overnight. The reaction mixture was filtered. The filtrate was concentrated to dryness. The residue was purified by silica gel column chromatography to afford compound 11-4. LCMS: 399.0 [M+H]⁺.

### Step 4: Preparation of Compound 11-5

To a mixture of compound 11-4 (200 mg, 0.50 mmol) in dioxane (5 mL) and H₂O (1 mL) were added compound 10-6 (157.74 mg, 0.60 mmol), Pd(dtbpf)Cl₂ (32.7 mg, 0.05 mmol) and K₂CO₃ (207.89 mg, 1.504 mmol). The mixture was stirred at 90° C. for 2 h. The reaction mixture was filtered. The filtrate was concentrated to dryness. The residue was purified by silica gel column chromatography to afford compound 11-5. LCMS: 499.2 [M+H]⁺.

### Step 5: Preparation of Compound 11-6

To a solution of compound 11-5 (200 mg, 0.011 mmol) in DCM (10 mL) was added BBr₃ (0.2 mL) at 0° C. The mixture was stirred for 0.5 h. The reaction was quenched with MeOH (1 mL). The mixture was concentrated to dryness. The residue was purified by silica gel column chromatography to afford compound 11-6. LCMS: 485.2 [M+H]⁺.

### Step 6: Preparation of Example 11

To a solution of Compound 7 (120 mg, 0.248 mmol) in EtOH (10 mL) was added NaOH (2.5 mL, 1 M in water). The mixture was stirred at 65° C. for 3 h. The mixture was concentrated to dryness. The residue was purified by prep-HPLC to example 11. LCMS: 331.2 [M+H]⁺. ¹H NMR: (400 MHz, CD₃OD) δ 9.00 (d, J=1.9 Hz, 1H), 8.27 (dd, J=8.1, 2.2 Hz, 1H), 8.15 (s, 1H), 7.47 (d, J=8.2 Hz, 1H), 7.09 (s, 1H), 6.98 (d, J=8.2 Hz, 1H), 6.80 (d, J=8.2 Hz, 1H), 2.62 (s, 3H), 1.94 (s, 3H), 1.89 (s, 3H).

### Example 12: Preparation of 3-(7-fluoro-6-(2-methylpyrimidin-5-yl)-5H-pyrrolo[2,3-b]pyrazin-2-yl)-2,4-dimethylphenol

The title compound was prepared according to the following synthetic procedures.

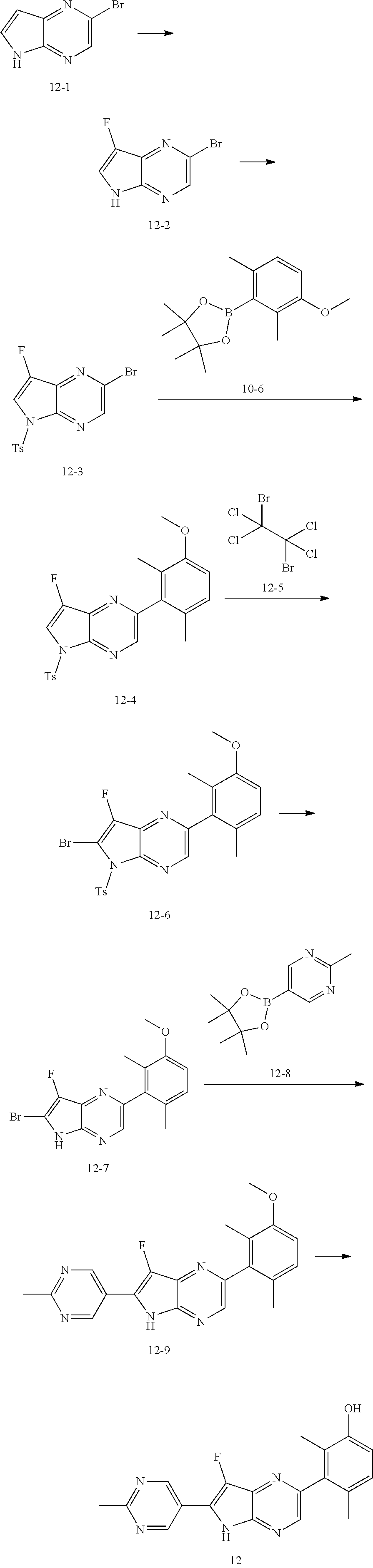

### Step 1: Preparation of Compound 12-2

To a solution of compound 12-1 (500 mg, 2.525 mmol) in HOAc (2 mL) and CH₃CN (10 mL) was added selectfluor (1341.8 mg, 3.787 mmol). The solution was heated to 90 [symbol] for 16 h under N₂. The solution was quenched by sat.aq NaHCO₃ (50 mL), extracted with EA (50 mL×3). Combined organic layers were washed with brine, dried over Na₂SO₄, filtered and concentrated. The residue was purified by column chromatography to afford compound 12-2 (100 mg, 0.463 mmol, 18.33%). LCMS: 215.9 [M+H]⁺.

### Step 2: Preparation of Compound 12-3

To a solution of compound 12-2 (100 mg, 0.463 mmol) in DMF (2 mL) was added NaH (27.8 mg, 0.694 mmol) in portions at 0 [symbol] The mixture was stirred at 0 [symbol] for 30 min. Then TsCl (132.4 mg, 0.694 mmol) was added. The reaction mixture was stirred at 0 [symbol] for 1 h and quenched by H₂O (10 mL). The suspension was filtered and the solid was dried to afford compound 12-3 (150 mg, 0.405 mmol, 87.52%). LCMS: 370.1 [M+H]⁺.

### Step 3: Preparation of Compound 12-4

A mixture of compound 12-3 (150 mg, 0.405 mmol), compound 10-6 (138.1 mg, 0.527 mmol), K₂CO₃ (168.0 mg, 1.216 mmol) and Pd(dtbpf)Cl₂ (26.2 mg, 0.041 mmol) in dioxane-water (5:1) (5 mL) was heated to 90 [symbol] for 2 h under N₂. The mixture was diluted with H₂O (10 mL), extracted with EA (10 mL×3). The combined organic layer was washed with brine, dried over Na₂SO₄, filtered and concentrated. The residue was purified by column chromatography on silica gel to afford compound 12-4 (100 mg, 0.235 mmol, 58.00%). LCMS: 426.0 [M+H]⁺.

### Step 4: Preparation of Compound 12-6

To a solution of compound 12-4 (95 mg, 0.223 mmol) in THF (4 mL) was added LDA (0.22 mL, 0.447 mmol) in drops at −78 [symbol] . The solution was stirred at −78 [symbol] for 30 min. Then compound 12-5 (145.4 mg, 0.447 mmol) in THF (0.4 ml) was added. The reaction mixture was stirred at −78 [symbol] for 1 h under N₂. The mixture was quenched by sat.aq NH₄Cl (20 mL), diluted with H₂O (20 mL), extracted with EA (30 mL×3). Combined organic layers were washed with brine, dried over Na₂SO₄, filtered and concentrated. The residue was purified by column chromatography on silica gel to afford compound 12-6 (90 mg, 0.178 mmol, 79.92%). LCMS: 505.6 [M+H]⁺.

### Step 5: Preparation of Compound 12-7

A solution of compound 12-6 (200 mg, 0.397 mmol) and NaOH (4.0 mL, 3.965 mmol) in EtOH (4 mL) was heated to 50 [symbol] for 1 h. The mixture was cooled to 0 [symbol] , adjusted pH to 6 by 1N HCl, diluted with H₂O (10 mL), extracted with EA (10 mL×3). Combined organic layers were washed with brine, dried over Na₂SO₄, filtered and concentrated. The residue was purified by column chromatography on silica gel to afford compound 12-7. LCMS: 351.7 [M+H]⁺.

### Step 6: Preparation of Compound 12-9

A mixture of compound 12-7 (100 mg, 0.286 mmol), compound 12-8 (81.7 mg, 0.371 mmol), K₂CO₃ (118.4 mg, 0.857 mmol) and Pd(dtbpf)Cl₂ (18.7 mg, 0.029 mmol) in dioxane-water (5:1) (2 mL) was heated to 90 [symbol] for 3 h under N₂ atmosphere. The mixture was concentrated and the residue was diluted with H₂O (10 mL), extracted with EA (10 mL×3). Combined organic layers were washed with brine, dried over Na₂SO₄, filtered and concentrated. The residue was purified by column chromatography on silica gel to afford compound 12-9.

LCMS: 364.1 [M+H]⁺.

### Step 7: Preparation of Example 12

To a solution of compound 12-9 (50 mg, 0.138 mmol) in DCM (4 mL) was added BBr₃ (344.7 mg, 1.376 mmol) in drops at 0 [symbol] . The mixture was stirred at rt for 1 h. The mixture was quenched by MeOH (2 mL) and concentrated. The residue was purified by prep-HPLC to afford example 12. LCMS: 350.0 [M+H]⁺. 1H NMR: (400 MHz, DMSO-d₆) δ 12.76 (s, 1H), 9.26 (s, 1H), 9.23 (s, 2H), 8.27 (s, 1H), 6.96 (d, J=8.2 Hz, 1H), 6.82 (d, J=8.2 Hz, 1H), 2.72 (s, 3H), 1.88 (s, 3H), 1.80 (s, 3H).

### Example 13: Preparation of 2,4-dimethyl-3-(7-methyl-6-(2-methylpyrimidin-5-yl)-5H-pyrrolo[2,3-b]pyrazin-2-yl)phenol

The example 13 was synthesized from compound 13-1, which was prepared according to the methods described in example 12 with analogous starting material.

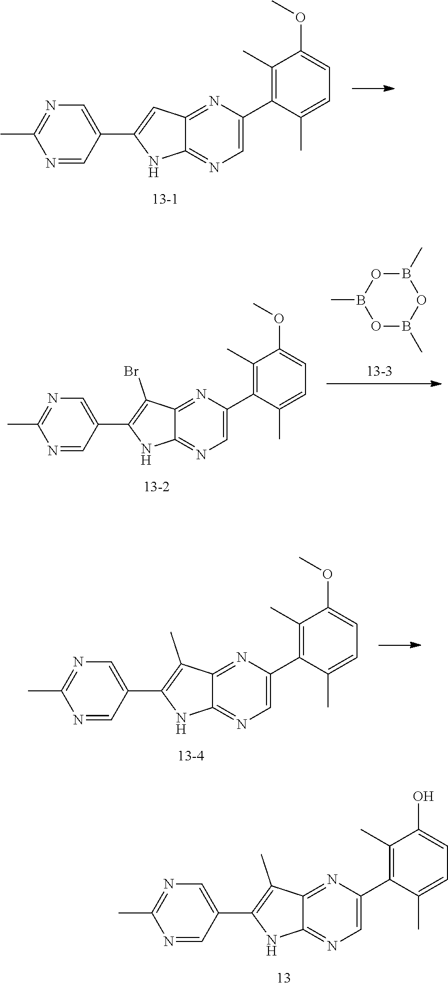

### Step 1: Preparation of Compound 13-2

To a solution of compound 13-1 (200.0 mg, 0.579 mmol) in CH₃CN (10.0 mL) was added NBS (103.1 mg, 0.579 mmol) at 0 [symbol] . The mixture was stirred at room temperature for 2 h. The reaction mixture was purified by reversed-phase column chromatography to afford compound 13-2. LCMS: 426.0 [M+H]⁺.

### Step 2: Preparation of Compound 13-4

A mixture of compound 13-2 (243.0 mg, 0.573 mmol), compound 13-3 (151.0 mg, 1.203 mmol), K₂CO₃ (336.4 mg, 2.434 mmol) and Pd(dtbpf)Cl₂ (37.4 mg, 0.057 mmol) in dioxane-water (6 mL, 5:1) were stirred at 100° C. under N₂ for 16 h. The reaction mixture was cooled to room temperature and diluted with water (20 mL), which was extracted with EA (30 mL×3). The combined organic layer was washed with brine (30 mL), dried over Na₂SO₄, filtered and concentrated. The residue was purified by silica gel column chromatography to afford compound 13-4. LCMS: 360.2 [M+H]⁺.

### Step 3: Preparation of Example 13

To a solution of compound 13-4 (242.0 mg, 0.673 mmol) in DCM (10.0 mL) was added BBr₃ (1.69 mg, 6.733 mmol) at 0 [symbol] . The mixture was stirred at 0 [symbol] for 1 h. The reaction mixture was carefully added into MeOH (5 mL) while stirring and was diluted with water (20 mL), which was adjusted to pH=8 with NaHCO₃ and extracted with EA (30 mL×3). The combined organic layer was washed with brine (30 mL), dried over Na₂SO₄, filtered, and concentrated to dryness. The residue was purified by prep-HPLC to afford example 13. LCMS: 346.1 [M+H]⁺; ¹H NMR: (400 MHz, DMSO-d₆) δ 12.41 (s, 1H), 9.23 (s, 1H), 9.11 (d, J=2.8 Hz, 2H), 8.15 (d, J=2.8 Hz, 1H), 6.95 (d, J=8.0 Hz, 1H), 6.84-6.74 (m, 1H), 2.72 (s, 3H), 2.47 (d, J=2.8 Hz, 3H), 1.88 (s, 3H), 1.80 (s, 3H).

### Example 14: Preparation of 3-(6-(2-aminopyrimidin-5-yl)-7-(3-hydroxy-3-methylbut-1-yn-1-yl)-5H-pyrrolo[2,3-b]pyrazin-2-yl)-2,4-dimethylphenol

The example 14 was synthesized from compound 14-1, which was prepared according to the methods described in example 12 with analogous starting material.

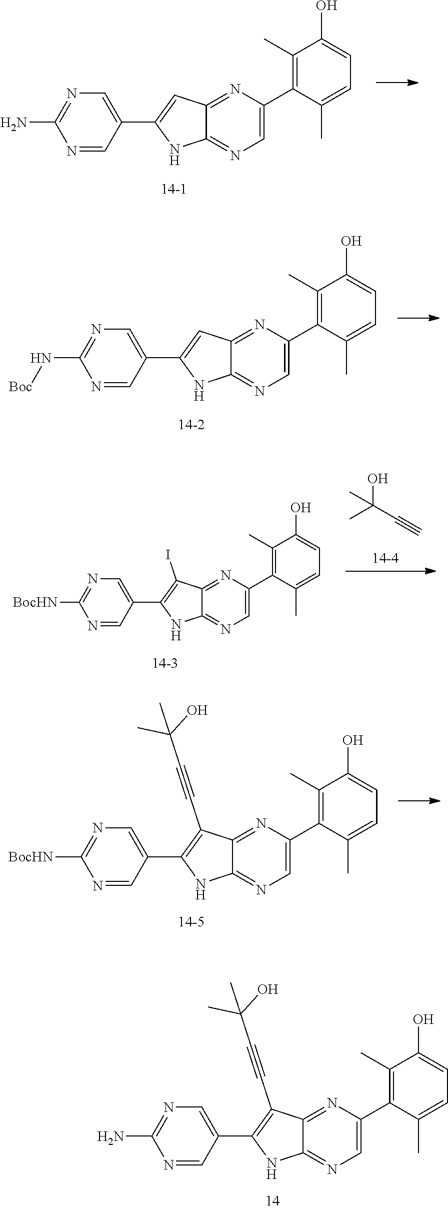

### Step 1: Preparation of Compound 14-2

To a solution of compound 14-1 (320 mg, 0.963 mmol) in DMF (2 mL) was added Boc₂O (0.2 mL, 1.059 mmol) and TEA (0.2 mL, 1.444 mmol) and DMAP (6 mg, 0.048 mmol). The mixture was stirred at 60° C. for 2 h. The reaction mixture was diluted with EA (10 mL), which was washed with brine (10 mL×3). The organic layer was dried over Na₂SO₄, filtered, and concentrated to dryness. The residue was purified by silica gel column chromatography to afford compound 14-2 (255 mg, 0.590 mmol, 61.2%). LCMS: 433.4 [M+H]⁺.

### Step 2: Preparation of Compound 14-3

To a solution of compound 14-2 (260 mg, 0.509 mmol) in CAN (10 mL) was added NIS (103 mg, 0.458 mmol). The mixture was stirred at 25° C. for 2 h. The reaction mixture was diluted with water (10 mL), which was extracted with EA (10 mL×3). The combined organic layer was washed with brine (20 mL), dried over Na₂SO₄, filtered, and concentrated to dryness. The residue was purified by silica gel column chromatography to afford compound 14-3. LCMS: 559.3 [M+H]⁺.

### Step 3: Preparation of Compound 14-5

To a solution of compound 14-3 (250 mg, 0.518 mmol) in TEA (0.9 mL) and DMF (0.3 mL) were added compound 14-4 (51 mg, 0.518 mmol), CuI (40 mg, 0.207 mmol) and PdCl₂(PPh₃)₂ (81 mg, 0.104 mmol). The mixture was stirred at 40° C. for 2 h under N₂. The solution was purified by prep-HPLC to afford compound 14-5. LCMS: 515.4 [M+H]⁺.

### Step 4: Preparation of Example 14

To a solution of compound 14-5 (35 mg, 0.068 mmol) in MeOH (2 mL) was added K₂CO₃ (28 mg, 0.204 mmol) The mixture was stirred at 70° C. for 2 h under N₂. The reaction mixture was diluted with water (5 mL), which was extracted with EA (5 mL×3). The combined organic layer was washed with brine (10 mL), dried over Na₂SO₄, filtered, and concentrated to dryness. The residue was purified by prep-TLC to afford example 14. LCMS: 415.1 [M+H]⁺; ¹H NMR: (400 MHz, DMSO-d₆) δ 12.68 (s, 1H), 9.24 (s, 1H), 9.07 (s, 2H), 8.11 (s, 1H), 7.27 (s, 1H), 6.95 (d, J=8.4 Hz, 1H), 6.81 (d, J=8.4 Hz, 1H), 5.48 (s, 1H), 1.86 (s, 3H), 1.78 (s, 3H), 1.48 (s, 6H).

The following examples 15-27 were prepared according to the methods described in examples 12-14 with analogous starting materials.

| | | LCMS | |
|---|---|---|---|
| Example | Structure | [M + H]⁺ | ¹H NMR |
| 15 | 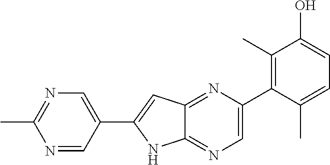 [SMILES: Cc1ncc(-c2cc3nc(-c4c(C)ccc(O)c4C)cnc3[nH]2)cn1] | 332.1 | (400 MHz, DMSO-d₆) δ 12.77 (s, 1H), 9.33 (s, 2H), 9.24 (s, 1H), 8.14 (s, 1H), 7.37 (s, 1H), 6.95 (d, J = 8.0 Hz, 1H), 6.80 (d, J = 8.0 Hz, 1H), 2.70 (s, 3H), 1.87 (s, 3H), 1.80 (s, 3H). |
| | 2,4-dimethyl-3-(6-(2- | | |
| | methylpyrimidin-5-yl)-5H- | | |
| | pyrrolo[2,3-b]pyrazin-2-yl)phenol | | |
| 16 | 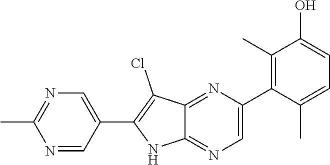 [SMILES: Cc1ncc(-c2[nH]c3ncc(-c4c(C)ccc(O)c4C)nc3c2Cl)cn1] | 366.1 | (400 MHz, DMSO-d₆) δ 13.10 (s, 1H), 9.26 (s, 3H), 8.30 (s, 1H), 6.97 (d, J = 8.0 Hz, 1H), 6.83 (d, J = 8.0 Hz, 1H), 2.74 (s, 3H), 1.88 (s, 3H), 1.80 (s, 3H). |
| | 3-(7-chloro-6-(2-methylpyrimidin- | | |
| | 5-y1)-5H-pyrrolo[2,3-b]pyrazin-2- | | |
| | yl)-2,4-dimethylphenol | | |
| 17 | 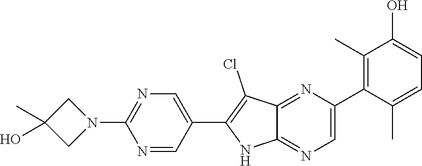 [SMILES: Cc1ccc(O)c(C)c1-c1cnc2[nH]c(-c3cnc(N4CC(C)(O)C4)nc3)c(Cl)c2n1] | 437.1 | (400 MHz, DMSO-d₆) δ 12.89 (s, 1H), 9.32 (s, 1H), 8.92 (s, 1H), 8.46 (s, 1H), 8.17 (s, 1H), 6.95 (d, J = 8.0 Hz, 1H), 6.81 (d, J = 8.0 Hz, 1H), 5.75 (s, 1H), 4.04-3.97 (m, 4H), 1.87 (s, 3H), 1.79 (s, 3H), 1.47 (s, 3H) |
| | 1-(5-(7-chloro-2-(3-hydroxy-2,6- | | |
| | dimethylphenyl)-5H-pyrrolo[2,3- | | |
| | b]pyrazin-6-yl)pyrimidin-2-yl)-3- | | |
| | methylazetidin-3-ol | | |
| 18 | 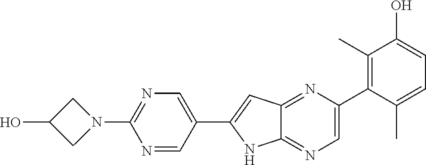 [SMILES: Cc1ccc(O)c(C)c1-c1cnc2[nH]c(-c3cnc(N4CC(O)C4)nc3)cc2n1] | 389.4 | (400 MHz, DMSO-d₆) δ 12.45 (s, 1H), 9.19 (s, 1H), 8.98 (s, 2H), 8.01 (s, 1H), 7.05 (s, 1H), 6.93 (d, J = 8.0 Hz, 1H), 6.79 (d, J = 8.0 Hz, 1H), 5.77 (d, J = 5.6 Hz, 1H), 4.60 (s, 1H), 4.33 (dd, J = 9.4, 6.8 Hz, 2H), 3.87 (dd, J = 9.8, 4.4 Hz, 2H), 1.87 (s, 3H), 1.79 (s, 3H). |
| | 1-(5-(2-(3-hydroxy-2,6- | | |
| | dimethylphenyl)-5H-pyrrolo[2,3- | | |
| | b]pyrazin-6-yl)pyrimidin-2- | | |
| | yl)azetidin-3-ol | | |
| 19 | 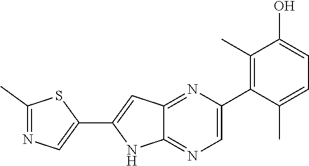 [SMILES: Cc1ncc(-c2cc3nc(-c4c(C)ccc(O)c4C)cnc3[nH]2)s1] | 337.1 | (400 MHz, DMSO-d₆) δ 12.71 (s, 1H), 9.24 (s, 1H), 8.28 (s, 1H), 8.08 (s, 1H), 6.99-6.85 (m, 2H), 6.79 (d, J = 8.2 Hz, 1H), 2.73 (s, 3H), 1.86 (s, 3H), 1.78 (s, 3H). |
| | 2,4-dimethyl-3-(6-(2- | | |
| | methylthiazol-5-y1)-5H- | | |
| | pyrrolo[2,3-b]pyrazin-2-yl)phenol | | |
| 20 | 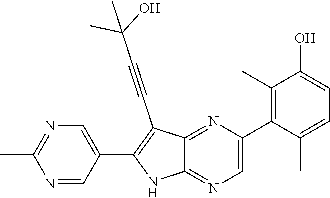 [SMILES: Cc1ncc(-c2[nH]c3ncc(-c4c(C)ccc(O)c4C)nc3c2C#CC(C)(C)O)cn1] | 414.3 | (400 MHz, DMSO-d₆) δ 13.07 (s, 1H), 9.46 (s, 2H), 9.27 (s, 1H), 8.24 (s, 1H), 6.96 (d, J = 8.4 Hz, 1H), 6.82 (d, J = 8.0 Hz, 1H), 5.53 (s, 1H), 2.73 (s, 3H), 1.87 (s, 3H), 1.79 (s, 3H), 1.49 (s, 6H) |
| | 3-(7-(3-hydroxy-3-methylbut-1- | | |
| | yn-1-yl)-6-(2-methylpyrimidin-5- | | |
| | yl)-5H-pyrrolo[2,3-b]pyrazin-2- | | |
| | yl)-2,4-dimethylphenol | | |
| 21 | 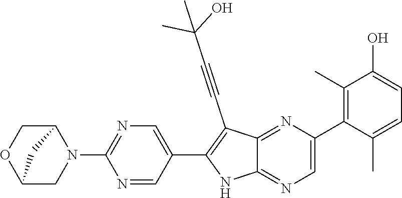 [SMILES: Cc1ccc(O)c(C)c1-c1cnc2[nH]c(-c3cnc(N4C[C@@H]5C[C@H]4CO5)nc3)c(C#CC(C)(C)O)c2n1] | 497.1 | (400 MHz, DMSO-d₆) δ 12.74 (s, 1H), 9.23 (s, 1H), 9.16 (s, 2H), 8.12 (s, 1H), 6.95 (d, J = 8.0 Hz, 1H), 6.81 (d, J = 8.0 Hz, 1H), 5.48 (s, 1H), 5.07 (s, 1H), 4.72 (s, 1H), 3.85 (d, J = 7.2 Hz, 1H), 3.73 (d, J = 7.2 Hz, 1H), 3.58 (d, J = 11.0 Hz, 1H), 3.48 (d, J = 11.0 Hz, 1H), 1.87 (s, 3H), 1.79 (s, 3H), 1.48 (s, 6H), 1.24 (s, 2H). |
| | 3-(6-(2-((1S,4S)-2-oxa-5- | | |
| | azabicyclo[2.2.1]heptan-5- | | |
| | yl)pyrimidin-5-yl)-7-(3-hydroxy-3- | | |
| | methylbut-1-yn-1-yl)-5H- | | |
| | pyrrolo[2,3-b]pyrazin-2-yl)-2,4- | | |
| | dimethylphenol | | |
| 22 | 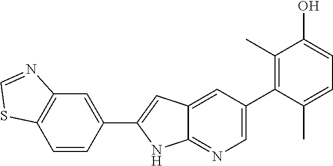 [SMILES: Cc1ccc(O)c(C)c1-c1cnc2[nH]c(-c3ccc4scnc4c3)cc2c1] | 372.1 | (400 MHz, DMSO-d₆) δ 12.31 (s, 1H), 9.46 (s, 1H), 9.15 (s, 1H), 8.71 (d, J = 1.6 Hz, 1H), 8.27 (d, J = 8.4 Hz, 1H), 8.09 (dd, J = 8.4, 1.6 Hz, 1H), 7.94 (d, J = 2.0 Hz, 1H), 7.69 (d, J = 2.0 Hz, 1H), 7.09 (d, J = 2.0 Hz, 1H), 6.94 (d, J = 8.2 Hz, 1H), 6.76 (d, J = 8.2 Hz, 1H), 1.90 (s, 3H), 1.83 (s, 3H). |
| | 3-(2-(benzo[d]thiazol-5-yl)-1H- | | |
| | pyrrolo[2,3-b]pyridin-5-yl)-2,4- | | |
| | dimethylphenol | | |
| 23 | 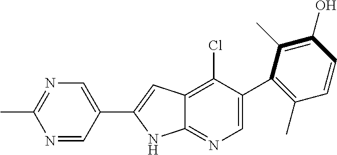 | 406.4 [M + H + CH₃CN]⁺ | (400 MHz, DMSO-d₆) δ 12.75 (s, 1H), 9.31 (s, 2H), 9.22 (s, 1H), 7.99 (s, 1H), 7.26 (d, J = 2.2 Hz, 1H), 6.97 (d, J = 8.6 Hz, 1H), 6.80 (d, J = 8.0 Hz, 1H), 2.68 (s, 3H), 1.83 (s, 3H), 1.76 (s, 3H) |
| | (R)-3-(4-chloro-2-(2- | | |
| | methylpyrimidin-5-yl)-1H- | | |
| | pyrrolo[2,3-b]pyridin-5-yl)-2,4- | | |
| | dimethylphenol | | |
| 24 | 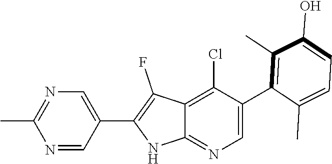 | 383.0 | (400 MHz, DMSO-d₆) δ 12.70 (s, 1H), 9.27 (s, 1H), 9.18 (s, 2H), 8.07 (s, 1H), 6.97 (s, 1H), 6.81 (d, J = 8.2 Hz, 1H), 2.70 (s, 3H), 1.84 (s, 3H), 1.77 (s, 3H) |
| | (S)-3-(4-chloro-3-fluoro-2-(2- | | |
| | methylpyrimidin-5-yl)-1H- | | |
| | pyrrolo[2,3-b]pyridin-5-yl)-2,4- | | |
| | dimethylphenol | | |
| 25 | 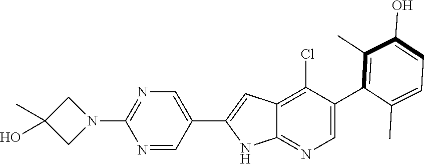 | 436.2 | (400 MHz, DMSO-d₆) δ 12.48 (s, 1H), 9.20 (s, 1H), 8.96 (s, 2H), 7.89 (s, 1H), 6.95 (d, J = 8.8 Hz, 2H), 6.79 (d, J = 8.0 Hz, 1H), 5.68 (s, 1H), 4.00-3.94 (m, 4H), 1.82 (s, 3H), 1.75 (s, 3H), 1.45 (s, 3H) |
| | (S)-1-(5-(4-chloro-5-(3-hydroxy- | | |
| | 2,6-dimethylphenyl)-1H- | | |
| | pyrrolo[2,3-b]pyridin-2- | | |
| | yl)pyrimidin-2-yl)-3- | | |
| | methylazetidin-3-ol | | |
| 26 | 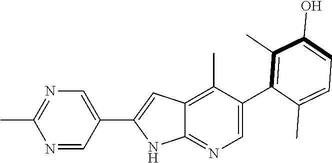 | 345.1 | (400 MHz, DMSO-d₆) δ 12.30 (s, 1H), 9.26 (s, 2H), 9.15 (s, 1H), 7.82 (s, 1H), 7.26 (d, J = 1.6 Hz, 1H), 6.95 (d, J = 8.2 Hz, 1H), 6.77 (d, J = 8.0 Hz, 1H), 2.67 (s, 3H), 2.15 (s, 3H), 1.79 (s, 3H), 1.72 (s, 3H). |
| | (R)-2,4-dimethyl-3-(4-methyl-2-(2- | | |
| | methylpyrimidin-5-yl)-1H- | | |
| | pyrrolo[2,3-b]pyridin-5-yl)phenol | | |
| 27 | 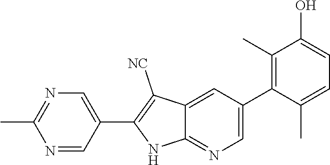 [SMILES: Cc1ncc(-c2[nH]c3ncc(-c4c(C)ccc(O)c4C)cc3c2C#N)cn1] | 356.1 | (400 MHz, CD₃OD) δ 9.30 (s, 2H), 8.18 (s, 1H), 7.88 (d, J = 1.6 Hz, 1H), 6.97 (d, J = 8.4 Hz, 1H), 6.76 (d, J = 8.4 Hz, 1H), 2.82 (s, 3H), 1.93 (s, 3H), 1.89 (s, 3H). |
| | 5-(3-hydroxy-2,6-dimethylphenyl)- | | |
| | 2-(2-methylpyrimidin-5-yl)-1H- | | |
| | pyrrolo[2,3-b]pyridine-3- | | |
| | carbonitrile | | |

### Example 29: Preparation of 2,4-dimethyl-3-(2-(5-methylpyrazin-2-yl)-1H-pyrrolo[2,3-b]pyridin-5-yl)phenol

The example 29 was synthesized from compound 29-1, which was prepared according to the methods described in example 12 with analogous starting material.

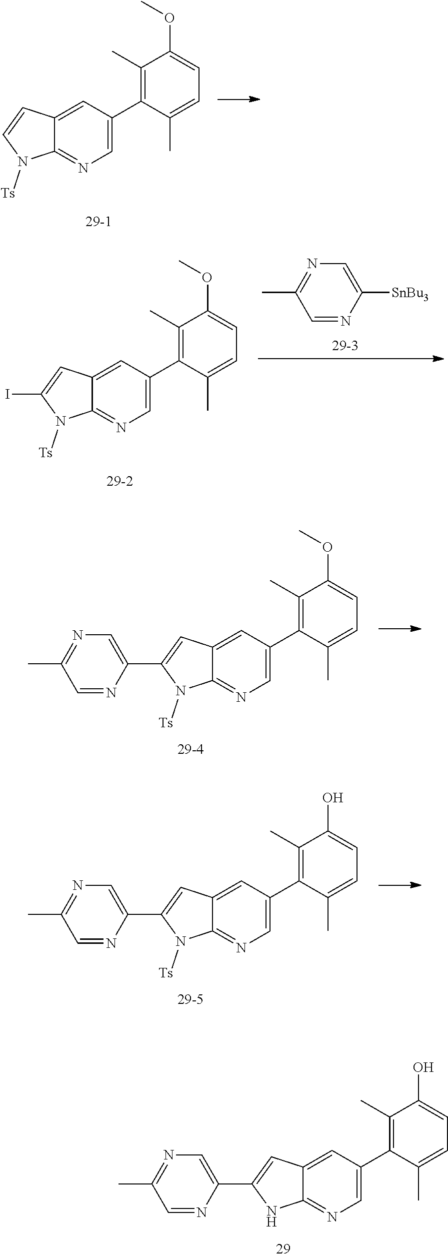

### Step 1: Preparation of Compound 29-2

To a solution of compound 29-1 (585 mg, 1.439 mmol) in THF (10 mL) was added LDA (1.1 mL, 2.159 mmol) at −78° C. The mixture was stirred at this temperature for 0.5 h. Then I₂ (766.05 mg, 5.361 mmol) was added and stirred at −78° C. for 1 h. The mixture was quenched with NH₄Cl (sat.aq, 20 mL) and extracted with EA (100 mL). The organic layer was washed with Na₂SO₃ (sat.aq, 30 mL). The organic layer was dried over Na₂SO₄ and concentrated to dryness. The residue was purified by silica gel column chromatography to afford compound 29-2 (550 mg, 1.03 mmol, 71.78%). LCMS: 533.4 [M+H]⁺.

### Step 2: Preparation of Compound 29-4

To a solution of compound 29-2 (136 mg, 0.255 mmol) in DMF (2 mL) was added compound 29-3 (97.88 mg, 0.281 mmol), Pd(PPh₃)₄ (29.47 mg, 0.026 mmol), CuI (9.71 mg, 0.051 mmol) and LiCl (32.43 mg, 0.765 mmol). The mixture was stirred at 120° C. for 2 h. The reaction mixture was filtered. The filtrate was concentrated to dryness. The residue was purified by prep-TLC to afford compound 29-4. LCMS: 499.7 [M+H]⁺.

### Step 3: Preparation of Compound 29-5

To a solution of compound 29-4 (48 mg, 0.096 mmol) in DCM (5 mL) was added BBr₃ (0.241 mL, 2.0 M in DCM). The mixture was stirred at room temperature under N₂ for 1 h. The reaction was quenched with MeOH (5 mL). The mixture was concentrated to dryness. The residue was purified by prep-TLC to afford compound 29-5. LCMS: 485.2 [M+H]⁺.

### Step 4: Preparation of Example 29

To a solution of compound 29-5 (28 mg, 0.058 mmol) in EtOH (3 mL) was added NaOH (0.6 mL, 1.0 M). The mixture was stirred at 60° C. for 2 h. The mixture was concentrated to dryness. The residue was purified by prep-HPLC to afford example 29. LCMS: 331.0 [M+H]⁺; ¹H NMR: (400 MHz, CD₃OD) δ 9.10 (s, 1H), 8.61 (s, 1H), 8.03 (s, 1H), 7.89 (s, 1H), 7.29 (s, 1H), 6.95 (s, 1H), 6.75 (s, 1H), 2.61 (s, 3H), 1.94 (s, 3H), 1.90 (s, 3H).

### Example 30: Preparation of 3-(4-amino-2-(2-methylpyrimidin-5-yl)-1H-pyrrolo[2,3-b]pyridin-5-yl)-2,4-dimethylphenol

The example 30 was synthesized from compound 30-1, which was prepared according to the methods described in example 12 with analogous starting material.

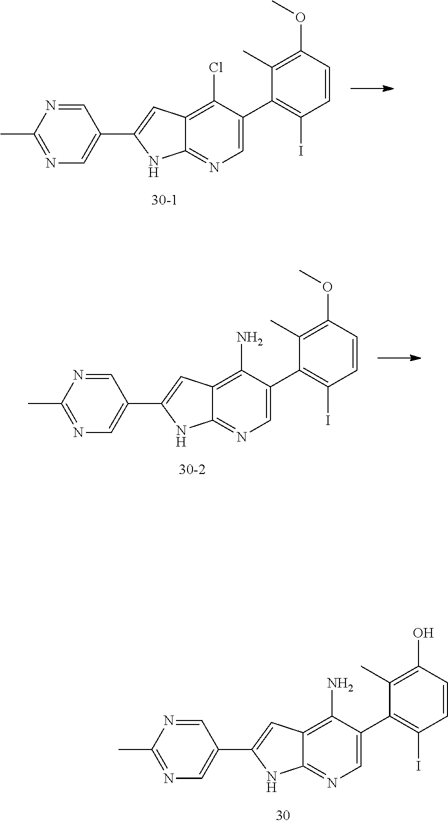 [SMILES: COc1ccc(I)c(-c2cnc3[nH]c(-c4cnc(C)nc4)cc3c2Cl)c1C ; COc1ccc(I)c(-c2cnc3[nH]c(-c4cnc(C)nc4)cc3c2N)c1C ; Cc1ncc(-c2cc3c(N)c(-c4c(I)ccc(O)c4C)cnc3[nH]2)cn1]

### Step 1: Preparation of Compound 30-2

To a solution of compound 30-1 (300.0 mg, 0.563 mmol) in DMSO (6.0 mL) were added CuI (750.6 mg, 3.940 mmol), L-proline (453.6 mg, 3.940 mmol) and NaN₃ (512.2 mg, 7.880 mmol). The reaction mixture was stirred at 140 [symbol] under N₂ for 18 h. The reaction mixture was diluted with water (20 mL) and extracted with EA (20 mL×3). The combined organic layer was washed with brine (20 mL), dried over Na₂SO₄, filtered and concentrated. The residue was purified by prep-TLC to afford compound 30-2. LCMS: 360.2 [M+H]⁺.

### Step 2: Preparation of Example 30

To a solution of compound 30-2 (40 mg, 0.111 mmol) in DCM (1.5 mL) was added BBr₃ (0.1 mL, 0.111 mmol) The mixture was stirred at rt for 2 h. The reaction mixture was carefully added into MeOH (5 mL) while stirring, and the pH was adjusted to 7-8 with NH₃/MeOH. After filtration, the filtrate was concentrated to dryness. The residue was purified by silica gel column chromatography to afford example 30. LCMS: 346.2 [M+H]⁺; ¹H NMR: (400 MHz, CD₃OD) S 9.12 (s, 2H), 7.46-7.21 (m, 3H), 7.01 (d, J=8.0 Hz, 1H), 6.77 (d, J=8.0 Hz, 1H), 2.74 (s, 3H), 1.96 (s, 3H), 1.92 (s, 3H).

### Example 31: Preparation of (R)-2,4-dimethyl-3-(2-(2-methylpyrimidin-5-yl)-4-(trifluoromethyl)-1H-pyrrolo[2,3-b]pyridin-5-yl)phenol

The example 31 was prepared according to the following synthetic procedures.

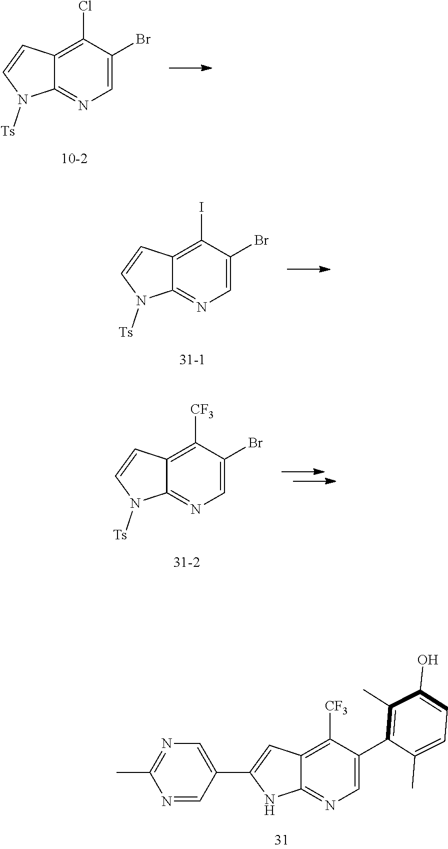

### Step 1: Preparation of Compound 31-1

To a solution of compound 10-2 (4 g, 10.372 mmol) in CH₃CN (60 mL) were added NaI (7.77 g, 51.859 mmol) and acetyl chloride (2.212 mL, 31.115 mmol). The mixture was stirred at 80° C. under N₂ overnight. The reaction mixture was diluted with water (50 mL) and extracted with EA (100 mL×3). The combined organic layer was washed with brine (300 mL), dried over Na₂SO₄, filtered, and concentrated to dryness. The residue was purified by silica gel column chromatography to afford compound 31-1 (3.2 g, 6.707 mmol, 64.67%). LCMS: 476.8 [M+H]⁺.

### Step 2: Preparation of Compound 31-2

To a solution of compound 31-1 (600 mg, 1.258 mmol) in DMF (20 mL) were added CuI (239.50 mg, 1.258 mmol) and methyl 2,2-difluoro-2-fluoro-sulfonyl acetate (965.82 mg, 5.030 mmol) at 80° C. The mixture was stirred at this temperature for 12 h. The mixture was quenched with H₂O (20 mL) and extracted with EA (100 mL). The organic layer was dried over Na₂SO₄ and concentrated to dryness. The residue was purified by silica gel column chromatography to afford compound 31-2 (450 mg, 1.073 mmol, 85.36%). LCMS: 418.9 [M+H]⁺.

### Step 3: Preparation of Example 31

The example 31 was prepared from compound 31-2 according to the methods described in example 12. Spectrum data of example 31: LCMS: 399.1 [M+H]⁺; ¹H NMR: (400 MHz, DMSO-d₆) δ 12.97 (s, 1H), 9.35 (s, 2H), 9.22 (s, 1H), 8.03 (s, 1H), 7.34 (s, 1H), 6.94 (d, J=8.0 Hz, 1H), 6.78 (d, J=8.0 Hz, 1H), 2.69 (s, 3H), 1.78 (s, 3H), 1.71 (s, 3H).

### Example 32: Preparation of (S)-5-(3-hydroxy-2,6-dimethylphenyl)-2-(2-methylpyrimidin-5-yl)-1H-pyrrolo[2,3-b]pyridine-4-carbonitrile

The example 32 was prepared according to the following synthetic procedures.

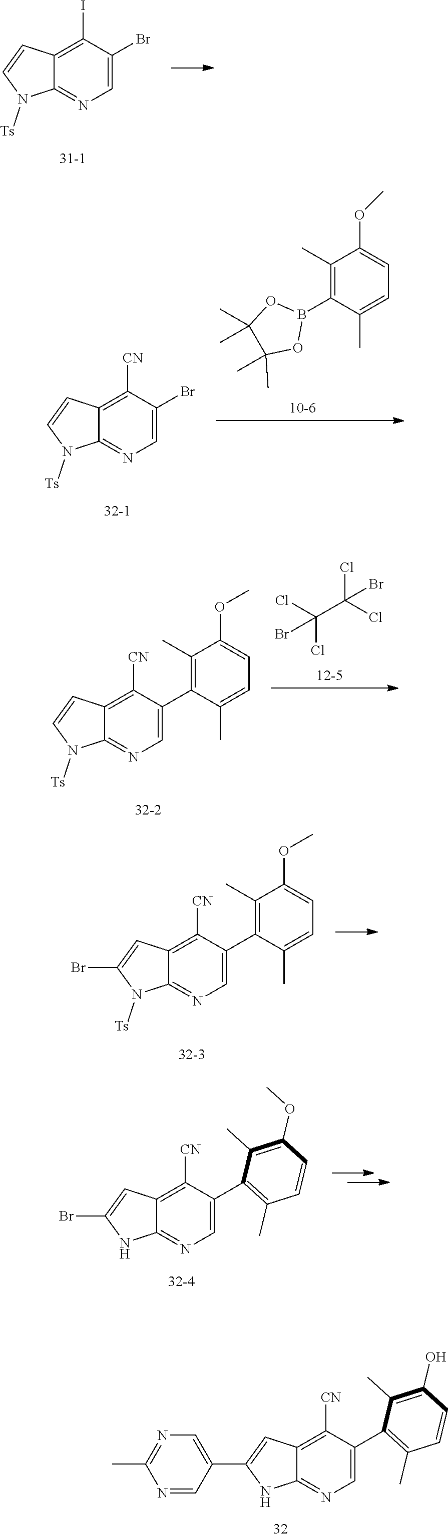

### Step 1: Preparation of Compound 32-1

To a solution of compound 31-1 (23.5 g, 49.255 mmol) in DMF (235 mL) was added Zn(CN)₂ (2.891 g, 24.627 mmol) and Pd(PPh₃)₄ (5.692 g, 4.925 mmol). The mixture was stirred at 110° C. for 16 h. The mixture was quenched by water (100 mL), which was extracted with EtOAc (500 mL×3). The organic layer was washed with brine, concentrated, and purified by silica gel column chromatography to afford compound 32-1 (25 g, 35.983 mmol, 73.1%). LCMS: 378.1 [M+H]⁺.

### Step 2: Preparation of Compound 32-2

To a solution of compound 32-1 (10 g, 26.582 mmol) and compound 10-6 (10.5 g, 39.872 mmol) in dioxane (100 mL) and H₂O (20 mL) was added Pd(dtbpf)Cl₂ (1.7 g, 2.658 mmol) and K₂CO₃ (11.0 g, 79.745 mmol). The mixture was stirred at 90° C. under N₂ for 16 h. The mixture was concentrated. The residue was purified by column chromatography on silica gel to compound 32-2 (8.32 g, 19.299 mmol, 72.6%). LCMS: 432.3 [M+H]⁺.

### Step 3: Preparation of Compound 32-3

To a solution of compound 32-2 (27 g, 62.571 mmol) in THF (500 mL) was added LDA (62.57 mL, 125.142 mmol) in drops at −78 [symbol] . The mixture was stirred at −78 [symbol] for 30 min. Then compound 12-5 (40.7 g, 125.142 mmol) in THF (5 mL) was added. The mixture was stirred at −78 [symbol] for 1 h and then warmed to rt for 16 h. The mixture was quenched by st.aq NH₄Cl (100 mL), extracted with EA (50 mL×3). The combined organic layer was washed with brine, dried over Na₂SO₄, filtered and concentrated. The residue was purified by column chromatography on silica gel to afford compound 32-3 (18 g, 35.266 mmol, 56.4%). LCMS: 511.8 [M+H]⁺.

### Step 4: Preparation of Compound 32-4

A mixture of compound 32-3 (7 g, 13.715 mmol) and NaOH (1 M, 137.15 mL, 137.147 mmol) in EtOH (150 mL) and MeOH (150 mL) was heated to 60 [symbol] for 2 h. The mixture was cooled to 0 [symbol] , adjusted to pH=6 by iN HCl, extracted with EA (200 mL×3). The combined organic layer was washed with brine, dried over Na₂SO₄, filtered and concentrated. The residue was purified by column chromatography on silica gel and further purified by SFC to afford compound 32-4. LCMS: 357.9 [M+H]⁺. ¹H NMR: (400 MHz, DMSO-d₆) δ 13.32 (s, 1H), 8.30-8.02 (m, 1H), 7.18 (d, J=7.6 Hz, 1H), 7.06-6.92 (m, 1H), 6.85 (s, 1H), 3.83 (s, 3H), 1.88 (s, 3H), 1.80 (s, 3H).

### Step 5: Preparation of Example 32

The example 32 was prepared from compound 32-4 according to the methods described in example 12. Spectrum data of example 32: LCMS: 356.2 [M+H]⁺; ¹H NMR: (400 MHz, DMSO-d₆) δ 13.09 (s, 1H), 9.37 (s, 3H), 8.21 (s, 1H), 7.43 (s, 1H), 7.01 (d, J=8.2 Hz, 1H), 6.85 (d, J=8.2 Hz, 1H), 2.70 (s, 3H), 1.87 (s, 3H), 1.79 (s, 3H).

### Example 33: Preparation of (S)-3-chloro-5-(3-hydroxy-2,6-dimethylphenyl)-2-(2-methylpyrimidin-5-yl)-1H-pyrrolo[2,3-b]pyridine-4-carbonitrile

The title compound was synthesized from compound 33-1, which was prepared according to the methods described in example 32 with analogous starting materials.

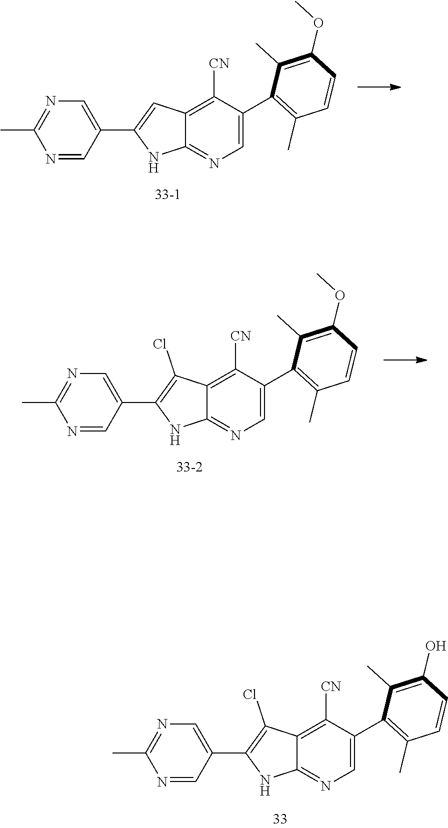

### Step 1: Preparation of Compound 33-2

To a solution of compound 33-1 (40 mg, 0.108 mmol) in CH₃CN (2 mL) was added NCS (14.4 mg, 0.108 mmol). The mixture was stirred at 40° C. for 16 h. The mixture was diluted with H₂O (15 mL) and extracted with DCM (10 mL×3). Combined organic layers were washed with brine (10 mL×3), dried over Na₂SO₄, filtered and concentrated. The residue was purified by silica gel column chromatography to afford compound 33-2. LCMS: 404.1 [M+H]⁺.

### Step 2: Preparation of Example 33

To a solution of compound 33-2 (30 mg, 0.0743 mmol) in DCM (3 mL) was added BBr₃ (186.2 mg, 0.743 mmol). The mixture was stirred at room temperature under N₂ for 1 h. The reaction was quenched with MeOH (5 mL). The mixture was concentrated to dryness. The residue was purified by prep-HPLC to afford example 33. LCMS: 390.1 [M+H]⁺; ¹H NMR: (400 MHz, DMSO-d₆) δ 13.42 (s, 1H), 9.40 (s, 1H), 9.23 (s, 2H), 8.33 (s, 1H), 7.02 (d, J=8.4 Hz, 1H), 6.86 (d, J=8.4 Hz, 1H), 2.74 (s, 3H), 1.87 (s, 3H), 1.79 (s, 3H).

### Example 34: Preparation of 5-(3-hydroxy-2,6-dimethylphenyl)-2-(2-methylpyrimidin-5-yl)-1H-pyrrolo[2,3-b]pyridine-4-carboxamide

The title compound was synthesized from compound 33-1, which was prepared according to the methods described in example 32 with analogous starting materials.

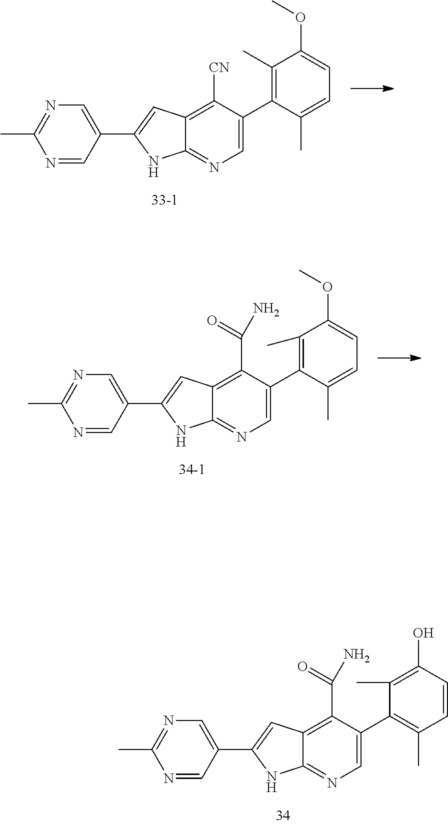 [SMILES: COc1ccc(C)c(-c2cnc3[nH]c(-c4cnc(C)nc4)cc3c2C#N)c1C ; COc1ccc(C)c(-c2cnc3[nH]c(-c4cnc(C)nc4)cc3c2C(N)=O)c1C ; Cc1ncc(-c2cc3c(C(N)=O)c(-c4c(C)ccc(O)c4C)cnc3[nH]2)cn1]

### Step 1: Preparation of Compound 34-1

To a solution of compound 33-1 (50 mg, 0.135 mmol) in MeOH (1 mL) and H₂O (0.5 mL) was added H₂O₂ (85.72 mg, 0.677 mmol) and NaOH (16.24 mg, 0.406 mmol). The mixture was stirred at 40° C. for 16 h. The mixture was filtered and concentrated to dryness. The residue was purified by silica gel column chromatography to afford compound 34-1. LCMS: 388.4 [M+H]⁺.

### Step 2: Preparation of Example 34

To a solution of compound 34-1 (22 mg, 0.057 mmol) in DCM (5 mL) was added BBr₃ (0.142 mL, 0.284 mmol). The mixture was stirred at room temperature under N₂ for 0.5 h. The reaction was quenched with MeOH (5 mL). The mixture was concentrated to dryness. The residue was purified by prep-HPLC to afford example 34. LCMS: 374.3 [M+H]⁺; ¹H NMR: (400 MHz, DMSO-d₆) δ 12.51 (s, 1H), 9.29 (s, 2H), 9.05 (s, 1H), 7.90 (s, 1H), 7.48 (s, 11H), 7.42 (s, 1H), 7.16 (s, 11H), 6.86 (d, J=8.0 Hz, 1H), 6.71 (d, J=8.0 Hz, 1H), 2.68 (s, 3H), 1.87 (s, 3H), 1.79 (s, 3H).

The following examples 35-81 were prepared according to the methods described in examples 32-34 with analogous starting materials.

| | | LCMS | |
|---|---|---|---|
| | | [M + H]+ | |
| | | (except | |
| Ex- | | where | |
| ample | Structure | indicated) | ¹H NMR |
| 35 | 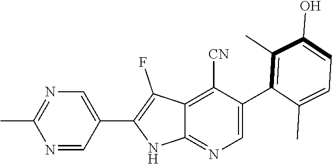 | 415.1 [M + H + CH₃CN]⁺ | (400 MHz, DMSO-d₆) δ 13.05 (s, 1H), 9.38 (s, 1H), 9.23 (s, 2H), 8.30 (s, 1H), 7.02 (d, J = 8.2 Hz, 1H), 6.86 (d, J = 8.2 Hz, 1H), 2.72 (s, 3H), 1.88 (s, 3H), 1.80 (s, 3H). |
| | (S)-3-fluoro-5-(3-hydroxy-2,6- | | |
| | dimethylphenyl)-2-(2- | | |
| | methylpyrimidin-5-yl)-1H- | | |
| | pyrrolo[2,3-b]pyridine-4- | | |
| | carbonitrile | | |
| 36 | 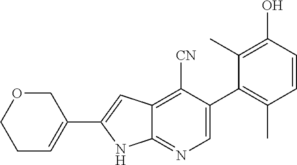 [SMILES: Cc1ccc(O)c(C)c1-c1cnc2[nH]c(C3=CCCOC3)cc2c1C#N] | 346.1 | (400 MHz, CD₃OD) δ 8.04 (s, 1H), 6.99 (d, J = 8.4 Hz, 1H), 6.80 (d, J = 8.4 Hz, 1H), 6.71 (d, J = 4.4 Hz, 1H), 6.54 (s, 1H), 4.59 (d, J = 2.0 Hz, 2H), 3.89 (t, J = 5.6 Hz, 2H), 2.43 (d, J = 4.4 Hz, 2H), 1.91 (s, 3H), 1.86 (s, 3H). |
| | 2-(5,6-dihydro-2H-pyran-3-yl)-5- | | |
| | (3-hydroxy-2,6-dimethylphenyl)- | | |
| | 1H-pyrrolo[2,3-b]pyridine-4- | | |
| | carbonitrile | | |
| 37 | 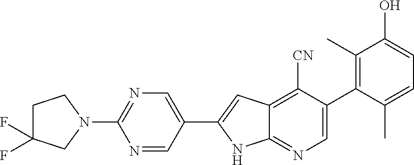 [SMILES: Cc1ccc(O)c(C)c1-c1cnc2[nH]c(-c3cnc(N4CCC(F)(F)C4)nc3)cc2c1C#N] | 447.2 | (400 MHz, DMSO-d₆) δ 12.84 (s, 1H), 9.35 (s, 1H), 9.10 (s, 2H), 8.10 (s, 1H), 7.17 (s, 1H), 7.01 (d, J = 8.4 Hz, 1H), 6.84 (d, J = 8.4 Hz, 1H), 4.00 (t, J = 13.2 Hz, 2H), 3.82 (t, J = 7.2 Hz, 2H), 2.64-2.55 (m, 2H), 1.87 (s, 3H), 1.79 (s, 3H). |
| | 2-(2-(3,3-difluoropyrrolidin-1- | | |
| | yl)pyrimidin-5-yl)-5-(3-hydroxy- | | |
| | 2,6-dimethylphenyl)-1H- | | |
| | pyrrolo[2,3-b]pyridine-4- | | |
| | carbonitrile | | |
| 38 | 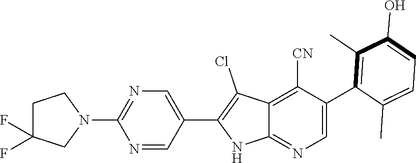 | 481.0 | (400 MHz, DMSO-d₆) δ 13.18 (s, 1H), 9.37 (s, 1H), 8.96 (s, 1H), 8.23 (s, 1H), 7.02 (d, J = 8.0 Hz, 1H), 6.85 (d, J = 8.0 Hz, 1H), 4.02 (t, J = 9.0 Hz, 2H), 3.84 (dd, J = 9.0, 5.8 Hz, 2H), 2.62-2.56 (m, 2H), 1.87 (s, 3H), 1.79 (s, 3H). |
| | (R)-3-chloro-2-(2-(3,3- | | |
| | difluoropyrrolidin-1- | | |
| | yl)pyrimidin-5-yl)-5-(3-hydroxy- | | |
| | 2,6-dimethylphenyl)-1H- | | |
| | pyrrolo[2,3-b]pyridine-4- | | |
| | carbonitrile | | |
| 39 | 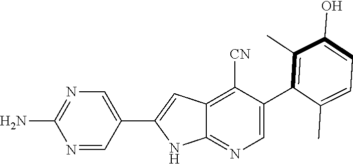 | 357.1 | (400 MHz, DMSO-d₆) δ 12.72 (s, 1H), 9.37 (s, 1H), 8.93 (s, 2H), 8.06 (s, 1H), 7.12 (s, 2H), 7.08 (s, 1H), 7.00 (d, J = 8.4 Hz, 1H), 6.84 (d, J = 8.4 Hz, 1H), 1.87 (s, 3H), 1.79 (s, 3H). |
| | (R)-2-(2-aminopyrimidin-5-yl)-5- | | |
| | (3-hydroxy-2,6-dimethylphenyl)- | | |
| | 1H-pyrrolo[2,3-b]pyridine-4- | | |
| | carbonitrile | | |
| 40 | 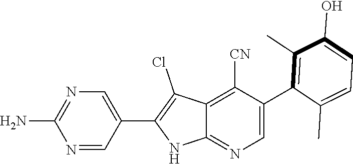 | 391.4 | (400 MHz, DMSO-d₆) δ 13.05 (s, 1H), 9.36 (s, 1H), 8.80 (s, 2H), 8.20 (s, 1H), 7.30 (s, 2H), 7.01 (d, J = 8.4 Hz, 1H), 6.85 (d, J = 8.4 Hz, 1H), 1.86 (s, 3H), 1.79 (s, 3H). |
| | (R)-2-(2-aminopyrimidin-5-yl)-3- | | |
| | chloro-5-(3-hydroxy-2,6- | | |
| | dimethylphenyl)-1H-pyrrolo[2,3- | | |
| | b]pyridine-4-carbonitrile | | |
| 41 | 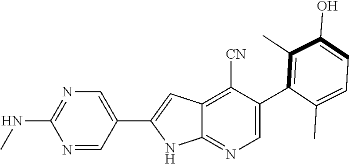 | 412.2 [M + H + CH₃CN]⁺ | (400 MHz, DMSO-d₆) δ 12.74 (s, 1H), 9.32 (s, 1H), 8.97 (s, 2H), 8.06 (s, 1H), 7.59 (d, J = 4.8 Hz, 1H), 7.08 (s, 1H), 7.00 (d, J = 8.4 Hz, 1H), 6.84 (d, J = 8.4 Hz, 1H), 2.89 (d, J = 4.0 Hz, 3H), 1.87 (s, 3H), 1.79 (s, 3H). |
| | (R)-5-(3-hydroxy-2,6- | | |
| | dimethylphenyl)-2-(2- | | |
| | (methylamino)pyrimidin-5-y1)- | | |
| | 1H-pyrrolo[2,3-b]pyridine-4- | | |
| | carbonitrile | | |
| 42 | 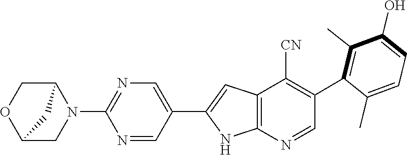 | 439.2 | (400 MHz, DMSO-d₆) δ 12.77 (s, 1H), 9.33 (s, 1H), 9.03 (s, 2H), 8.07 (s, 1H), 7.11 (s, 1H), 7.00 (d, J = 8.4 Hz, 1H), 6.84 (d, J = 8.4 Hz, 1H), 5.05 (s, 1H), 4.72 (s, 1H), 3.84 (d, J = 6.4 Hz, 1H), 3.72 (d, J = 7.6 Hz, 1H), 3.56 (d, J = 10.0 Hz, 1H), 3.47 (d, J = 11.2 Hz, 1H), 1.98 (d, J = 9.6 Hz, 1H), 1.91 (d, J = 10.4 Hz, 1H), 1.87 (s, 3H), 1.79 |
| | (R)-2-(2-((1S,4S)-2-oxa-5- | | (s, 3H). |
| | azabicyclo[2.2.1]heptan-5- | | |
| | yl)pyrimidin-5-yl)-5-(3-hydroxy- | | |
| | 2,6-dimethylphenyl)-1H- | | |
| | pyrrolo[2,3-b]pyridine-4- | | |
| | carbonitrile | | |
| 43 | 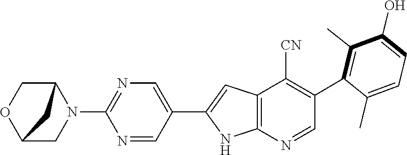 | 439.2 | (400 MHz, DMSO-d₆) δ 12.80 (s, 1H), 9.34 (s, 1H), 9.03 (s, 2H), 8.08 (s, 1H), 7.11 (s, 1H), 7.00 (d, J = 8.4 Hz, 1H), 6.84 (d, J = 8.4 Hz, 1H), 5.05 (s, 1H), 4.72 (s, 1H), 3.84 (d, J = 6.4 Hz, 1H), 3.72 (d, J = 7.2 Hz, 1H), 3.56 (d, J = 10.0 Hz, 1H), 3.46 (s, 1H), 1.97 (d, J = 7.6 Hz, 1H), 1.92 (s, 1H), 1.87 (s, 3H), 1.79 (s, 3H). |
| | (R)-2-(2-((1R,4R)-2-oxa-5- | | |
| | azabicyclo[2.2.1]heptan-5- | | |
| | yl)pyrimidin-5-yl)-5-(3-hydroxy- | | |
| | 2,6-dimethylphenyl)-1H- | | |
| | pyrrolo[2,3-b]pyridine-4- | | |
| | carbonitrile | | |
| 44 | 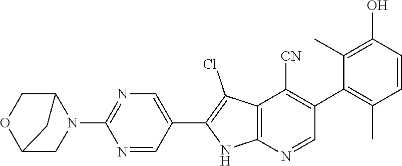 [SMILES: Cc1ccc(O)c(C)c1-c1cnc2[nH]c(-c3cnc(N4CC5CC4CO5)nc3)c(Cl)c2c1C#N] | 473.1 | (400 MHz, DMSO-d₆) δ 9.41 (s, 1H), 8.93 (d, J = 2.4 Hz, 2H), 8.45 (s, 1H), 8.15 (s, 1H), 7.01 (d, J = 8.0 Hz, 1H), 6.84 (d, J = 8.0 Hz, 1H), 5.07 (s, 1H), 4.73 (s, 1H), 3.85 (d, J = 7.2 Hz, 1H), 3.74 (d, J = 7.2 Hz, 1H), 3.58 (d, J = 11.2 Hz, 2H), 2.00-1.91 (m, 2H), 1.87 (s, 3H), 1.79 (s, 3H). |
| | 2-(2-(2-oxa-5- | | |
| | azabicyclo[2.2.1]heptan-5- | | |
| | yl)pyrimidin-5-yl)-3-chloro-5-(3- | | |
| | hydroxy-2,6-dimethylphenyl)- | | |
| | 1H-pyrrolo[2,3-b]pyridine-4- | | |
| | carbonitrile | | |
| 45 | 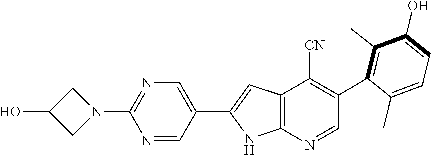 | 413.4 | (400 MHz, CD₃OD) δ 8.89 (s, 2H), 8.04 (s, 1H), 7.04-6.97 (m, 2H), 6.81 (d, J = 8.4 Hz, 1H), 4.74-4.71 (m, 1H), 4.56 (s, 2H), 4.44 (dd, J = 10.8, 6.4Hz, 2H), 4.00 (dd, J = 10.8, 4.4 Hz, 2H), 1.93 (s, 3H), 1.88 (s, 3H). |
| | (R)-5-(3-hydroxy-2,6- | | |
| | dimethylphenyl)-2-(2-(3- | | |
| | hydroxyazetidin-1-yl)pyrimidin- | | |
| | 5-yl)-1H-pyrrolo[2,3-b]pyridine- | | |
| | 4-carbonitrile | | |
| 46 | 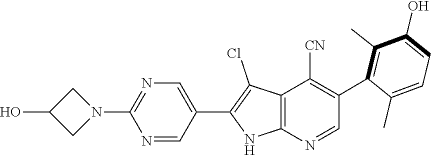 | 447.5 | (400 MHz, DMSO-d₆) δ 13.12 (s, 1H), 9.35 (s, 1H), 8.88 (s, 2H), 8.21 (s, 1H), 7.01 (d, J = 8.4 Hz, 1H), 6.85 (d, J = 8.0 Hz, 1H), 4.64-4.61 (m, 1H), 4.43-4.27 (m, 2H), 3.89 (dd, J = 10.0, 4.4 Hz, 2H), 1.86 (s, 3H), 1.79 (s, 3H). |
| | (R)-3-chloro-5-(3-hydroxy-2,6- | | |
| | dimethylphenyl)-2-(2-(3- | | |
| | hydroxyazetidin-1-yl)pyrimidin- | | |
| | 5-y1)-1H-pyrrolo[2,3-b]pyridine- | | |
| | 4-carbonitrile | | |
| 47 | 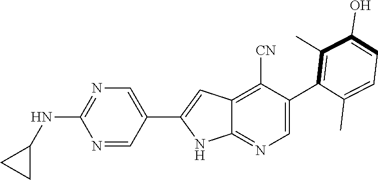 | 397.5 | (400 MHz, DMSO-d₆) δ 12.76 (s, 1H), 9.33 (s, 1H), 8.99 (s, 2H), 8.07 (s, 1H), 7.88 (d, J = 3.2 Hz, 1H), 7.09 (d, J = 1.6 Hz, 1H), 7.00 (d, J = 8.4 Hz, 1H), 6.84 (d, J = 8.4 Hz, 1H), 2.82-2.79 (m, 1H), 1.87 (s, 3H), 1.79 (s, 3H), 0.79- 0.67 (m, 2H), 0.59-0.47 (m, 2H). |
| | (R)-2-(2- | | |
| | (cyclopropylamino)pyrimidin-5- | | |
| | yl)-5-(3-hydroxy-2,6- | | |
| | dimethylphenyl)-1H-pyrrolo[2,3- | | |
| | b]pyridine-4-carbonitrile | | |
| 48 | 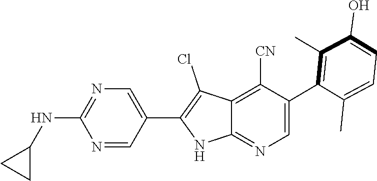 | 431.5 | (400 MHz, DMSO-d₆) δ 13.08 (s, 1H), 9.37 (s, 1H), 8.86 (s, 2H), 8.21 (d, J = 1.2 Hz, 1H), 8.00 (s, 1H), 7.02 (d, J = 8.0 Hz, 1H), 6.85 (d, J = 8.4 Hz, 1H), 2.82 (s, 1H), 1.87 (s, 3H), 1.79 (s, 3H), 0.78- 0.72 (m, 2H), 0.59-0.53 (m, 2H). |
| | (R)-3-chloro-2-(2- | | |
| | (cyclopropylamino)pyrimidin-5- | | |
| | yl)-5-(3-hydroxy-2,6- | | |
| | dimethylphenyl)-1H-pyrrolo[2,3- | | |
| | b]pyridine-4-carbonitrile | | |
| 49 | 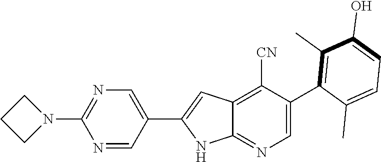 | 397.4 | (400 MHz, MeOD-d₄) δ 8.88 (s, 2H), 8.04 (s, 1H), 7.01 (d, J = 8.4 Hz, 1H), 6.98 (s, 1H), 6.82 (d, J = 8.4 Hz, 1H), 4.29-4.19 (m, 4H), 2.49-2.43 (m, 1H), 1.93 (s, 3H), 1.88 (s, 3H), 1.45-1.42 (m, 2H). |
| | (R)-2-(2-(azetidin-1- | | |
| | yl)pyrimidin-5-y1)-5-(3-hydroxy- | | |
| | 2,6-dimethylphenyl)-1H- | | |
| | pyrrolo[2,3-b]pyridine-4- | | |
| | carbonitrile | | |
| 50 | 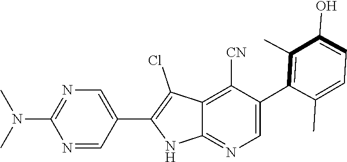 | 419.1 | (400 MHz, DMSO-d₆) δ 13.10 (s, 1H), 9.35 (s, 1H), 8.90 (s, 2H), 8.20 (s, 1H), 7.01 (d, J = 8.4 Hz, 1H), 6.85 (d, J = 8.4 Hz, 1H), 3.23 (s, 6H), 1.87 (s, 3H), 1.79 (s, 3H). |
| | (S)-3-chloro-2-(2- | | |
| | (dimethylamino)pyrimidin-5-yl)- | | |
| | 5-(3-hydroxy-2,6- | | |
| | dimethylphenyl)-1H-pyrrolo[2,3- | | |
| | b]pyridine-4-carbonitrile | | |
| 51 | 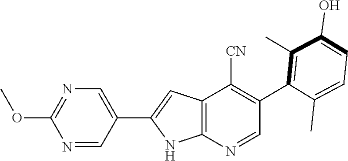 | 413.2 [M + H + CH₃CN]⁺ | (400 MHz, DMSO-d₆) δ 13.01 (s, 1H), 9.36 (s, 1H), 9.28 (s, 2H), 8.17 (s, 1H), 7.32 (s, 1H), 7.01 (d, J = 8.4 Hz, 1H), 6.84 (d, J = 8.4 Hz, 1H), 4.01 (s, 3H), 1.87 (s, 3H), 1.79 (s, 3H). |
| | (R)-5-(3-hydroxy-2,6- | | |
| | dimethylphenyl)-2-(2- | | |
| | methoxypyrimidin-5-yl)-1H- | | |
| | pyrrolo[2,3-b]pyridine-4- | | |
| | carbonitrile | | |
| 52 | 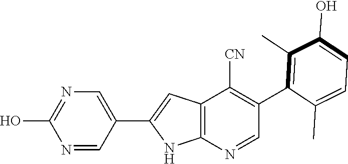 | 358.1 | (400 MHz, DMSO-d₆) δ 12.70 (s, 1H), 9.33 (s, 1H), 8.97 (s, 2H), 8.10 (s, 1H), 7.17 (d, J = 1.8 Hz, 1H), 7.00 (d, J = 8.2 Hz, 1H), 6.84 (d, J = 8.2 Hz, 1H), 1.86 (s, 3H), 1.79 (s, 3H). |
| | (R)-5-(3-hydroxy-2,6- | | |
| | dimethylphenyl)-2-(2- | | |
| | hydroxypyrimidin-5-yl)-1H- | | |
| | pyrrolo[2,3-b]pyridine-4- | | |
| | carbonitrile | | |
| 53 | 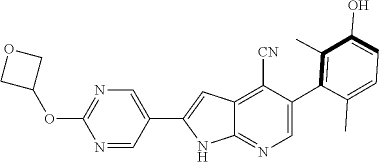 | 414.2 | (400 MHz, DMSO-d₆) δ 13.00 (s, 1H), 9.36 (s, 1H), 9.28 (s, 2H), 8.18 (s, 1H), 7.34 (s, 1H), 7.01 (d, J = 8.2 Hz, 1H), 6.85 (d, J = 8.2 Hz, 1H), 5.66 (t, J = 5.6 Hz, 1H), 4.94 (t, J = 7.2 Hz, 2H), 4.65 (dd, J = 8.0, 5.2 Hz, 2H), 1.87 (s, 3H), 1.79 (s, 3H). |
| | (S)-5-(3-hydroxy-2,6- | | |
| | dimethylphenyl)-2-(2-(oxetan-3- | | |
| | yloxy)pyrimidin-5-yl)-1H- | | |
| | pyrrolo[2,3-b]pyridine-4- | | |
| | carbonitrile | | |
| 54 | 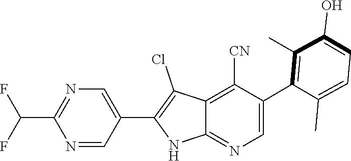 | 426.5 | (400 MHz, DMSO-d₆) δ 13.57 (s, 1H), 9.50 (d, J = 10.0 Hz, 2H), 9.41 (s, 1H), 8.38 (s, 1H), 7.28- 6.95 (m, 2H), 6.87 (d, J = 8.2 Hz, 1H), 1.88 (s, 3H), 1.80 (s, 3H). |
| | (S)-3-chloro-2-(2- | | |
| | (difluoromethyl)pyrimidin-5-yl)- | | |
| | 5-(3-hydroxy-2,6- | | |
| | dimethylphenyl)-1H-pyrrolo[2,3- | | |
| | b]pyridine-4-carbonitrile | | |
| 55 | 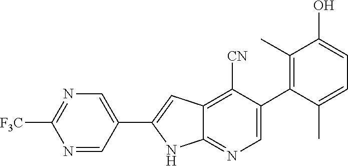 [SMILES: Cc1ccc(O)c(C)c1-c1cnc2[nH]c(-c3cnc(C(F)(F)F)nc3)cc2c1C#N] | 410.4 | (400 MHz, DMSO-d₆) δ 13.32 (s, 1H), 9.72 (s, 2H), 9.42 (s, 1H), 8.31 (s, 1H), 7.68 (s, 1H), 7.03 (d, J = 8.2 Hz, 1H), 6.86 (d, J = 8.2 Hz, 1H), 1.88 (s, 3H), 1.81 (s, 3H). |
| | 5-(3-hydroxy-2,6- | | |
| | dimethylphenyl)-2-(2- | | |
| | (trifluoromethyl)pyrimidin-5-yl)- | | |
| | 1H-pyrrolo[2,3-b]pyridine-4- | | |
| | carbonitrile | | |
| 56 | 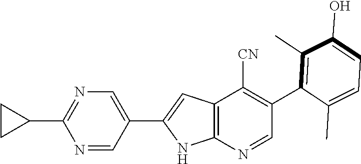 | 382.1 | (400 MHz, DMSO-d₆) δ 13.05 (s, 1H), 9.36 (s, 1H), 9.29 (s, 2H), 8.19 (s, 1H), 7.37 (s, 1H), 7.01 (d, J = 8.4 Hz, 1H), 6.85 (d, J = 8.4 Hz, 1H), 2.32-2.25 (m, 1H), 1.87 (s, 3H), 1.79 (s, 3H), 1.13-1.09 (m, 4H). |
| | (S)-2-(2-cyclopropylpyrimidin-5- | | |
| | yl)-5-(3-hydroxy-2,6- | | |
| | dimethylphenyl)-1H-pyrrolo[2,3- | | |
| | b]pyridine-4-carbonitrile | | |
| 57 | 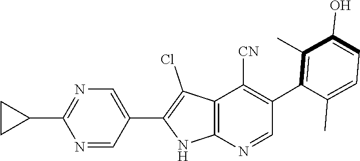 | 416.0 | (400 MHz, DMSO-d₆) δ 13.33 (s, 1H), 9.37 (s, 1H), 9.16 (s, 2H), 8.29 (s, 1H), 7.02 (d, J = 8.2 Hz, 1H), 6.86 (d, J = 8.2 Hz, 1H), 2.35-2.29 (m, 1H), 1.87 (s, 3H), 1.79 (s, 3H), 1.19-1.11 (m, 4H). |
| | (S)-3-chloro-2-(2- | | |
| | cyclopropylpyrimidin-5-yl)-5-(3- | | |
| | hydroxy-2,6-dimethylphenyl)- | | |
| | 1H-pyrrolo[2,3-b]pyridine-4- | | |
| | carbonitrile | | |
| 58 | 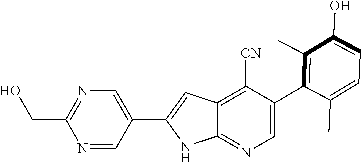 | 372.2 | (400 MHz, DMSO-d₆) δ 13.13 (s, 1H), 9.46 (s, 2H), 9.35 (s, 1H), 8.22 (s, 1H), 7.48 (s, 1H), 7.02 (d, J = 8.4 Hz, 1H), 6.85 (d, J = 8.0 Hz, 1H), 5.41 (s, 1H), 4.68 (d, J = 6.4 Hz, 2H), 1.88 (s, 3H), 1.80 (s, 3H). |
| | (S)-5-(3-hydroxy-2,6- | | |
| | dimethylphenyl)-2-(2- | | |
| | (hydroxymethyl)pyrimidin-5-yl)- | | |
| | 1H-pyrrolo[2,3-b]pyridine-4- | | |
| | carbonitrile | | |
| 59 | 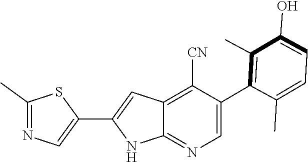 | 361.1 | (400 MHz, DMSO-d₆) δ 13.04 (s, 1H), 9.36 (s, 1H), 8.32 (s, 1H), 8.15 (s, 1H), 7.02 (d, J = 8.2 Hz, 1H), 6.94 (s, 1H), 6.85 (d, J = 8.2 Hz, 1H), 2.74 (s, 3H), 1.87 (s, 3H), 1.79 (s, 3H). |
| | (R)-5-(3-hydroxy-2,6- | | |
| | dimethylphenyl)-2-(2- | | |
| | methylthiazol-5-yl)-1H- | | |
| | pyrrolo[2,3-b]pyridine-4- | | |
| | carbonitrile | | |
| 60 | 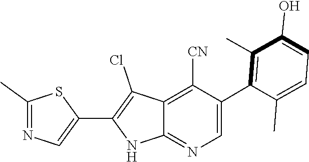 | 394.9 | (400 MHz, DMSO-d₆) δ 13.38 (s, 1H), 9.37 (s, 1H), 8.46 (s, 1H), 8.26 (s, 1H), 7.02 (d, J = 8.2 Hz, 1H), 6.85 (d, J = 8.2 Hz, 1H), 2.78 (s, 3H), 1.87 (s, 3H), 1.79 (s, 3H). |
| | (R)-3-chloro-5-(3-hydroxy-2,6- | | |
| | dimethylphenyl)-2-(2- | | |
| | methylthiazol-5-yl)-1H- | | |
| | pyrrolo[2,3-b]pyridine-4- | | |
| | carbonitrile | | |
| 61 | 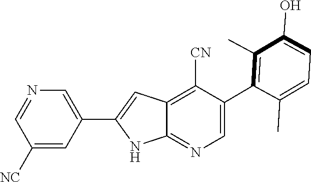 | 407.0 [M + H + CH₃CN]⁺ | (400 MHz, DMSO-d₆) δ 13.11 (s, 1H), 9.55 (d, J = 2.0 Hz, 1H), 9.36 (s, 1H), 9.05 (d, J = 1.6 Hz, 1H), 9.00 (s, 1H), 8.25 (s, 1H), 7.56 (s, 1H), 7.02 (d, J = 8.4 Hz, 1H), 6.85 (d, J = 8.4 Hz, 1H), 1.88 (s, 3H), 1.80 (s, 3H). |
| | (R)-2-(5-cyanopyridin-3-y1)-5-(3- | | |
| | hydroxy-2,6-dimethylphenyl)- | | |
| | 1H-pyrrolo[2,3-b]pyridine-4- | | |
| | carbonitrile | | |
| 62 | 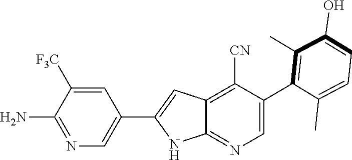 | 465.3 [M + H + CH₃CN]⁺ | (400 MHz, DMSO-d₆) δ 12.78 (s, 1H), 9.34 (s, 1H), 8.95 (s, 1H), 8.48 (s, 1H), 8.07 (s, 1H), 7.21 (s, 1H), 7.03-6.81 (m, 4H), 1.87 (s, 3H), 1.79 (s, 3H). |
| | (R)-2-(6-amino-5- | | |
| | (trifluoromethyl)pyridin-3-yl)-5- | | |
| | (3-hydroxy-2,6-dimethylphenyl)- | | |
| | 1H-pyrrolo[2,3-b]pyridine-4- | | |
| | carbonitrile | | |
| 63 | 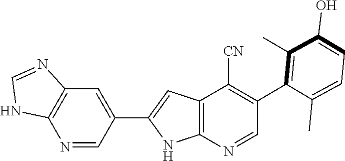 | 381.0 | (400 MHz, DMSO-d₆) δ 12.68 (s, 2H), 9.35 (s, 1H), 9.11 (s, 1H), 8.69 (s, 1H), 8.55 (s, 1H), 8.14 (s, 1H), 7.32 (s, 1H), 7.02 (d, J = 8.0 Hz, 1H), 6.85 (d, J = 8.4 Hz, 1H), 1.89 (s, 3H), 1.81 (s, 3H). |
| | (R)-5-(3-hydroxy-2,6- | | |
| | dimethylphenyl)-2-(3H- | | |
| | imidazo[4,5-b]pyridin-6-y1)-1H- | | |
| | pyrrolo[2,3-b]pyridine-4- | | |
| | carbonitrile | | |
| 64 | 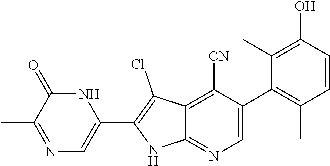 [SMILES: Cc1ccc(O)c(C)c1-c1cnc2[nH]c(-c3cnc(C)c(=O)[nH]3)c(Cl)c2c1C#N] | 406.0 | (400 MHz, DMSO-d₆) δ 13.14 (s, 1H), 9.39 (s, 1H), 8.33 (s, 1H), 7.02 (d, J = 8.4 Hz, 1H), 6.86 (d, J = 8.4 Hz, 1H), 2.43 (s, 3H), 1.87 (s, 3H), 1.79 (s, 3H). |
| | 3-chloro-5-(3-hydroxy-2,6- | | |
| | dimethylphenyl)-2-(5-methyl-6- | | |
| | oxo-1,6-dihydropyrazin-2-yl)- | | |
| | 1H-pyrrolo[2,3-b]pyridine-4- | | |
| | carbonitrile | | |
| 65 | 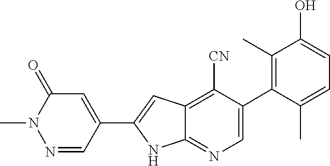 [SMILES: Cc1ccc(O)c(C)c1-c1cnc2[nH]c(-c3cnn(C)c(=O)c3)cc2c1C#N] | 372.4 | (400 MHz, DMSO-d₆) δ 13.11 (s, 1H), 9.38 (s, 1H), 8.68 (s, 1H), 8.30 (s, 1H), 7.65 (s, 1H), 7.56 (s, 1H), 7.02 (d, J = 8.4 Hz, 1H), 6.85 (d, J = 8.0 Hz, 1H), 3.70 (s, 3H), 1.87 (s, 3H), 1.79 (s, 3H). |
| | 5-(3-hydroxy-2,6- | | |
| | dimethylphenyl)-2-(1-methyl-6- | | |
| | oxo-1,6-dihydropyridazin-4-yl)- | | |
| | 1H-pyrrolo[2,3-b]pyridine-4- | | |
| | carbonitrile | | |
| 66 | 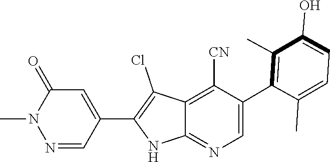 | 406.0 | (400 MHz, DMSO-d₆) δ 13.85- 12.99 (m, 1H), 9.39 (s, 1H), 8.50 (s, 1H), 8.36 (s, 1H), 7.48 (s, 1H), 7.02 (d, J = 8.0 Hz, 1H), 6.86 (d, J = 8.4 Hz, 1H), 3.73 (s, 3H), 1.87 (s, 3H), 1.79 (s, 3H). |
| | (R)-3-chloro-5-(3-hydroxy-2,6- | | |
| | dimethylphenyl)-2-(1-methyl-6- | | |
| | oxo-1,6-dihydropyridazin-4-yl)- | | |
| | 1H-pyrrolo[2,3-b]pyridine-4- | | |
| | carbonitrile | | |
| 67 | 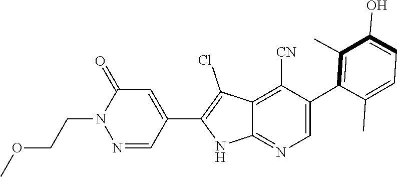 | 450.1 | (400 MHz, DMSO-d₆) δ 13.47 (s, 1H), 9.41 (s, 1H), 8.51 (d, J = 2.4 Hz, 1H), 8.40 (s, 1H), 7.47 (d, J = 2.4 Hz, 1H), 7.03 (d, J = 8.4 Hz, 1H), 6.87 (d, J = 8.4 Hz, 1H), 4.31 (t, J = 5.6 Hz, 2H), 3.75 (t, J = 5.6 Hz, 2H), 3.27 (s, 3H), 1.87 (s, 3H), 1.79 (s, 3H). |
| | (R)-3-chloro-5-(3-hydroxy-2,6- | | |
| | dimethylphenyl)-2-(1-(2- | | |
| | methoxyethyl)-6-oxo-1,6- | | |
| | dihydropyridazin-4-yl)-1H- | | |
| | pyrrolo[2,3-b]pyridine-4- | | |
| | carbonitrile | | |
| 68 | 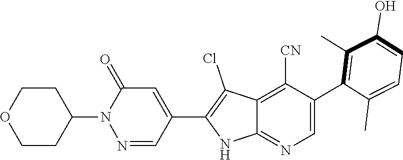 | 476.5 | (400 MHz, DMSO-d₆) δ 13.46 (s, 1H), 9.40 (s, 1H), 8.58 (d, J = 2.4 Hz, 1H), 8.39 (s, 1H), 7.48 (d, J = 2.4 Hz, 1H), 7.03 (d, J = 8.8 Hz, 1H), 6.86 (d, J = 8.0 Hz, 1H), 5.11-4.99 (m, 1H), 4.00 (t, J = 6.4 Hz, 2H), 3.52 (t, J = 11.2 Hz, 2H), 1.99-1.94 (m, 2H), 1.87 (s, 3H), 1.83-1.80 (m, 2H), 1.79 (s, 3H). |
| | (R)-3-chloro-5-(3-hydroxy-2,6- | | |
| | dimethylphenyl)-2-(6-oxo-1- | | |
| | (tetrahydro-2H-pyran-4-yl)-1,6- | | |
| | dihydropyridazin-4-yl)-1H- | | |
| | pyrrolo[2,3-b]pyridine-4- | | |
| | carbonitrile | | |
| 69 | 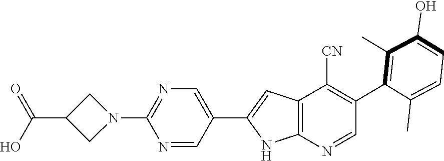 | 441.5 | (400 MHz, DMSO-d₆) δ 12.84 (s, 1H), 9.04 (s, 2H), 8.09 (s, 1H), 7.14 (d, J = 2.0 Hz, 1H), 7.01 (d, J = 8.2 Hz, 1H), 6.84 (d, J = 8.2 Hz, 1H), 4.33 (t, J = 9.0 Hz, 2H), 4.18 (dd, J = 9.0, 5.8 Hz, 2H), 1.87 (s, 3H), 1.79 (s, 3H). |
| | (R)-1-(5-(4-cyano-5-(3-hydroxy- | | |
| | 2,6-dimethylphenyl)-1H- | | |
| | pyrrolo[2,3-b]pyridin-2- | | |
| | yl)pyrimidin-2-yl)azetidine-3- | | |
| | carboxylic acid | | |
| 70 | 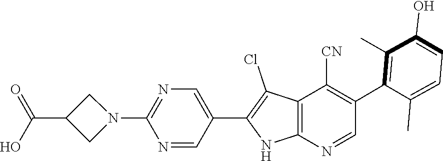 | 475.5 | (400 MHz, DMSO-d₆) δ 13.16 (s, 1H), 9.40 (s, 1H), 8.89 (d, J = 14.8 Hz, 2H), 8.22 (d, J = 4.0 Hz, 1H), 7.02 (d, J = 8.4 Hz, 1H), 6.85 (d, J = 8.4 Hz, 1H), 4.35 (t, J = 9.0 Hz, 2H), 4.25-4.10 (m, 2H), 3.63-3.58 (m, 1H), 1.87 (s, 3H), 1.79 (s, 3H). |
| | (R)-1-(5-(3-chloro-4-cyano-5-(3- | | |
| | hydroxy-2,6-dimethylphenyl)- | | |
| | 1H-pyrrolo[2,3-b]pyridin-2- | | |
| | yl)pyrimidin-2-yl)azetidine-3- | | |
| | carboxylic acid | | |
| 71 | 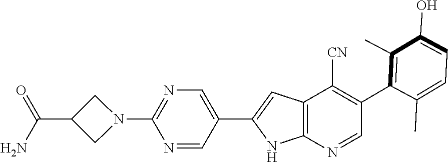 | 440.2 | (400 MHz, DMSO-d₆) δ 12.83 (s, 1H), 9.35 (s, 1H), 9.03 (s, 2H), 8.08 (s, 1H), 7.55 (s, 1H), 7.11 (d, J = 13.6 Hz, 2H), 7.00 (d, J = 8.4 Hz, 1H), 6.83 (d, J = 8.4 Hz, 1H), 4.23 (dd, J = 8.0, 4.0 Hz, 2H), 4.12 (dd, J = 8.0, 4.0 Hz, 2H), 3.48- 3.43 (m, 1H), 1.86 (s, 3H), 1.79 (s, 3H). |
| | (R)-1-(5-(4-cyano-5-(3-hydroxy- | | |
| | 2,6-dimethylphenyl)-1H- | | |
| | pyrrolo[2,3-b]pyridin-2- | | |
| | yl)pyrimidin-2-yl)azetidine-3- | | |
| | carboxamide | | |
| 72 | 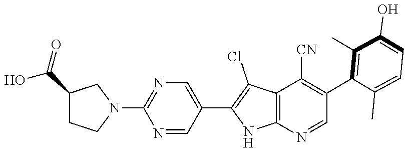 | 489.2 | (400 MHz, DMSO-d₆) δ 13.11 (s, 1H), 9.36 (s, 1H), 8.90 (s, 2H), 8.20 (s, 1H), 7.01 (d, J = 8.4 Hz, 1H), 6.84 (d, J = 8.0 Hz, 1H), 3.78 (d, J = 7.2 Hz, 2H), 3.68-3.57 (m, 2H), 3.26-3.20 (m, 2H), 2.29-2.13 (m, 2H), 1.86 (s, 3H), 1.78 (s, 3H). |
| | (R)-1-(5-((R)-3-chloro-4-cyano- | | |
| | 5-(3-hydroxy-2,6- | | |
| | dimethylphenyl)-1H-pyrrolo[2,3- | | |
| | b]pyridin-2-yl)pyrimidin-2- | | |
| | yl)pyrrolidine-3-carboxylic acid | | |
| 73 | 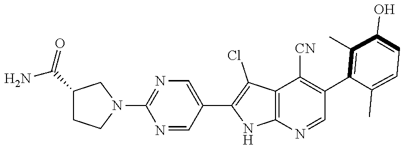 | 488.1 | (400 MHz, DMSO-d₆) δ 9.38 (s, 1H), 8.91 (s, 2H), 8.17 (s, 1H), 7.52 (s, 1H), 7.03-6.96 (m, 2H), 6.84 (d, J = 8.4 Hz, 1H), 3.82-3.64 (m, 4H), 2.2-2.08 (m, 2H), 2.05- 1.98 (m, 1H), 1.86 (s, 3H), 1.79 (s, 3H). |
| | (S)-1-(5-((R)-3-chloro-4-cyano-5- | | |
| | (3-hydroxy-2,6-dimethylphenyl)- | | |
| | 1H-pyrrolo[2,3-b]pyridin-2- | | |
| | yl)pyrimidin-2-yl)pyrrolidine-3- | | |
| | carboxamide | | |
| 74 | 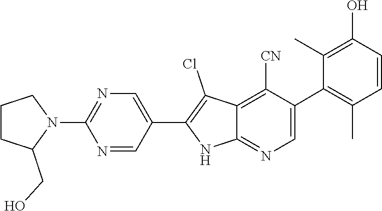 [SMILES: Cc1ccc(O)c(C)c1-c1cnc2[nH]c(-c3cnc(N4CCCC4CO)nc3)c(Cl)c2c1C#N] | 475.1 | (400 MHz, DMSO-d₆) δ 9.35 (s, 1H), 8.90 (s, 2H), 8.43 (s, 1H), 8.16 (s, 1H), 7.01 (d, J = 8.4 Hz, 1H), 6.84 (d, J = 8.4 Hz, 1H), 4.82 (s, 1H), 4.20 (s, 1H), 3.69-3.50 (m, 4H), 2.11-1.93 (m, 4H), 1.87 (s, 3H), 1.79 (s, 3H). |
| | 3-chloro-5-(3-hydroxy-2,6- | | |
| | dimethylphenyl)-2-(2-(2- | | |
| | (hydroxymethyl)pyrrolidin-1- | | |
| | yl)pyrimidin-5-yl)-1H- | | |
| | pyrrolo[2,3-b]pyridine-4- | | |
| | carbonitrile | | |
| 75 | 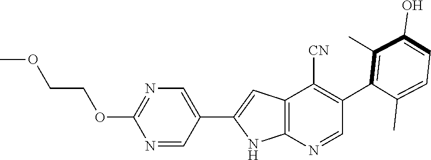 | 416.2 | (400 MHz, DMSO-d₆) δ 13.03 (s, 1H), 9.41 (s, 1H), 9.28 (s, 2H), 8.16 (s, 1H), 7.31 (s, 1H), 7.01 (d, J = 8.2 Hz, 1H), 6.85 (d, J = 8.2 Hz, 1H), 4.54-4.50 (m, 2H), 3.73- 3.70 (m, 2H), 3.33 (s, 3H), 1.87 (s, 3H), 1.79 (s, 3H). |
| | (R)-5-(3-hydroxy-2,6- | | |
| | dimethylphenyl)-2-(2-(2- | | |
| | methoxyethoxy)pyrimidin-5-yl)- | | |
| | 1H-pyrrolo[2,3-b]pyridine-4- | | |
| | carbonitrile | | |
| 76 | 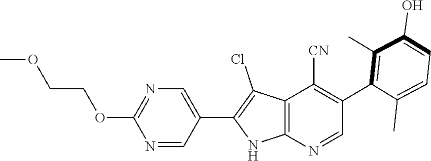 | 450.1 | (400 MHz, DMSO-d₆) δ 9.38 (s, 1H), 9.15 (s, 2H), 8.38 (s, 1H), 8.22 (s, 1H), 7.01 (d, J = 8.2 Hz, 1H), 6.85 (d, J = 8.2 Hz, 1H), 4.55-4.53 (m, 2H), 3.74-3.71 (m, 2H), 3.33 (s, 3H), 1.87 (s, 3H), 1.79 (s, 3H). |
| | (R)-3-chloro-5-(3-hydroxy-2,6- | | |
| | dimethylphenyl)-2-(2-(2- | | |
| | methoxyethoxy)pyrimidin-5-yl)- | | |
| | 1H-pyrrolo[2,3-b]pyridine-4- | | |
| | carbonitrile | | |
| 77 | 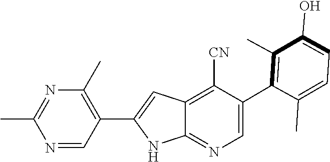 | 370.1 | (400 MHz, MeOD-d₄) δ 8.82 (s, 1H), 8.18 (s, 1H), 7.02 (d, J = 8.4 Hz, 1H), 6.93 (s, 1H), 6.83 (d, J = 8.4 Hz, 1H), 2.74 (s, 3H), 2.73 (s, 3H), 1.93 (s, 3H), 1.89 (s, 3H). |
| | (R)-2-(2,4-dimethylpyrimidin-5- | | |
| | yl)-5-(3-hydroxy-2,6- | | |
| | dimethylphenyl)-1H-pyrrolo[2,3- | | |
| | b]pyridine-4-carbonitrile | | |
| 78 | 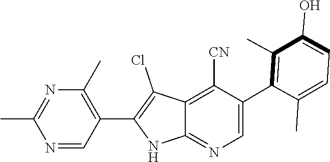 | 404.1 | (400 MHz, DMSO-d₆) δ 9.39 (s, 1H), 8.79 (s, 1H), 8.38 (s, 1H), 8.28 (s, 1H), 7.02 (d, J = 8.4 Hz, 1H), 6.86 (d, J = 8.4 Hz, 1H), 2.70 (s, 6H), 1.88 (s, 3H), 1.80 (s, 3H). |
| | (R)-3-chloro-2-(2,4- | | |
| | dimethylpyrimidin-5-yl)-5-(3- | | |
| | hydroxy-2,6-dimethylphenyl)- | | |
| | 1H-pyrrolo[2,3-b]pyridine-4- | | |
| | carbonitrile | | |
| 79 | 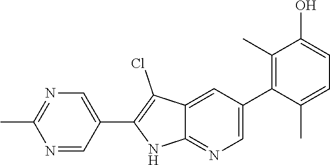 | 406.1 [M + H + CH₃CN]⁺ | (400 MHz, DMSO-d₆) δ 12.71 (s, 1H), 9.21 (d, J = 11.2 Hz, 3H), 8.11 (d, J = 2.0 Hz, 1H), 7.70 (d, J = 1.6 Hz, 1H), 6.95 (d, J = 8.0 Hz, 1H), 6.78 (d, J = 8.4 Hz, 1H), 2.72 (s, 3H), 1.88 (s, 3H), 1.81 (s, 3H). |
| | 3-(3-chloro-2-(2- | | |
| | methylpyrimidin-5-yl)-1H- | | |
| | pyrrolo[2,3-b]pyridin-5-yl)-2,4- | | |
| | dimethylphenol | | |
| 80 | 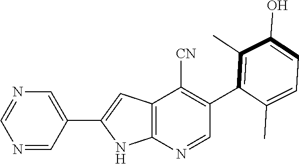 | 342.0 | (400 MHz, DMSO-d₆) δ 13.12 (s, 1H), 9.48 (s, 2H), 9.37 (s, 1H), 9.22 (s, 1H), 8.24 (s, 1H), 7.50 (s, 1H), 7.02 (d, J = 8.4 Hz, 1H), 6.85 (d, J = 8.4 Hz, 1H), 1.88 (s, 3H), 1.80 (s, 3H). |
| | (R)-5-(3-hydroxy-2,6- | | |
| | dimethylphenyl)-2-(pyrimidin-5- | | |
| | yl)-1H-pyrrolo[2,3-b]pyridine-4- | | |
| | carbonitrile | | |
| 81 | 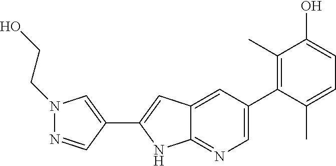 [SMILES: Cc1ccc(O)c(C)c1-c1cnc2[nH]c(-c3cnn(CCO)c3)cc2c1] | 349.2 | (400 MHz, DMSO-d₆) δ 12.05 (s, 1H), 9.15 (s, 1H), 8.26 (s, 1H), 8.02 (s, 1H), 7.86 (d, J = 2.0 Hz, 1H), 7.63 (s, 1H), 6.93 (d, J = 8.0 Hz, 1H), 6.75 (d, J = 8.4 Hz, 1H), 6.61 (d, J = 2.0 Hz, 1H), 4.23-4.18 (m, 2H), 3.81-3.76 (m, 2H), 1.88 (s, 3H), 1.81 (s, 3H). |
| | 3-(2-(1-(2-hydroxyethyl)-1H- | | |
| | pyrazol-4-yl)-1H-pyrrolo[2,3- | | |
| | b]pyridin-5-yl)-2,4- | | |
| | dimethylphenol | | |

### Example 82: Preparation of (R)-3-chloro-5-(3-hydroxy-2,6-dimethylphenyl)-2-(2-(2-oxooxazolidin-3-yl)pyrimidin-5-yl)-1H-pyrrolo[2,3-b]pyridine-4-carbonitrile

The title compound was prepared according to the following synthetic procedures.

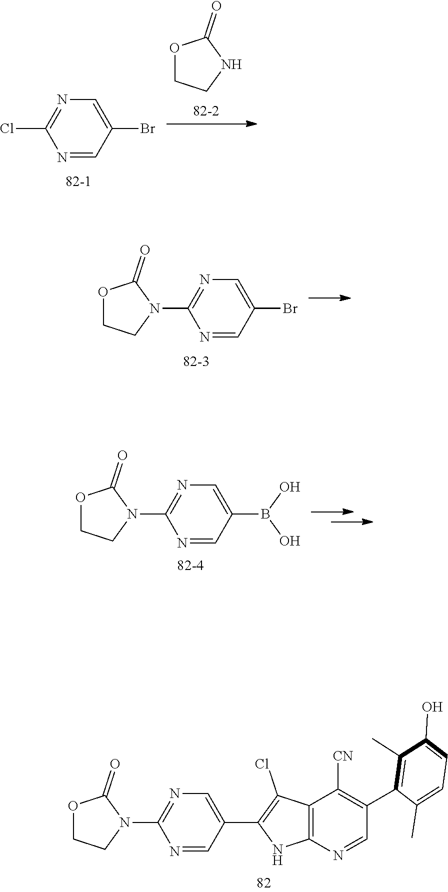

### Step 1: Preparation of Compound 82-3

To a solution of compound 82-1 (1.1 g, 5.687 mmol) in NMP (20 mL) was added compound 82-2 (449.14 mg, 5.158 mmol), and the reaction was stirred at 80° C. under N₂ for 18 h. The reaction mixture was diluted with water (100 mL) and extracted with EA (100 mL×3). The combined organic layer was washed with brine (100 mL), dried over Na₂SO₄, filtered, and concentrated in vacuo. The residue was purified by silica gel column chromatography to afford compound 82-3 (500 mg, 2.049 mmol, 37.74%). LCMS: 244.0 [M+H]⁺.

### Step 2: Preparation of Compound 82-4

To a solution of compound 82-3 (500 mg, 2.049 mmol) in dioxane (3 mL) were added 4,4,5,5-tetramethyl-2-(4,4,5,5-tetramethyl-1,3,2-dioxaborolan-2-yl)-1,3,2-dioxaborolane (1.30 g, 5.122 mmol), KOAc (603.20 mg, 6.146 mmol), and Pd(dppf)Cl₂ (224.86 mg, 0.307 mmol), and the reaction was stirred at 90 [symbol] under N₂ for 3 h. The reaction mixture was filtered and concentrated to dryness. The residue was purified by inversed phase chromatography to afford compound 82-4 (300 mg, 1.436 mmol, 70.07%). LCMS: 209.9 [M+H]⁺.

### Step 3: Preparation of Example 82

The example 82 was prepared from compound 82-4 according to the methods described in example 32-33. Spectrum data of example 82: LCMS: 461.0 [M+H]⁺; ¹H NMR: (400 MHz, DMSO-d₆) δ 13.40 (s, 1H), 10.48 (s, 1H), 9.42 (d, J=12.0 Hz, 1H), 9.29 (d, J=8.8 Hz, 1H), 9.26-9.20 (m, 1H), 8.33 (d, J=4.8 Hz, 1H), 7.03 (d, J=8.2 Hz, 1H), 6.87 (d, J=8.2 Hz, 1H), 4.78 (t, J=9.6 Hz, 2H), 4.02 (t, J=9.6 Hz, 2H), 1.87 (s, 3H), 1.79 (s, 3H).

### Example 83: Preparation of (5R)-2-(2-(2,3-dihydroxypropoxy)pyrimidin-5-yl)-5-(3-hydroxy-2,6-dimethylphenyl)-1H-pyrrolo[2,3-b]pyridine-4-carbonitrile

The title compound was prepared according to the following synthetic procedures.

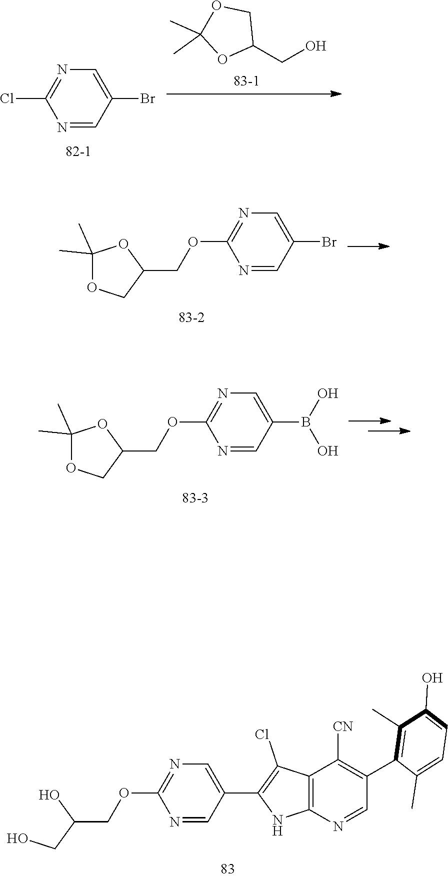

### Step 1: Preparation of Compound 83-2

To a solution of compound 83-1 (512.43 mg, 3.877 mmol) in THF (10 mL) was added NaH (124.08 mg, 3.102 mmol) at 0° C. for 0.5 h, then compound 82-1 (500 mg, 2.585 mmol) was added. The mixture was stirred at rt for 4 h. The reaction mixture was diluted with water (20 mL) and extracted with EA (30 mL×3). The combined organic layer was washed with brine (30 mL), dried over Na₂SO₄, filtered, and concentrated to dryness. The residue was purified by silica gel column chromatography to afford compound 83-2 (550 mg, 1.902 mmol, 73.6%). LCMS: 291.1 [M+H]⁺.

### Step 2: Preparation of Compound 83-3

To a solution of compound 83-2 (300 mg, 1.038 mmol) and 4,4,5,5-tetramethyl-2-(4,4,5,5-tetramethyl-1,3,2-dioxaborolan-2-yl)-1,3,2-dioxaborolane (790.4 mg, 3.113 mmol) in dioxane (5 mL) were added KOAc (305.4 mg, 3.113 mmol) and Pd(dppf)Cl₂ (75.9 mg, 0.104 mmol). The reaction mixture was replaced gas three times. The reaction mixture was stirred at 90° C. under N₂ overnight. The reaction mixture was diluted with water (30 mL) and extracted with EA (30 mL×3). The combined organic layer was washed with brine (50 mL×2), dried over Na₂SO₄, filtered and concentrated. The residue was purified by silica gel column chromatography to afford compound 83-3 (130 mg, 1.200 mmol, 26.0%). LCMS: 255.2 [M+H]⁺.

### Step 3: Preparation of Example 83

The example 83 was prepared from compound 83-3 according to the methods described in example 32-33. Spectrum data of example 83: LCMS: 432.2 [M+H]⁺; ¹H NMR: (400 MHz, DMSO-d₆) δ 12.88 (s, 1H), 9.37 (s, 1H), 9.26 (d, J=9.2 Hz, 2H), 8.17 (s, 1H), 7.32 (d, J=5.2 Hz, 1H), 7.01 (d, J=8.4 Hz, 1H), 6.85 (d, J=8.4 Hz, 1H), 4.98-4.94 (m, 1H), 4.72 (s, 1H), 4.43 (dd, J=11.0, 4.0 Hz, 1H), 4.30 (dd, J=11.0, 6.4 Hz, 1H), 3.81-3.76 (m, 1H), 3.48 (s, 2H), 1.87 (s, 3H), 1.80 (s, 3H).

### Example 84: Preparation of (R)-3-chloro-2-(2-(1,1-dioxidothiomorpholino)pyrimidin-5-yl)-5-(3-hydroxy-2,6-dimethylphenyl)-1H-pyrrolo[2,3-b]pyridine-4-carbonitrile

The title compound was prepared according to the following synthetic procedures.

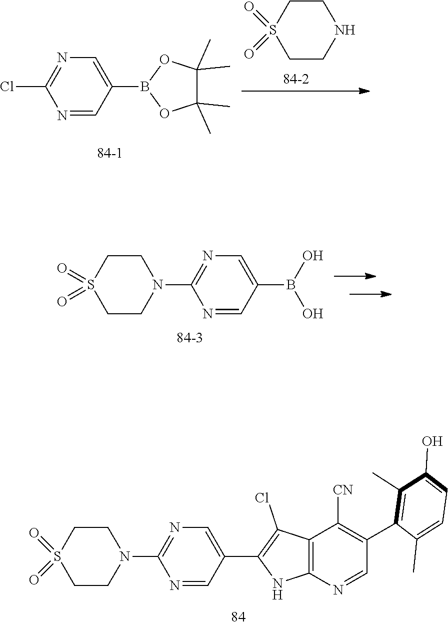

### Step 1: Preparation of Compound 84-3

To a solution of compound 84-1 (500 mg, 2.079 mmol) in ethanol (10 mL) were added compound 84-2 (843.1 mg, 6.237 mmol) and TEA (0.87 mL, 6.237 mmol), the reaction was stirred [symbol] at 80 under N₂ for 4 h. The reaction mixture was diluted with water (20 mL) and extracted with EA (30 mL×3). The combined organic layer was washed with brine (30 mL), dried over Na₂SO₄, filtered and concentrated. The residue was purified by silica gel column chromatography to afford compound 84-3 (500 mg, 1.945 mmol, 93.55%). LCMS: 258.0 [M+H]⁺.

### Step 2: Preparation of Example 84

The title compound was prepared from compound 84-3 according to the methods described in example 32-33. Spectrum data of example 84: LCMS: 509.1 [M+H]⁺; ¹H NMR: (400 MHz, DMSO-d₆) δ 9.38 (s, 1H), 9.00 (s, 2H), 8.23 (s, 1H), 7.02 (d, J=8.4 Hz, 1H), 6.85 (d, J=8.4 Hz, 1H), 4.33 (s, 4H), 3.26 (s, 4H), 1.87 (s, 3H), 1.78 (s, 3H).

### Example 85: Preparation of 2-(2-(difluoromethoxy)pyrimidin-5-yl)-5-(3-hydroxy-2,6-dimethylphenyl)-1H-pyrrolo[2,3-b]pyridine-4-carbonitrile

The title compound was prepared according to the following synthetic procedures.

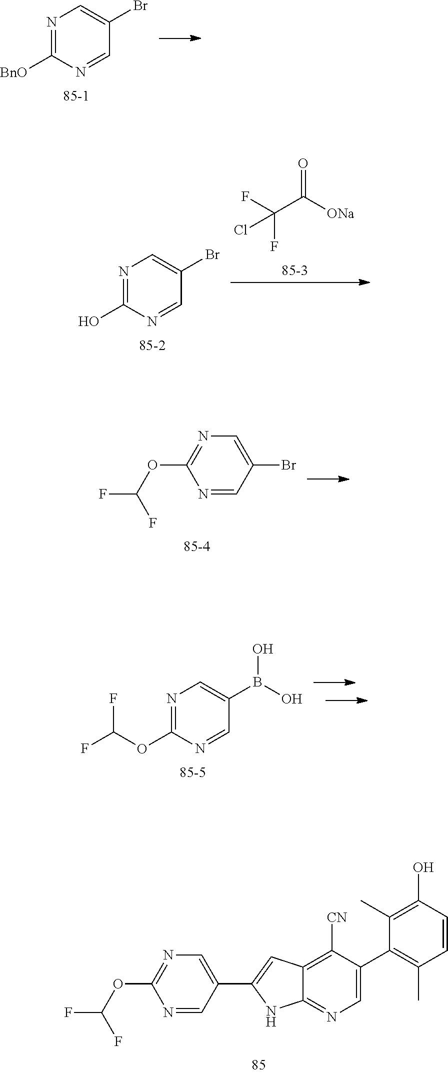

### Step 1: Preparation of Compound 85-2

A solution of compound 85-1 (5 g, 18.860 mmol) in TFA (20 mL, 18.860 mmol) was stirred at 40° C. for 16 h. The mixture was diluted with EA (100 mL) and water (100 mL) and stirred for 10 min. The mixture was filtered. The filtrate was dried under reduced pressure to afford compound 85-2 (2 g, 11.429 mmol, 60.60%).

### Step 2: Preparation of Compound 85-4

To a solution of compound 85-2 (3 g, 17.144 mmol) and K₂CO₃ (7.11 g, 51.432 mmol) in DMF (40 mL) was added compound 85-3 (7.84 g, 51.432 mmol) at 80° C. The mixture was stirred at 100° C. for 2 h. The mixture was filtered. The filtrate was diluted with EA (50 mL), which was washed with brine (50 mL×3). The organic layer was dried over Na₂SO₄, filtered and concentrated. The residue was purified by silica gel column chromatography to afford compound 85-4 (300 mg, 1.333 mmol, 7.78%).

### Step 3: Preparation of Compound 85-5

To a solution of compound 85-4 (65 mg, 0.289 mmol) in dioxane (2 mL) were added KOAc (85.06 mg, 0.867 mmol) and Pd(dppf)Cl₂ (21.14 mg, 0.029 mmol). The mixture was stirred at 100° C. for 1 h. The reaction mixture was to be used directly in next step. LCMS: 190.9 [M+H]⁺.

### Step 4: Preparation of Example 85

The example 85 was prepared from compound 85-5 according to the methods described in example 32-33. Spectrum data of example 85: LCMS: 408.0 [M+H]⁺; ¹H NMR: (400 MHz, CD₃OD) δ 9.26 (s, 2H), 8.19 (s, 1H), 7.69 (t, J=72.0 Hz, 1H), 7.28 (s, 1H), 7.04 (d, J=8.2 Hz, 1H), 6.84 (d, J=8.2 Hz, 1H), 1.95 (s, 3H), 1.90 (s, 3H).

### Example 86: Preparation of 5-(3-hydroxy-2,6-dimethylphenyl)-2-(2-(2-hydroxypropan-2-yl)pyrimidin-5-yl)-1H-pyrrolo[2,3-b]pyridine-4-carbonitrile

The title compound was prepared according to the following synthetic procedures.

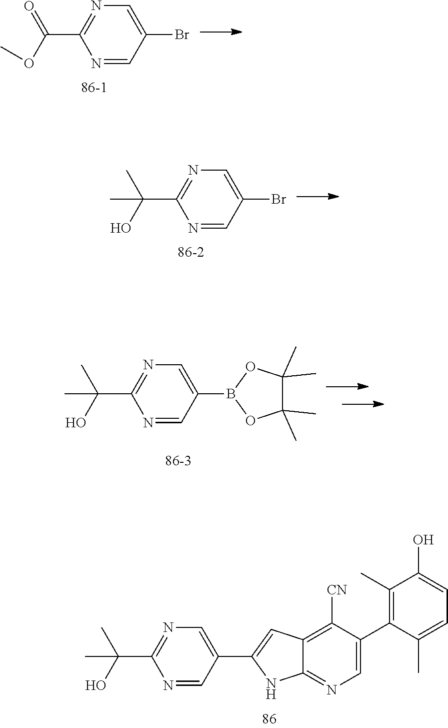 [SMILES: COC(=O)c1ncc(Br)cn1 ; CC(C)(O)c1ncc(Br)cn1 ; CC(C)(O)c1ncc(B2OC(C)(C)C(C)(C)O2)cn1 ; Cc1ccc(O)c(C)c1-c1cnc2[nH]c(-c3cnc(C(C)(C)O)nc3)cc2c1C#N]

### Step 1: Preparation of Compound 86-2

To a solution of compound 86-1 (1 g, 4.608 mmol) in THF (10 mL) was added magnesium monobromide methanide (18.431 mL, 18.431 mmol). The mixture was stirred at 0° C. for 2 h. The reaction mixture was diluted with water (20 mL), which was extracted with EA (10 mL×3). The combined organic layer was washed with brine (30 mL), dried over Na₂SO₄, filtered and concentrated. The residue was purified by silica gel column chromatography to afford compound 86-2 (400 mg, 1.843 mmol, 39.99%). LCMS: 219.0 [M+H]⁺.

### Step 2: Preparation of Compound 86-3

To a solution of compound 86-2 (100 mg, 0.461 mmol) and 4,4,5,5-tetramethyl-2-(4,4,5,5-tetramethyl-1,3,2-dioxaborolan-2-yl)-1,3,2-dioxaborolane (175.48 mg, 0.691 mmol) in dioxane (4 mL) was added KOAc (45.21 mg, 0.461 mmol) and Pd(dppf)Cl₂ (337.08 mg, 0.461 mmol). The mixture was stirred at 100° C. for 2 h. The mixture was used directly next step.

### Step 3: Preparation of Example 86

The title compound was prepared from compound 86-3 according to the methods described in example 32-33. Spectrum data of example 86: LCMS: 400.1 [M+H]⁺; ¹H NMR: (400 MHz, DMSO-d₆) δ 13.15 (s, 1H), 9.46 (s, 2H), 9.37 (s, 1H), 8.23 (s, 1H), 7.47 (s, 1H), 7.02 (d, J=8.4 Hz, 1H), 6.85 (d, J=8.4 Hz, 1H), 5.19 (s, 1H), 1.88 (s, 3H), 1.80 (s, 3H), 1.55 (s, 6H).

### Example 87: Preparation of (R)-2-(6-amino-5-ethynylpyridin-3-yl)-5-(3-hydroxy-2,6-dimethylphenyl)-1H-pyrrolo[2,3-b]pyridine-4-carbonitrile

The title compound was prepared according to the following synthetic procedures.

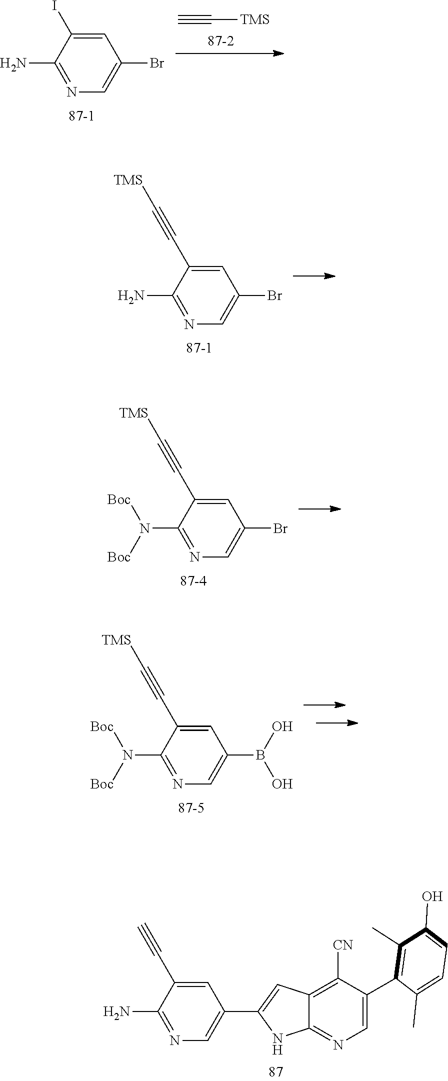

### Step 1: Preparation of Compound 87-3

To a solution of compound 87-1 (1.0 g, 3.346 mmol) and triethylamine (10 mL) in THF (10 mL) were added CuI (12.7 mg, 0.067 mmol), bis(triphenylphosphine)palladium(II) chloride (26.0 mg, 0.033 mmol) and compound 87-2 (361.4 mg, 3.680 mmol). The mixture was stirred at rt for 1 h. The mixture was filtered and concentrated, the residue was purified by silica gel column chromatography to afford compound 87-3 (870 mg, 3.232 mmol). LCMS: 310.1 [M+CH₃CN+H]⁺.

### Step 2: Preparation of Compound 87-4

To a solution of compound 87-3 (870 mg, 3.232 mmol) in DCM (25 mL) were added di-tert-butyl dicarbonate (2.23 mL, 9.695 mmol) and DMAP (39.5 mg, 0.323 mmol). The mixture was stirred at rt for 3 h. The mixture was concentrated and purified by silica gel column chromatography to afford compound 87-4 (1220 mg, 2.599 mmol). ¹H NMR (400 MHz, CDCl₃) δ 8.47 (d, J=1.2 Hz, 1H), 7.93 (d, J=1.2 Hz, 1H), 1.40 (s, 18H), 0.22 (d, J=0.6 Hz, 9H).

### Step 3: Preparation of Compound 87-5

A solution of compound 87-4 (1.35 g, 2.875 mmol), bis(pinacolato)diboron (1.46 g, 5.751 mmol), potassium acetate (846.2 mg, 8.626 mmol), Pd(dppf)Cl₂ (210.4 mg, 0.288 mmol) in dioxane (15 mL) was stirred at 90 [symbol] overnight. The mixture was filtered and concentrated, the residue was purified by silica gel column chromatography to afford compound 87-5 (920 mg, 2.118 mmol). LCMS: 435.3 [M+H]⁺.

### Step 4: Preparation of Example 87

The example 87 was prepared from compound 87-5 according to the methods described in example 32-33. Spectrum data of example 87: LCMS: 380.2 [M+H]⁺; ¹H NMR: (400 MHz, DMSO-d₆) δ 12.62 (s, 1H), 9.61 (s, 1H), 8.59 (s, 1H), 8.34 (s, 1H), 8.20 (s, 1H), 7.97 (s, 1H), 7.00 (d, J=7.4 Hz, 2H), 6.81 (d, J=8.2 Hz, 1H), 6.50 (s, 2H), 4.29 (s, 1H), 1.81 (s, 3H), 1.74 (s, 3H).

### Example 88: Preparation of 3-(2-(2-(2-hydroxypropan-2-yl)pyrimidin-5-yl)-1H-pyrrolo[2,3-b]pyridin-5-yl)-2,4-dimethylphenol

The title compound was prepared according to the following synthetic procedures.

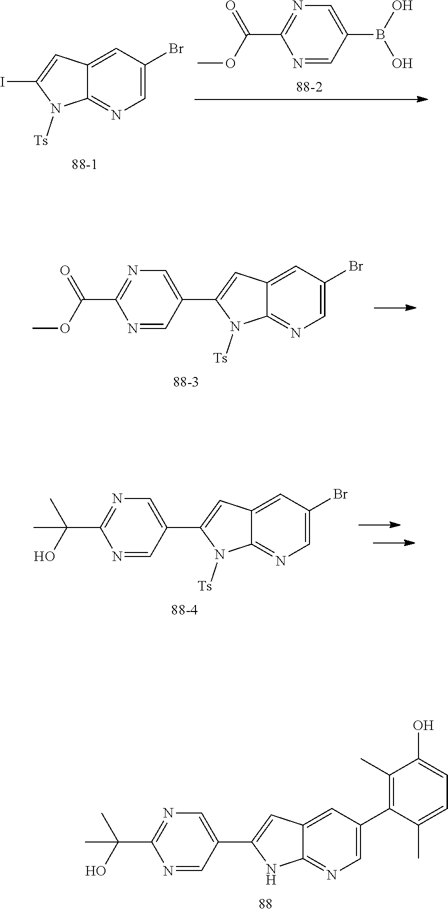

### Step 1: Preparation of Compound 88-3

To a solution of compound 88-1 (2 g, 4.192 mmol) and compound 88-2 (0.9 g, 4.192 mmol) in dioxane (20 mL) and water (4 mL) were added CsF (1.91 g, 12.576 mmol), Pd(dppf)Cl₂ (0.31 g, 0.419 mmol). The reaction was stirred at 90 [symbol] under N₂ for 16 h. The reaction was diluted with EA (60 mL) and water (50 mL). The organic layer was separated, washed with brine, and concentrated in vacuo. The residue was purified using silica gel column chromatography to afford compound 88-3 (360 mg, 0.739 mmol, 17.62%). LCMS: 489.0 [M+H]⁺.

### Step 2: Preparation of Compound 88-4

To a solution of compound 88-3 (360 mg, 0.739 mmol) in THF (5 mL) was added MeMgBr (3.7 mL, 0.739 mmol, 1M) at 0° C., and the reaction was stirred at 0° C. for 3 h. The reaction mixture was carefully added into water (30 mL) under ice-bath, which was extracted with EA (30 mL×3). The combined organic layer was washed with brine (30 mL), dried over Na₂SO₄, filtered, and concentrated to dryness. The organic layer was separated and concentrated in vacuo. The residue was purified using silica gel column chromatography to afford compound 88-4 (200 mg, 0.410 mmol, 55.55%). LCMS: 488.8 [M+H]⁺.

### Step 3: Preparation of Example 88

The example 88 was prepared from compound 88-4 according to the methods described in example 32-33. Spectrum data of example 88: LCMS: 375.1 [M+H]⁺; ¹H NMR: (400 MHz, DMSO-d₆) δ 12.45 (s, 1H), 9.35 (s, 2H), 9.15 (s, 1H), 8.00 (s, 1H), 7.75 (s, 1H), 7.16 (s, 1H), 6.94 (d, J=8.0 Hz, 1H), 6.76 (d, J=8.0 Hz, 1H), 5.12 (s, 1H), 1.89 (s, 3H), 1.82 (s, 3H), 1.54 (s, 6H).

### Example 89: Preparation of 5-(3-hydroxy-2,6-dimethylphenyl)-2-(5-(4-hydroxypiperidin-1-yl)pyridin-3-yl)-1H-pyrrolo[2,3-b]pyridine-4-carbonitrile

The title compound was prepared according to the following synthetic procedures.

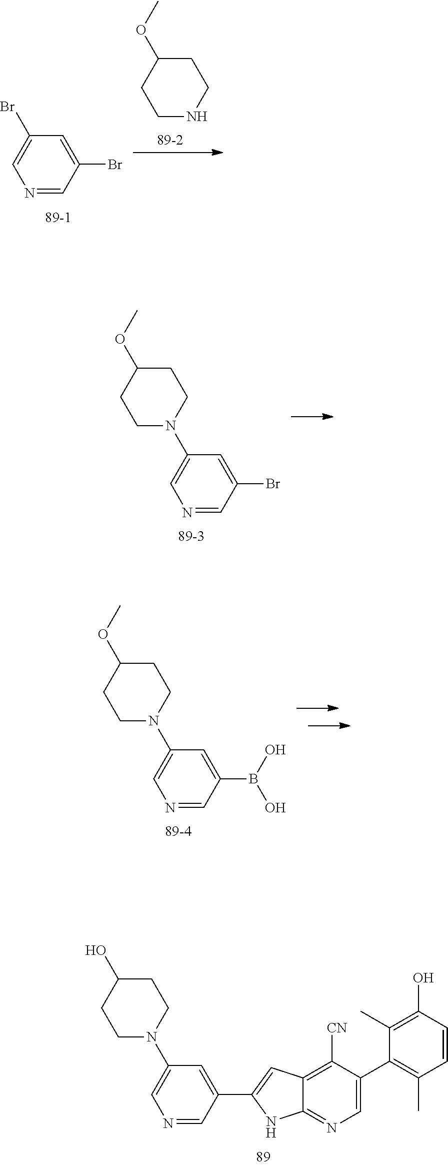

### Step 1: Preparation of Compound 89-3

To a solution of compound 89-1 (500 mg, 2.11 mmol) in DMF (10 mL) were added compound 89-2 (485 mg, 4.219 mmol) and K₂CO₃ (874 mg, 6.33 mmol) at rt. The mixture was stirred at 120° C. for 16 h. The mixture was quenched with H₂O (50 mL) and extracted with EA (60 mL×3). The organic layer was dried over Na₂SO₄ and concentrated to dryness. The residue was purified by silica gel column chromatography to afford compound 89-3 (120 mg, 0.443 mmol, 21%). LCMS: 273.1 [M+H]⁺.

### Step 2: Preparation of Compound 89-4

To a solution of compound 89-3 (50 mg, 0.185 mmol) in dioxane (1 mL) were added 4,4,5,5-tetramethyl-2-(4,4,5,5-tetramethyl-1,3,2-dioxaborolan-2-yl)-1,3,2-dioxaborolane (140 mg, 0.554 mmol), KOAc (54 mg, 0.554 mmol) and Pd(dppf)Cl₂ (18 mg, 0.024 mmol). The mixture was stirred at 90° C. under N₂ for 1.5 h. The mixture used to next step. LCMS: 237.1 [M+H]⁺.

### Step 3: Preparation of Example 89

The example 89 was prepared from compound 89-4 according to the methods described in example 32-33. Spectrum data of example 89: LCMS: 440.3 [M+H]⁺; ¹H NMR: (400 MHz, CD₃OD) δ 8.45 (s, 11H), 8.20 (s, 1H), 8.03 (s, 1H), 7.81 (s, 11H), 7.10 (s, 1H), 6.92 (d, J=8.4 Hz, 1H), 6.72 (d, J=8.4 Hz, 1H), 3.78-3.66 (m, 3H), 3.02 (t, J=10.0 Hz, 211), 1.94 (d, J 13.1 Hz, 2H), 1.84 (s, 3H), 1.79 (s, 3H), 1.62-1.57 (m, 2H).

### Example 90: Preparation of 5-(2-ethyl-3-hydroxy-6-methylphenyl)-2-(2-methylpyrimidin-5-yl)-1H-pyrrolo[2,3-b]pyridine-4-carbonitrile

The title compound was prepared according to the following synthetic procedures.

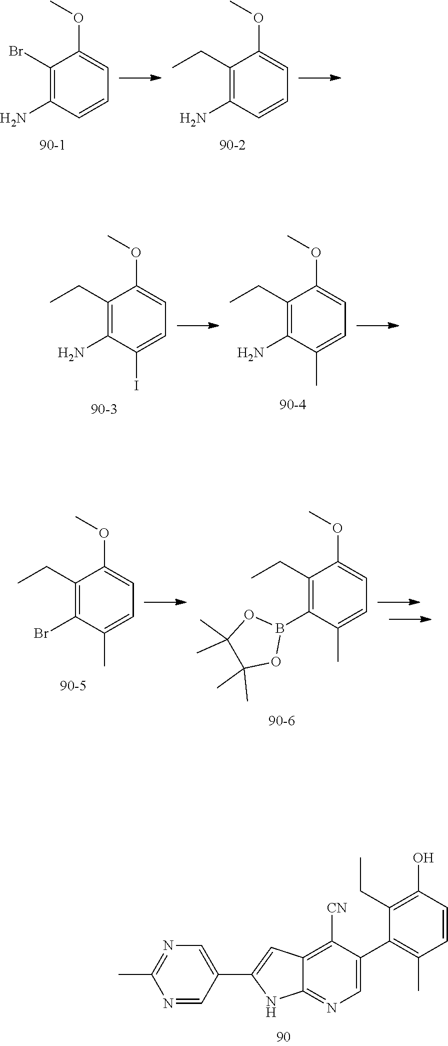

### Step 1: Preparation of Compound 90-2

To a solution of compound 90-1 (8.0 g, 39.594 mmol) and ethylboronic acid (5.9 g, 79.188 mmol) in dioxane (100 mL) and H₂O (20 mL) were added Pd(dppf)Cl₂ (2.9 g, 3.959 mmol) and K₂CO₃ (16.4 g, 118.782 mmol). The mixture was stirred at 100° C. for 12 h. The reaction mixture was diluted with water (100 mL) and extracted with EA (100 mL×3). The combined organic layer was washed with brine (30 mL), dried over Na₂SO₄, filtered, and concentrated to dryness. The residue was purified by silica gel column chromatography to afford compound 90-2 (2.4 g, 15.872 mmol, 40.09%). LCMS: 152.2 [M+H]⁺.

### Step 2: Preparation of Compound 90-3

To a solution of compound 90-2 (7.0 g, 46.293 mmol) in DMF (20 mL) was added NIS (8.0 g, 46.293 mmol). The mixture was stirred at rt for 12 h. The reaction mixture was diluted with EA (30 mL) and washed with brine (20 mL×3). The organic layer was dried over Na₂SO₄, filtered, and concentrated to dryness. The residue was purified by silica gel column chromatography to afford compound 90-3 (8 g, 28.869 mmol, 62.36%). LCMS: 278.2 [M+H]⁺.

### Step 3: Preparation of Compound 90-4

To a solution of compound 90-3 (850 mg, 3.067 mmol) and 2,4,6-trimethyl-1,3,5,2,4,6-trioxatriborinane (770 mg, 6.135 mmol) in dioxane (20 mL) and H₂O (4 mL) were added K₂CO₃ (1271 mg, 9.202 mmol) and Pd(dtbpf)Cl₂ (201 mg, 0.307 mmol). The mixture was stirred at 100° C. for 12 h. The reaction mixture was diluted with water (20 mL) and extracted with EA (20 mL×3). The combined organic layer was washed with brine (30 mL), dried over Na₂SO₄, filtered, and concentrated to dryness. The residue was purified by silica gel column chromatography to afford compound 90-4 (400 mg, 2.421 mmol, 78.92%). LCMS: 166.3 [M+H]⁺.

### Step 4: Preparation of Compound 90-5

To a solution of compound 90-4 (700 mg, 4.236 mmol) in AcOH (8 mL) and HBr (1 mL) was added dropwise of NaNO₂ (292 mg, 4.236 mmol) in H₂O (1 mL) at 0° C., the mixture was stirred at 0° C. for 2 h. Then CuBr (608 mg, 4.236 mmol) in HBr (1 mL) was added. The resulting mixture was stirred at rt for 2 h. The reaction mixture was quenched by water (10 mL) and extracted with EA (10 mL×3). The combined organic layer was washed with brine (10 mL), dried over Na₂SO₄, filtered, and concentrated to dryness. The residue was purified by silica gel column chromatography to afford compound 90-5 (300 mg, 1.309 mmol, 30.91%). ¹H NMR: (400 MHz, DMSO-d₆) δ 7.17 (t, J=9.8 Hz, 1H), 6.95-6.86 (m, 1H), 3.82-3.75 (m, 3H), 2.82-2.70 (m, 2H), 2.34-2.25 (m, 3H), 1.11-0.98 (m, 3H).

### Step 5: Preparation of Compound 90-6

To a solution of compound 90-5 (440 mg, 1.920 mmol) and 4,4,5,5-tetramethyl-2-(4,4,5,5-tetramethyl-1,3,2-dioxaborolan-2-yl)-1,3,2-dioxaborolane (732 mg, 2.881 mmol) in DMF (5 mL) was added potassium acetate (565 mg, 5.761 mmol) and Pd(dppf)Cl₂ (140 mg, 0.192 mmol). The mixture was stirred at 130° C. for 12 h. The reaction mixture was diluted with EA (20 mL), which was washed with brine (10 mL×3). The organic layer was dried over Na₂SO₄, filtered, and concentrated to dryness. The residue was purified by prep-TLC to afford compound 90-6 (260 mg, 0.941 mmol, 49.02%). ¹H NMR: (400 MHz, DMSO-d₆) δ 6.92 (d, J=8.4 Hz, 1H), 6.82 (d, J=8.4 Hz, 1H), 3.72 (s, 3H), 2.56 (q, J=7.4 Hz, 2H), 2.22 (s, 3H), 1.32 (s, 12H), 1.04 (t, J=7.4 Hz, 3H).

### Step 6: Preparation of Example 90

The title compound was prepared from compound 90-6 according to the methods described in example 32-33. Spectrum data of example 90: LCMS: 370.4 [M+H]⁺.

### Example 91: Preparation of (R)-5-(2-ethyl-3-hydroxy-6-methylphenyl)-2-(2-methylpyrimidin-5-yl)-1H-pyrrolo[2,3-b]pyridine-4-carbonitrile

### Step 1: Preparation of Example 91

The title compound was separated by prep-SFC from example 90. LCMS: 411.2[M+H+CH₃CN]; 1H NMR: (400 MHz, DMSO-d₆) δ 13.08 (s, 1H), 9.37 (s, 2H), 9.33 (s, 1H), 8.22 (s, 1H), 7.43 (s, 11H), 7.01 (d, J=8.2 Hz, 11H), 6.85 (d, J=8.2 Hz, 11H), 2.70 (s, 3H), 2.51-2.19 (m, 2H), 1.84 (s, 3H), 0.87 (t, J=7.4 Hz, 3H).

### Example 92: Preparation of 2-(4-amino-2-methylpyrimidin-5-yl)-5-(3-hydroxy-2,6-dimethylphenyl)-1H-pyrrolo[2,3-b]pyridine-4-carbonitrile

The title compound was prepared according to the following synthetic procedures.

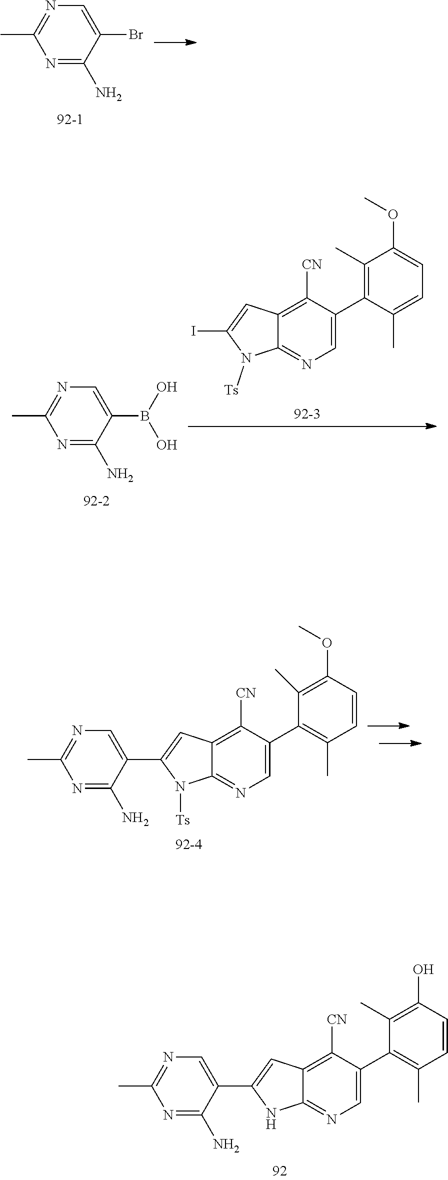

### Step 1: Preparation of Compound 92-2

To a solution of compound 92-1 (100 mg, 0.53 mmol) in dioxane (2 mL) were added B₂Pin₂ (405 mg, 1.60 mmol), KOAc (156 mg, 1.60 mmol), Pd(dppf)Cl₂ (51 mg, 0.07 mmol). The mixture was stirred at 100° C. for 3 h. The mixture was used directly in next step. LCMS: 154.2 [M+H]⁺.

### Step 2: Preparation of Compound 92-4

To the reaction mixture of step 1 were added compound 92-3 (148 mg, 0.266 mmol), CsF (121 mg, 0.80 mmol), BrettphPdG₃ (30 mg, 0.03 mmol) and water (0.4 mL), and the mixture was stirred at 75 [symbol] for 2 h. The mixture was extracted with DCM (30 mL×3). The combined organic phase was washed with brine (40 mL), dried over Na₂SO₄, filtered, concentrated, and purified by prep-TLC to afford compound 92-4 (20 mg, 0.037 mmol). LCMS: 539.3 [M+H]⁺.

### Step 3: Preparation of Example 92

The example 92 was prepared from compound 92-4 according to the methods described in example 32-33. Spectrum data of example 92: LCMS: 371.2[M+H]⁺; ¹H NMR: (400 MHz, DMSO-d₆) δ 9.33 (s, 1H), 8.45-8.39 (m, 3H), 8.09 (s, 1H), 7.17-7.03 (m, 1H), 7.00 (d, J=8.0 Hz, 1H), 6.88 (s, 1H), 6.83 (d, J=8.2 Hz, 1H), 2.40 (s, 3H), 1.87 (s, 3H), 1.79 (s, 3H).

### Example 93: Preparation of (R)-5-(3-hydroxy-2,6-dimethylphenyl)-2-(2-(pyrrolidin-1-ylmethyl)pyrimidin-5-yl)-1H-pyrrolo[2,3-b]pyridine-4-carbonitrile

The title compound was prepared according to the following synthetic procedures.

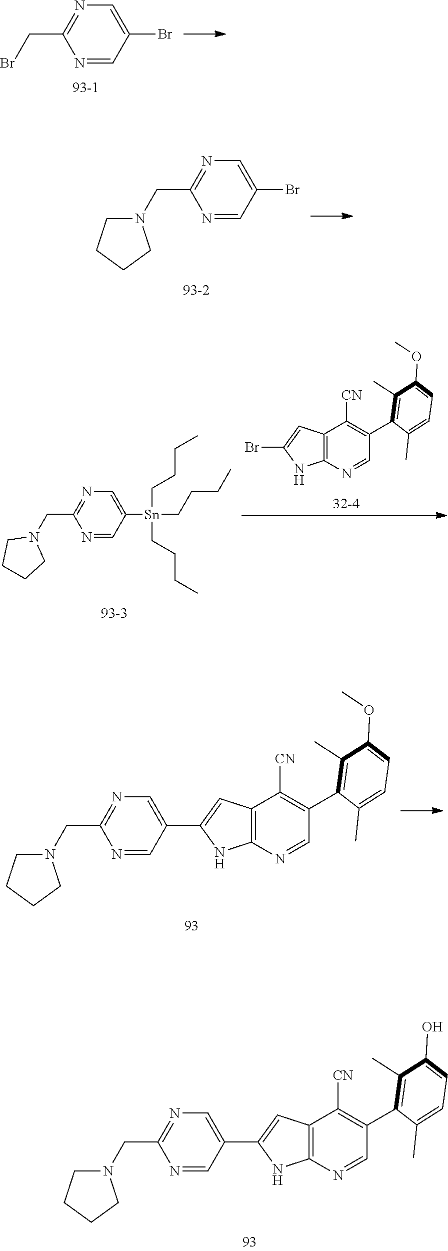

### Step 1: Preparation of Compound 93-2

To a solution of compound 93-1 (650 mg, 2.580 mmol) in CH₃CN (10 mL) were added tetrahydropyrrole (0.4 mL, 5.161 mmol), K₂CO₃ (1069.8 mg, 7.741 mmol). The mixture was stirred at room temperature for 4 h. The mixture was concentrated. The residue was purified by silica gel column chromatography to afford compound 93-2 (520 mg, 2.148 mmol, 83.23%). LCMS: 244.0 [M+H]⁺.

### Step 2: Preparation of Compound 93-3

To a solution of compound 93-2 (470 mg, 1.941 mmol) in dioxane (8 mL) were added 1,1,1,2,2,2-hexabutyldistannane (2252.2 mg, 3.882 mmol), LiCl (164.6 mg, 3.882 mmol) and Pd(PPh₃)₄ (224.3 mg, 0.194 mmol). The mixture was stirred at 100° C. for 4 h under N₂ atmosphere. The mixture was concentrated. The residue was purified by silica gel column chromatography to afford compound 93-3 (530 mg, 1.172 mmol, 60.37%). LCMS: 454.4 [M+H]⁺.

### Step 3: Preparation of Compound 93-4

To a solution of compound 32-4 (100 mg, 0.281 mmol) in dioxane (5 mL) were added compound 93-3 (253.9 mg, 0.561 mmol), LiCl (11.9 mg, 0.281 mmol) and Pd(PPh₃)₄ (32.4 mg, 0.028 mmol). The mixture was added at 100° C. for 16 h under N₂ atmosphere. The reaction mixture was diluted with DCM (30 mL), which was washed with brine (10 mL×3). The organic layer was dried over Na₂SO₄, filtered, and concentrated to dryness. The residue was purified by silica gel column chromatography to afford compound 93-4. LCMS: 439.5 [M+H]⁺.

### Step 4: Preparation of Example 93

To a solution of compound 93-4 (47 mg, 0.107 mmol) in DCM (4 mL) was added BBr₃ (134.3 mg, 0.536 mmol), the mixture was stirred at room temperature for 1 h. The reaction was quenched with MeOH (5 mL). The mixture was concentrated to dryness, purified by prep-HPLC to afford example 93. LCMS: 425.4 [M+H]⁺; ¹H NMR: (400 MHz, DMSO-d₆) δ 13.14 (s, 1H), 9.44 (s, 2H), 9.37 (s, 1H), 8.22 (s, 1H), 8.18 (s, 1H), 7.47 (s, 1H), 7.02 (d, J=8.4 Hz, 1H), 6.85 (d, J=8.4 Hz, 1H), 3.93 (s, 2H), 2.65 (t, J=5.6 Hz, 4H), 1.87 (s, 3H), 1.80 (s, 3H), 1.76-1.70 (m, 4H).

The following examples 94-95 were prepared according to the methods described in example 93 with analogous starting materials.

| | | LCMS | |
|---|---|---|---|
| Example | Structure | [M + H]⁺ | ¹H NMR |
| 94 | 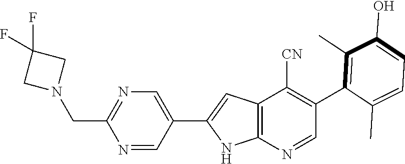 | 447.2 | (400 MHz, DMSO-d₆) δ 13.16 (s, 1H), 9.43 (s, 2H), 8.47 (s, 1H), 8.21 (s, 1H), 7.45 (s, 1H), 7.01 (d, J = 8.2 Hz, 1H), 6.85 (d, J = 8.2 Hz, 1H), 4.03 (s, 2H), 3.82 (t, J = 12.4 Hz, 4H), 1.87 (s, 3H), 1.80 (s, 3H). |
| | (R)-2-(2-((3,3- | | |
| | difluoroazetidin-1- | | |
| | yl)methyl)pyrimidin-5-yl)-5- | | |
| | (3-hydroxy-2,6- | | |
| | dimethylphenyl)-1H- | | |
| | pyrrolo[2,3-b]pyridine-4- | | |
| | carbonitrile | | |
| 95 | 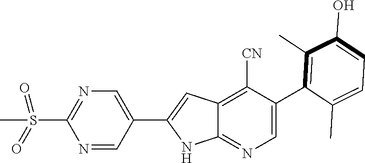 | 461.0 [M + H + CH₃CN]⁺ | (400 MHz, DMSO-d₆) δ 13.16 (s, 1H), 9.71 (s, 2H), 9.38 (s, 1H), 8.30 (s, 1H), 7.69 (s, 1H), 7.03 (d, J = 8.2 Hz, 1H), 6.86 (d, J = 8.2 Hz, 1H), 3.48 (s, 3H), 1.88 (s, 3H), 1.81 (s, 3H). |
| | (R)-5-(3-hydroxy-2,6- | | |
| | dimethylphenyl)-2-(2- | | |
| | (methylsulfonyl)pyrimidin-5- | | |
| | yl)-1H-pyrrolo[2,3- | | |
| | b]pyridine-4-carbonitrile | | |

### Example 96: Preparation of (R)-5-(3-hydroxy-2,6-dimethylphenyl)-2-(2-(methyl(oxetan-3-yl)amino)pyrimidin-5-yl)-1H-pyrrolo[2,3-b]pyridine-4-carbonitrile

The title compound was synthesized from 96-1, which was prepared according to the methods described in example 32-33 with analogous starting materials.

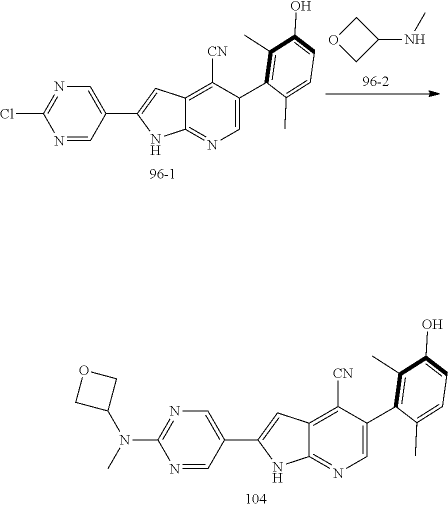

### Step 1: Preparation of Example 96

To a solution of compound 96-1 (40 mg, 0.11 mmol) in DMF (1 ml) were added compound 96-2 (19 mg, 0.21 mmol) and TEA (32 mg, 0.32 mmol), and the reaction was stirred at 50° C. overnight. The mixture was purified using Prep-HPLC and dried to afford example 96. LCMS: 427.1 [M+H]⁺; ¹H NMR: (400 MHz, DMSO-d₆) δ 12.19 (s, 1H), 9.30 (s, 1H), 8.40 (s, 1H), 7.87 (s, 1H), 7.43 (s, 1H), 6.97 (d, J=8.0 Hz, 1H), 6.81 (d, J=8.4 Hz, 11H), 6.17 (s, 1H), 4.18 (d, J=11.6 Hz, iH), 4.04 (d, J=12.0 Hz, 1H), 3.62 (s, 1H), 3.51 (s, 1H), 3.20 (d, J=8.8 Hz, 1H), 2.84 (s, 3H), 1.85 (s, 3H), 1.78 (s, 3H).

The following examples 97-103 were prepared according to the methods described in example 96 with analogous starting materials.

| | | LCMS | |
|---|---|---|---|
| Example | Structure | [M + H]⁺ | ¹H NMR |
| 97 | 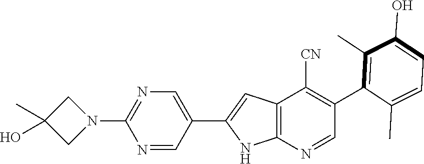 | 427.2 | (400 MHz, DMSO-d₆) δ 12.80 (s, 1H), 9.33 (s, 1H), 9.01 (s, 2H), 8.08 (s, 1H), 7.11 (s, 1H), 7.00 (d, J = 8.2 Hz, 1H), 6.84 (d, J = 8.2 Hz, 1H), 5.69 (s, 1H), 3.99 (q, J = 9.2 Hz, 4H), 1.87 (s, 3H), 1.79 (s, 3H), 1.46 (s, 3H). |
| | (R)-5-(3-hydroxy-2,6- | | |
| | dimethylphenyl)-2-(2-(3-hydroxy-3- | | |
| | methylazetidin-1-yl)pyrimidin-5-yl)- | | |
| | 1H-pyrrolo[2,3-b]pyridine-4- | | |
| | carbonitrile | | |
| 98 | 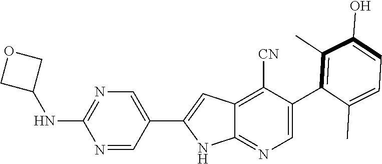 | 413.2 | (400 MHz, DMSO-d₆) δ 12.75 (s, 1H), 9.34 (s, 1H), 8.99 (s, 2H), 8.43 (d, J = 6.0 Hz, 1H), 8.08 (s, 1H), 7.11 (s, 1H), 7.00 (d, J = 8.2 Hz, 1H), 6.83 (d, J = 8.2 Hz, 1H), 5.00 (dd, J = 13.4, 7.0 Hz, 1H), 4.81 (t, J = 6.8 Hz, 2H), 4.56 (t, J = 6.4 Hz, 2H), 1.86 (s, 3H), 1.79 (s, 3H). |
| | (R)-5-(3-hydroxy-2,6- | | |
| | dimethylphenyl)-2-(2-(oxetan-3- | | |
| | ylamino)pyrimidin-5-yl)-1H- | | |
| | pyrrolo[2,3-b]pyridine-4-carbonitrile | | |
| 99 | 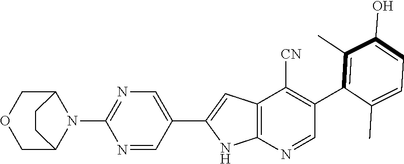 | 453.0 | (400 MHz, DMSO-d₆) δ 12.82 (s, 1H), 9.33 (s, 1H), 9.06 (s, 2H), 8.09 (s, 1H), 7.13 (s, 1H), 7.00 (d, J = 8.2 Hz, 1H), 6.84 (d, J = 8.2 Hz, 1H), 4.70 (s, 2H), 3.64 (s, 4H), 2.05-1.99 (m, 4H), 1.87 (s, 3H), 1.80 (s, 3H). |
| | (5R)-2-(2-(3-oxa-8- | | |
| | azabicyclo[3.2.1]octan-8- | | |
| | yl)pyrimidin-5-yl)-5-(3-hydroxy-2,6- | | |
| | dimethylphenyl)-1H-pyrrolo[2,3- | | |
| | b]pyridine-4-carbonitrile | | |
| 100 | 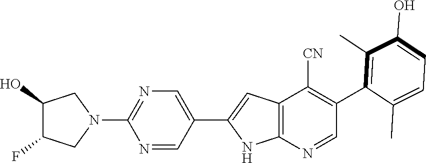 | 445.3 | (400 MHz, DMSO-d₆) δ 12.81 (s, 1H), 9.33 (s, 1H), 9.06 (s, 2H), 8.08 (s, 1H), 7.12 (s, 1H), 7.00 (d, J = 8.2 Hz, 1H), 6.84 (d, J = 8.2 Hz, 1H), 5.62 (d, J = 3.6 Hz, 1H), 5.11 (d, J = 51.0 Hz, 1H), 4.39 (s, 1H), 3.89 (s, 1H), 3.81 (d, J = 11.8 Hz, 1H), 3.71 (s, 2H), 1.87 (s, 3H), 1.80 (s, 3H). |
| | (R)-2-(2-((3S,4S)-3-fluoro-4- | | |
| | hydroxypyrrolidin-1-yl)pyrimidin-5- | | |
| | yl)-5-(3-hydroxy-2,6- | | |
| | dimethylphenyl)-1H-pyrrolo[2,3- | | |
| | b]pyridine-4-carbonitrile | | |
| 101 | 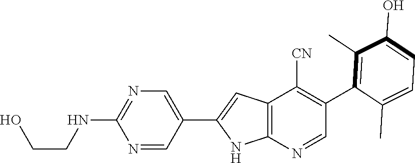 | 401.4 | (400 MHz, DMSO-d₆) δ 9.34 (s, 1H), 8.96 (s, 2H), 8.06 (s, 1H), 7.57 (t, J = 5.8 Hz, 1H), 7.08 (s, 1H), 7.00 (d, J = 8.2 Hz, 1H), 6.84 (d, J = 8.2 Hz, 1H), 4.72 (s, 1H), 3.57-3.53 (m, 2H), 3.45-3.41 (m, 2H), 1.87 (s, 3H), 1.79 (s, 3H). |
| | (R)-5-(3-hydroxy-2,6- | | |
| | dimethylphenyl)-2-(2-((2- | | |
| | hydroxyethyl)amino)pyrimidin-5- | | |
| | yl)-1H-pyrrolo[2,3-b]pyridine-4- | | |
| | carbonitrile | | |
| 102 | 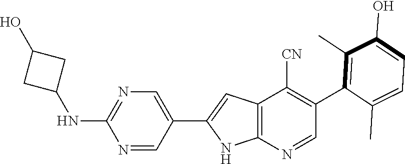 | 427.2 | (400 MHz, DMSO-d₆) δ 12.73 (s, 1H), 9.35 (s, 1H), 8.95 (d, J = 4.2 Hz, 2H), 8.06 (s, 1H), 8.01 (d, J = 6.8 Hz, 1H), 7.07 (s, 1H), 7.00 (d, J = 8.0 Hz, 1H), 6.84 (d, J = 8.4 Hz, 1H), 5.03 (d, J = 5.4 Hz, 1H), 4.41 (d, J = 7.2 Hz, 1H), 4.32 (d, J = 5.0 Hz, 1H), 2.28-2.09 (m, 4H), 1.87 (s, 3H), 1.79 (s, 3H). |
| | (R)-5-(3-hydroxy-2,6- | | |
| | dimethylphenyl)-2-(2-((3- | | |
| | hydroxycyclobutyl)amino)pyrimidin- | | |
| | 5-yl)-1H-pyrrolo[2,3-b]pyridine-4- | | |
| | carbonitrile | | |
| | (cis or trans mixture of | | |
| | hydroxycyclobutylamino part with 3- | | |
| | hydroxy-2,6-dimethylphenyl ring ) | | |
| 103 | 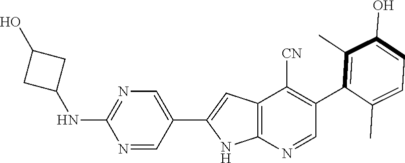 | 427.1 | (400 MHz, DMSO-d₆) δ 12.73 (s, 1H), 9.36 (s, 1H), 8.95 (s, 2H), 8.06 (s, 1H), 7.93 (d, J = 7.2 Hz, 1H), 7.07 (s, 1H), 7.00 (d, J = 8.2 Hz, 1H), 6.84 (d, J = 8.2 Hz, 1H), 5.09 (d, J = 5.8 Hz, 1H), 3.97- 3.75 (m, 2H), 2.65-2.56 (m, 2H), 1.91- 1.81 (m, 5H), 1.79 (s, 3H). |
| | (R)-5-(3-hydroxy-2,6- | | |
| | dimethylphenyl)-2-(2-((3- | | |
| | hydroxycyclobutyl)amino)pyrimidin- | | |
| | 5-yl)-1H-pyrrolo[2,3-b]pyridine-4- | | |
| | carbonitrile | | |
| | (cis or trans mixture of | | |
| | hydroxycyclobutylamino part with 3- | | |
| | hydroxy-2,6-dimethylphenyl ring ) | | |

### Example 104: Preparation of (R)-5-(3-hydroxy-2,6-dimethylphenyl)-2-(2-(prop-1-yn-1-yl)pyrimidin-5-yl)-1H-pyrrolo[2,3-b]pyridine-4-carbonitrile

The title compound was synthesized from compound 96-1, which was prepared according to the methods described in example 32 with analogous starting materials.

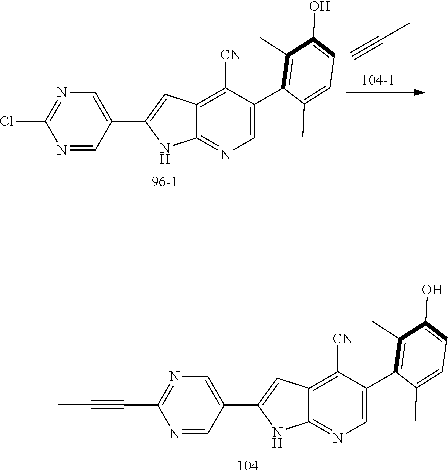

### Step 1: Preparation of Example 104

A mixture of compound 96-1 (30 mg, 0.080 mmol), compound 104-1 (0.16 mL, 1 M solution in DMF, 0.160 mmol), CuI (2.5 mg, 0.008 mmol), bis(triphenylphosphine)palladium(II) chloride (6.2 mg, 0.008 mmol), TEA (24.2 mg, 0.239 mmol) in THF (3 mL) was stirred at 50 [symbol] for 16 h. The mixture was concentrated and purified by prep-HPLC to afford example 104. LCMS: 380.2 [M+H]⁺; ¹H NMR (400 MHz, DMSO-d₆) δ 13.12 (s, 1H), 9.43 (s, 2H), 9.38 (s, 1H), 8.23 (s, 1H), 7.52 (s, 1H), 7.02 (d, J=8.3 Hz, 1H), 6.85 (d, J=8.2 Hz, 1H), 2.16 (s, 3H), 1.87 (s, 3H), 1.80 (s, 3H).

### Example 105: Preparation of (R)-5-(4-cyano-5-(3-hydroxy-2,6-dimethylphenyl)-1H-pyrrolo[2,3-b]pyridin-2-yl)pyrimidine-2-carboxamide

The title compound was synthesized from compound 105-1, which was prepared according to the methods described in example 32 with analogous starting materials.

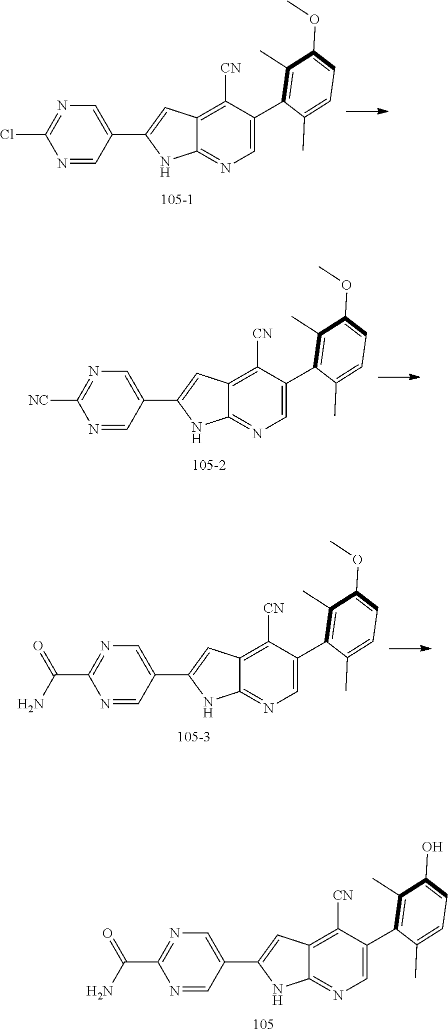

### Step 1: Preparation of Compound 105-2

To a solution of compound 105-1 (120.0 mg, 0.308 mmol) in DMSO (1.5 mL) were added 4-azabicyclo[2.2.2]octan-7-ol (7.8 mg, 0.062 mmol) and KCN (40.1 mg, 0.616 mmol). The mixture was [symbol] stirred at 50 I for 1 h. The reaction mixture was diluted with water (20 mL) and extracted with EA (30 mL×3). The combined organic layer was washed with brine (30 mL), dried over Na₂SO₄, filtered, and concentrated to dryness. The residue was purified by silica gel column chromatography to afford compound 105-2. LCMS: 381.2 [M+H]⁺.

### Step 2: Preparation of Compound 105-3

To a solution of compound 105-2 (45.0 mg, 0.118 mmol) in MeOH (3.0 mL) were added NaOH (14.2 mg, 0.355 mmol) and H₂O₂ (12.1 mg, 0.355 mmol). The mixture was stirred at room temperature overnight. The mixture was concentrated, and the residue was washed with water. The residue was purified by prep-TLC to afford compound 105-3. LCMS: 399.2 [M+H]⁺.

### Step 3: Preparation of Example 105

To a solution of compound 105-3 (22.0 mg, 0.055 mmol) in DCM (3.0 mL) was added BBr₃ (0.03 mL, 0.276 mmol) at 0 [symbol] . The mixture was stirred at 0 [symbol] for 1 h. The reaction mixture was carefully added MeOH (2 mL) while stirring and was diluted with water (10 mL). Then pH of the mixture was adjusted to 8 with NaHCO₃. The mixture was extracted with DCM (20 mL) and EA (20 mL). The combined organic layer was dried over Na₂SO₄, filtered, and concentrated to dryness. The residue was purified by prep-TLC to afford example 105. LCMS: 385.2 [M+H]⁺; ¹H NMR: (400 MHz, DMSO-d₆) δ 13.46 (s, 1H), 9.67 (s, 2H), 9.54 (s, 1H), 8.36 (s, 1H), 8.27 (s, 1H), 7.93 (s, 1H), 7.63 (s, 1H), 7.02 (d, J=8.4 Hz, 1H), 6.93 (d, J=8.4 Hz, 1H), 1.89 (s, 3H), 1.81 (s, 3H).

### Example 106: Preparation of (R)-2-(2-aminopyrimidin-5-yl)-5-(3-hydroxy-2,6-dimethylphenyl)-3-methyl-1H-pyrrolo[2,3-b]pyridine-4-carbonitrile

The title compound was prepared according to the following synthetic procedures.

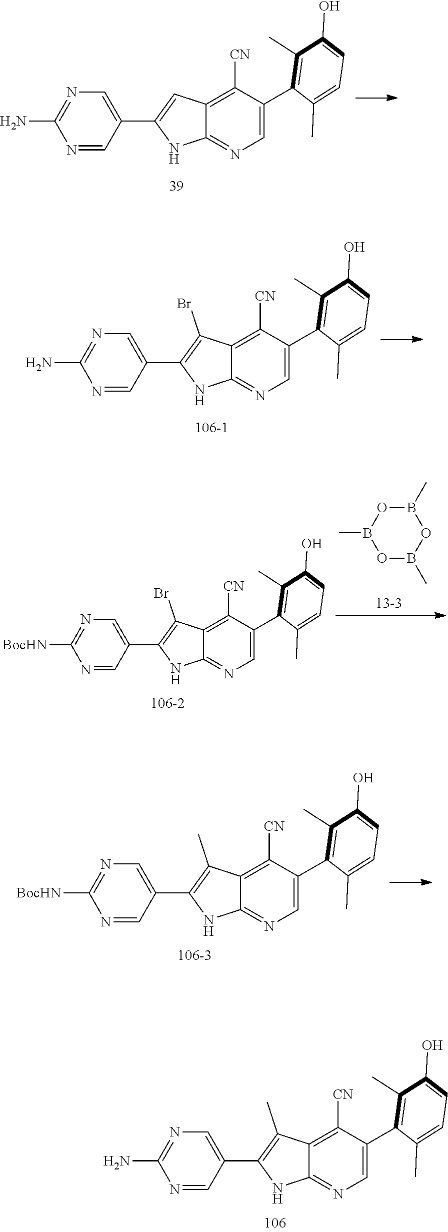

### Step 1: Preparation of Compound 106-1

To a solution of compound 39 (100.0 mg, 0.281 mmol) in CH₃CN (5 mL) was added NBS (44.9 mg, 0.252 mmol) at 0° C. The mixture was stirred at rt for 16 h. The reaction mixture was diluted with water (30 mL) and extracted with EA (30 mL×3). The combined organic layer was washed with brine (50 mL), dried over Na₂SO₄, filtered, and concentrated to dryness. The residue was purified by silica gel column chromatography to afford compound 106-1 (120.0 mg, 0.276 mmol, 98.2%). LCMS: 435.2 [M+H]⁺.

### Step 2: Preparation of Compound 106-2

To a solution of compound 106-1 (120.0 mg, 0.276 mmol) in DMF (6 mL) were added (Boc)₂O (60.2 mg, 0.276 mmol), DMAP (1.7 mg, 0.014 mmol) and TEA (41.8 mg, 0.1414 mmol). The mixture was stirred at rt for 2 h. The reaction mixture was diluted with water (30 mL) and extracted with EA (30 mL×3). The combined organic layer was washed with brine (50 mL×3), dried over Na₂SO₄, filtered, and concentrated to dryness. The residue was purified by silica gel column chromatography to afford compound 106-2. LCMS: 535.2 [M+H]⁺.

### Step 3: Preparation of Compound 106-3

To a solution of compound 106-2 (70.0 mg, 0.131 mmol) in H₂O (0.5 mL) and dioxane (2.5 mL) were added compound 13-3 (32.9 mg, 0.262 mmol), K₃PO₄ (83.4 mg, 0.393 mmol) and Pd(dtbpf)Cl₂ (8.6 mg, 0.0132 mmol). The mixture was stirred at 80° C. for 6 h. The reaction mixture was diluted with water (30 mL) and extracted with EA (30 mL×3). The combined organic layer was washed with brine (50 mL), dried over Na₂SO₄, filtered, and concentrated to dryness. The residue was purified by silica gel column chromatography to afford compound 106-3. LCMS: 471.4 [M+H]⁺.

### Step 4: Preparation of Example 106

A mixture of compound 106-3 (43.0 mg, 0.0914 mmol) in HCl-dioxane (2.5 mL) was stirred at rt for 1 h. The reaction mixture was concentrated to dryness and purified by prep-HPLC to afford example 106. LCMS: 371.2 [M+H]⁺; ¹H NMR (400 MHz, DMSO-d₆) δ 12.42 (s, 1H), 9.33 (s, 1H), 8.59 (s, 2H), 8.06 (s, 1H), 7.09 (s, 2H), 7.00 (d, J=8.4 Hz, 1H), 6.83 (d, J=8.4 Hz, 1H), 2.53 (s, 3H), 1.86 (s, 3H), 1.79 (s, 3H).

The following examples 107-108 were prepared according to the methods described in example 106 using analogous starting materials.

| | | LCMS | |
|---|---|---|---|
| Example | Structure | [M + H]⁺ | ¹H NMR |
| 107 | 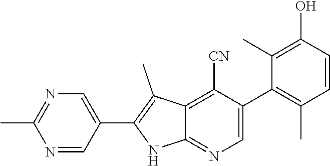 | 411.0 [M + H + CH₃CN]⁺ | (400 MHz, CD₃OD) δ 9.03 (s, 2H), 8.14 (s, 1H), 7.02 (d, J = 8.4 Hz, 1H), 6.82 (d, J = 8.4 Hz, 1H), 2.80 (s, 3H), 2.67 (s, 3H), 1.93 (s, 3H), 1.89 (s, 3H). |
| | 5-(3-hydroxy-2,6- | | |
| | dimethylphenyl)-3-methyl- | | |
| | 2-(2-methylpyrimidin-5- | | |
| | yl)-1H-pyrrolo[2,3- | | |
| | b]pyridine-4-carbonitrile | | |
| 108 | 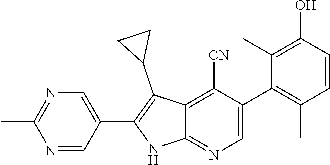 [SMILES: Cc1ncc(-c2[nH]c3ncc(-c4c(C)ccc(O)c4C)c(C#N)c3c2C2CC2)cn1] | 396.1 | (400 MHz, DMSO-d₆) δ 12.72 (s, 1H), 9.34 (s, 1H), 9.17 (s, 2H), 8.18 (s, 1H), 7.01 (d, J = 8.4 Hz, 1H), 6.85 (d, J = 8.4 Hz, 1H), 2.73 (s, 3H), 2.28-2.16 (m, 1H), 1.87 (s, 3H), 1.80 (s, 3H), 0.99-0.94 (m, 2H), 0.35-0.31 (m, 2H). |
| | 3-cyclopropyl-5-(3- | | |
| | hydroxy-2,6- | | |
| | dimethylphenyl)-2-(2- | | |
| | methylpyrimidin-5-yl)-1H- | | |
| | pyrrolo[2,3-b]pyridine-4- | | |
| | carbonitrile | | |

### Example 109: Preparation of (S)-3-ethynyl-5-(3-hydroxy-2,6-dimethylphenyl)-2-(2-methylpyrimidin-5-yl)-1H-pyrrolo[2,3-b]pyridine-4-carbonitrile

The title compound was prepared according to the following synthetic procedures.

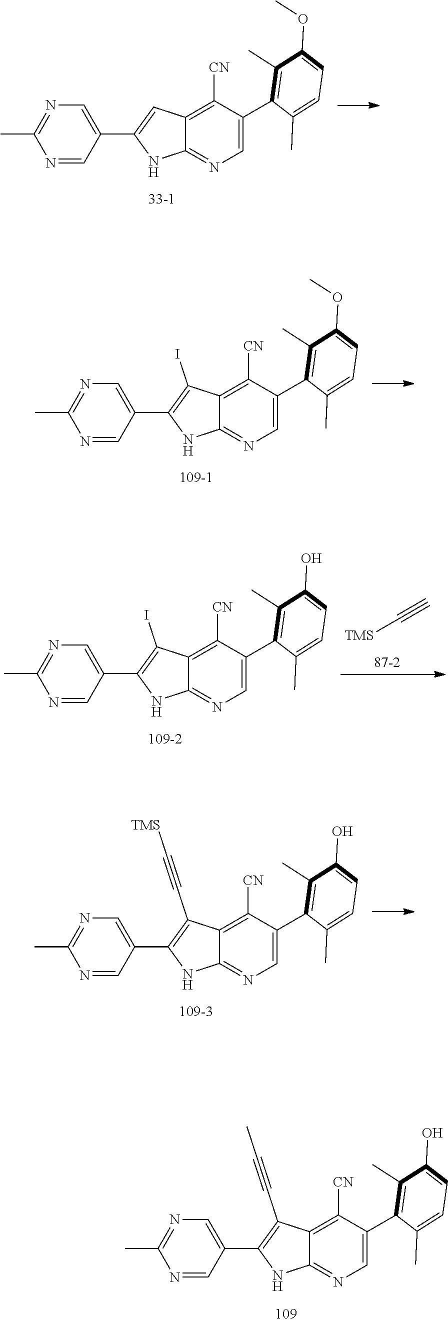

### Step 1: Preparation of Compound 109-1

To a solution of compound 33-1 (141 mg, 0.382 mmol) in CAN/DMF (1:1, 5 mL) was added NIS (94.5 mg, 0.420 mmol) at 0 [symbol] . The mixture was stirred at room temperature for 2 h. The reaction mixture was diluted with EA (60 mL), which was washed with brine (30 mL×3). The organic layer was dried over Na₂SO₄, filtered, and concentrated to dryness. The residue was purified by silica gel column chromatography to afford compound 109-1. LCMS: 496.4 [M+H]⁺.

### Step 2: Preparation of Compound 109-2

To a solution of compound 109-1 (85 mg, 0.172 mmol) in DCM (5 mL) was added BBr₃ (215.5 mg, 0.860 mmol). The mixture was stirred at rt for 1 h under N₂ atmosphere. The mixture was quenched by MeOH (5 mL) and concentrated. The residue was purified by column chromatography on silica gel to afford compound 109-2. LCMS: 482.1[M+H]⁺.

### Step 3: Preparation of Compound 109-3

To a solution of compound 109-2 (100.0 mg, 0.208 mmol) in DMF (3 mL) were added compound 87-2 (61.2 mg, 0.623 mmol), CuI (7.9 mg, 0.042 mmol), PdCl₂(PPh₃)₂ (16.2 mg, 0.021 mmol) and TEA (210.3 mg, 2.078 mmol). The mixture was stirred at 80° C. for 2 h. The mixture was purified by prep-HPLC to afford compound 109-3. LCMS: 452.5 [M+H]⁺.

### Step 4: Preparation of Example 109

To a solution of compound 109-3 (20.0 mg, 0.0443 mmol) in MeOH (3 mL) was added K₂CO₃ (18.4 mg, 0.133 mmol). The mixture was stirred at rt for 16 h. The mixture was purified by prep-HPLC to afford example 109. LCMS: 380.1 [M+H]⁺; ¹H NMR (400 MHz, CD₃OD) δ 9.44 (s, 2H), 8.24 (s, 1H), 7.02 (d, J=8.4 Hz, 1H), 6.83 (d, J=8.4 Hz, 1H), 4.00 (s, 1H), 2.80 (s, 3H), 1.94 (s, 3H), 1.89 (s, 3H).

The following examples 110-111 were prepared according to the methods described in example 109 using analogous starting materials.

| | | LCMS | |
|---|---|---|---|
| Example | Structure | [M + H]⁺ | ¹H NMR |
| 110 | 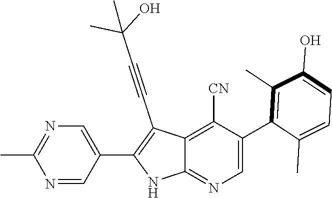 | 438.0 | (400 MHz, CD₃OD) δ 9.47 (s, 2H), 8.22 (s, 1H), 7.02 (d, J = 8.4 Hz, 1H), 6.83 (d, J = 8.4 Hz, 1H), 2.80 (s, 3H), 1.93 (s, 3H), 1.89 (s, 3H), 1.62 (s, 6H). |
| | (S)-5-(3-hydroxy-2,6- | | |
| | dimethylphenyl)-3-(3- | | |
| | hydroxy-3-methylbut-1-yn-1- | | |
| | yl)-2-(2-methylpyrimidin-5- | | |
| | yl)-1H-pyrrolo[2,3- | | |
| | b]pyridine-4-carbonitrile | | |
| 111 | 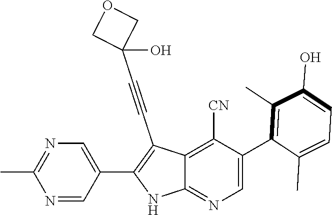 | 452.2 | (400 MHz, DMSO-d₆) δ 13.45 (s, 1H), 9.42 (s, 2H), 9.37 (s, 1H), 8.31 (s, 1H), 7.03 (d, J = 8.4 Hz, 1H), 6.86 (d, J = 8.4 Hz, 1H), 6.64 (s, 1H), 4.85 (d, J = 6.2 Hz, 2H), 4.58 (d, J = 6.2 Hz, 2H), 2.74 (s, 3H), 1.88 (s, 3H), 1.80 (s, 3H). |
| | (S)-5-(3-hydroxy-2,6- | | |
| | dimethylphenyl)-3-((3- | | |
| | hydroxyoxetan-3-yl)ethynyl)- | | |
| | 2-(2-methylpyrimidin-5-yl)- | | |
| | 1H-pyrrolo[2,3-b]pyridine-4- | | |
| | carbonitrile | | |

### Example 112: Preparation of 5-(3-hydroxy-2,6-dimethylphenyl)-2-(2-methylpyrimidin-5-yl)-3-(trifluoromethyl)-1H-pyrrolo[2,3-b]pyridine-4-carbonitrile

The title compound was prepared according to the following synthetic procedures.

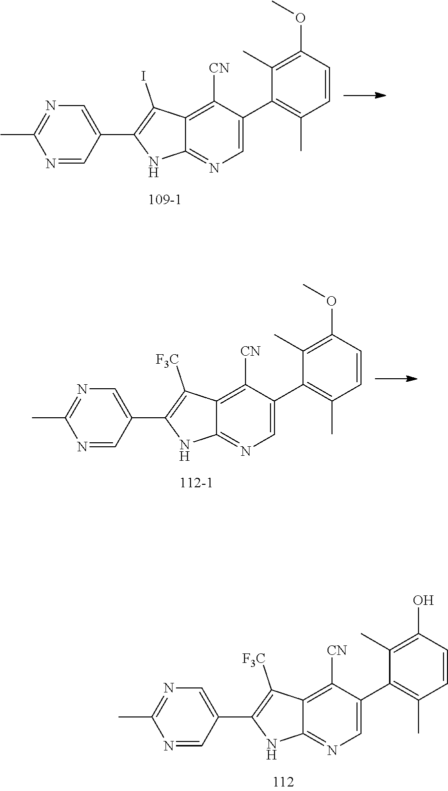 [SMILES: COc1ccc(C)c(-c2cnc3[nH]c(-c4cnc(C)nc4)c(I)c3c2C#N)c1C ; COc1ccc(C)c(-c2cnc3[nH]c(-c4cnc(C)nc4)c(C(F)(F)F)c3c2C#N)c1C ; Cc1ncc(-c2[nH]c3ncc(-c4c(C)ccc(O)c4C)c(C#N)c3c2C(F)(F)F)cn1]

### Step 1: Preparation of Compound 112-1

To a solution of compound 109-1 (50 mg, 0.101 mmol) in DMF (2 mL) were added Cu (12.84 mg, 0.202 mmol) and diphenyl(trifluoromethyl)sulfonium (204.20 mg, 0.505 mmol) at rt. The mixture was stirred at 60° C. for 24 h. The mixture was quenched with H₂O (5 mL) and extracted with EA (20 mL). The organic layer was dried over Na₂SO₄ and concentrated to dryness. The residue was purified by silica gel column chromatography to afford compound 112-1. LCMS: 438.4 [M+H]⁺.

### Step 2: Preparation of Example 112

To a solution of compound 112-1 (22 mg, 0.050 mmol) in DCM (5 mL) was added BBr₃ (0.12 mL, 0.251 mmol). The mixture was stirred at room temperature under N₂ for 0.5 h. The reaction was quenched with MeOH (5 mL). The mixture was concentrated to dryness. The residue was purified by prep-HPLC to afford example 112. LCMS: 424.1 [M+H]⁺; ¹H NMR: (400 MHz, DMSO-d₆) δ 9.40 (s, 1H), 9.00 (s, 2H), 8.39 (s, 1H), 7.02 (d, J=8.4 Hz, 1H), 6.85 (d, J=8.4 Hz, 1H), 2.74 (s, 3H), 1.85 (s, 3H), 1.78 (s, 3H).

### Example 113: Preparation of 5-(3-hydroxy-2,6-dimethylphenyl)-2-(2-methylpyrimidin-5-yl)-3-(phenylthio)-1H-pyrrolo[2,3-b]pyridine-4-carbonitrile

The title compound was prepared according to the following synthetic procedures.

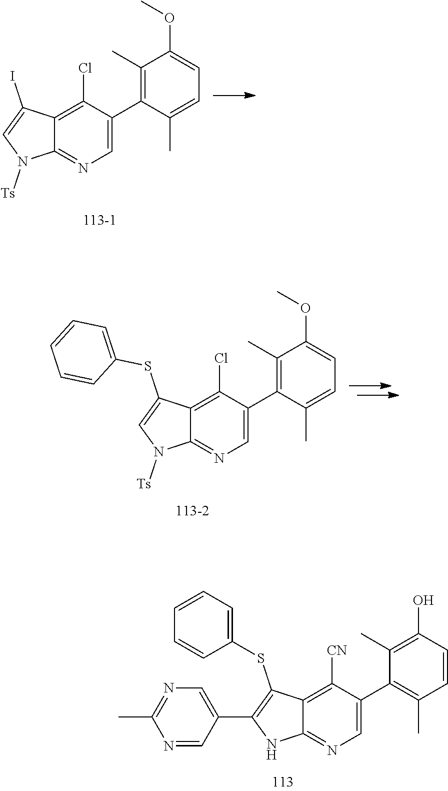 [SMILES: COc1ccc(C)c(-c2cnc3c(c(I)cn3S(=O)(=O)c3ccc(C)cc3)c2Cl)c1C ; COc1ccc(C)c(-c2cnc3c(c(Sc4ccccc4)cn3S(=O)(=O)c3ccc(C)cc3)c2Cl)c1C ; Cc1ncc(-c2[nH]c3ncc(-c4c(C)ccc(O)c4C)c(C#N)c3c2Sc2ccccc2)cn1]

### Step 1: Preparation of Compound 113-2

To a solution of compound 113-1 (200 mg, 0.353 mmol) in DMF (10 mL) were added sodium thiophenoxide (69.8 mg, 0.529 mmol), CuI (67.1 mg, 0.353 mmol), ethylene glycol (43.8 mg, 0.706 mmol) and K₂CO₃ (146.3 mg, 1.059 mmol). The mixture was stirred at 80° C. for 3 h. The reaction mixture was filtered. The filtrate was concentrated to dryness. The residue was purified by silica gel column chromatography to afford compound 113-2 (170 mg, 0.310 mmol, 87.75%) as a yellow solid. LCMS: 549.1 [M+H]⁺.

### Step 2: Preparation of Example 113

The example 113 was prepared from compound 113-2 according to the methods described in example 32-33. Spectrum data of example 113: LCMS: 464.2 [M+H]⁺; ¹H NMR: (400 MHz, CD₃OD) δ 9.15 (s, 2H), 8.26 (s, 1H), 7.21 (t, J=7.6 Hz, 2H), 7.11 (d, J=7.3 Hz, 1H), 7.03 (d, J=7.5 Hz, 2H), 6.97 (d, J=8.2 Hz, 1H), 6.78 (d, J=8.2 Hz, 1H), 2.75 (s, 3H), 1.86 (s, 3H), 1.81 (s, 3H).

### Example 114: Preparation of 2,4-dimethyl-3-(2-(2-methylpyrimidin-5-yl)-3H-imidazo[4,5-b]pyridin-6-yl)phenol

The title compound was prepared according to the following synthetic procedures.

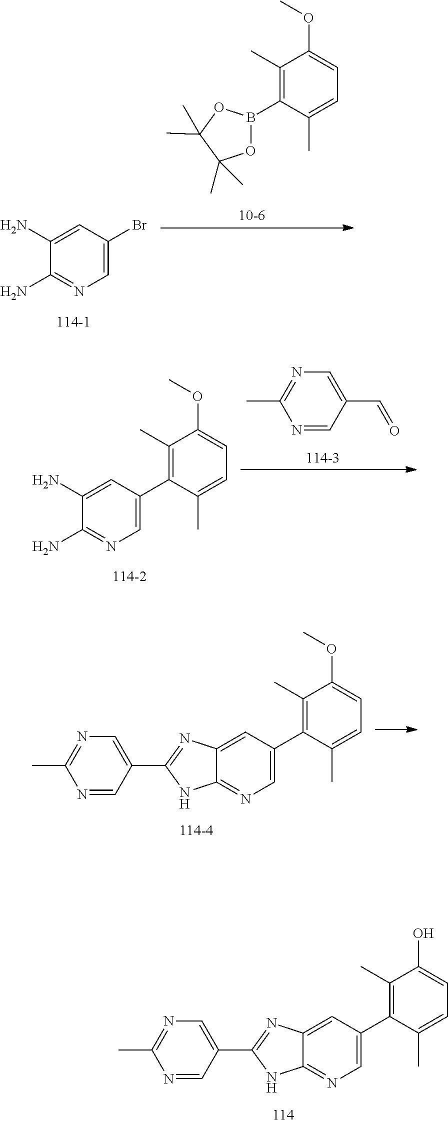

### Step 1: Preparation of Compound 114-2

To a solution of compound 114-1 (500 mg, 2.659 mmol) in dioxane (5 mL) and water (1 mL) were added compound 10-6 (836.5 mg, 3.191 mmol), Pd(dppf)Cl₂ (194.6 mg, 0.266 mmol) and K₂CO₃ (1.102 g, 7.977 mmol). The mixture was stirred at 90° C. under N₂ for 2 h. The reaction mixture was diluted with water (20 mL) and extracted with EA (30 mL×3). The combined organic layer was washed with brine (30 mL), dried over Na₂SO₄, filtered, and concentrated to dryness. The residue was purified by silica gel column chromatography to afford compound 114-2. LCMS: 244.2 [M+H]⁺.

### Step 2: Preparation of Compound 114-4

To a solution of compound 114-2 (150 mg, 0.616 mmol) in AcOH (5 mL) was added compound 114-3 (75.29 mg, 0.616 mmol). The mixture was stirred at 90° C. under N₂ for 1 h. The reaction mixture was diluted with water (20 mL) and extracted with EA (30 mL×3). The combined organic layer was washed with brine (30 mL), dried over Na₂SO₄, filtered, and concentrated to dryness. The residue was purified by silica gel column chromatography to afford compound 114-4. LCMS: 346.0 [M+H]⁺.

### Step 3: Preparation of Example 114

To a solution of compound 114-4 (160 mg, 0.463 mmol) in DCM (3 mL) was added BBr₃ (1.16 g, 4.63 mmol). The mixture was stirred at rt under N₂ for 2 h. The reaction mixture was diluted with water (15 mL) and extracted with EA (20 mL×3). The combined organic layer was washed with brine (20 mL), dried over Na₂SO₄, filtered, and concentrated to dryness. The residue was purified by prep-TLC to afford example 114. LCMS: 332.1 [M+H]⁺; ¹H NMR: (400 MHz, DMSO-d₆) δ 9.45 (s, 2H), 8.19 (s, 1H), 7.94 (d, J=1.4 Hz, 1H), 6.95 (s, 1H), 6.79 (d, J=8.2 Hz, 1H), 2.74 (s, 3H), 1.89 (s, 3H), 1.82 (s, 3H).

### Example 115: Preparation of 3-(2-(2-(cyclopropylamino)thiazol-5-yl)-3H-imidazo[4,5-b]pyridin-6-yl)-2,4-dimethylphenol

The title compound was prepared according to the following synthetic procedures.

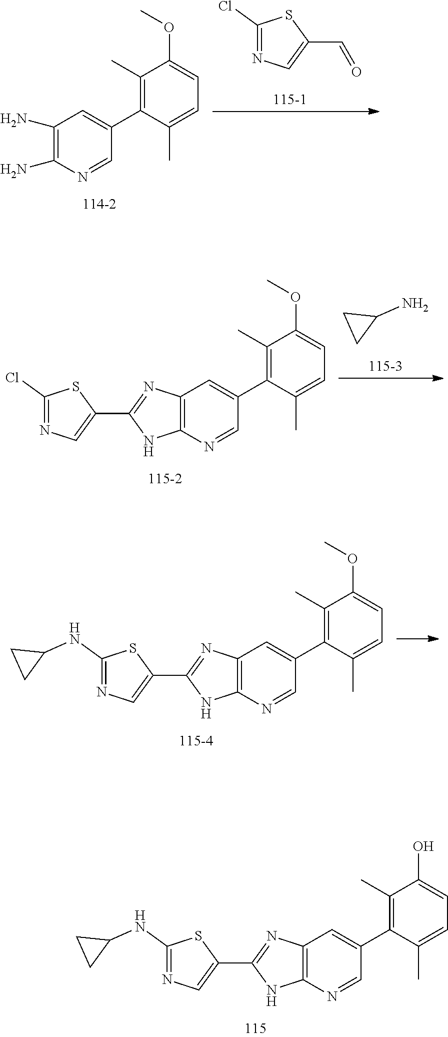

### Step 1: Preparation of Compound 115-2

To a solution of compound 114-2 (100 mg, 0.41 mmol) in AcOH (1.5 mL) was added compound 115-1 (73 mg, 0.49 mmol). The mixture was stirred at 90° C. under N₂ for 20 h. The reaction was diluted with EA (10 ml) and brine (10 ml). The aqueous layer was extracted with EA (10 mL×3). The combined organic layer was washed with brine, dried over Na₂SO₄, filtered and concentrated. The residue was purified by prep-TCL to afford compound 115-2. LCMS: 371.0 [M+H]⁺.

### Step 2: Preparation of Compound 115-4

To a solution of compound 115-2 (40 mg, 0.11 mmol) in CH₃CN (1.5 mL) was added compound 115-3 (1 mL). The mixture was stirred at 100° C. for 3 h. The reaction was dried to afford the title crude compound 115-4. LCMS: 392.1 [M+H]⁺.

### Step 3: Preparation of Example 115

To solution of compound 115-4 (55 mg, 0.14 mmol) in DCM (2 mL) was added BBr₃ (0.1 mL), and the reaction was stirred at rt for 0.5 h under N₂. The reaction was quenched with MeOH (3 mL) and NH₃/MeOH (3 mL). The residue was purified by prep-HPLC to afford example 115. LCMS: 378.1[M+H]⁺; ¹H NMR: (400 MHz, DMSO-d₆) δ 13.33 (s, 1H), 9.18 (s, 1H), 8.57 (s, 1H), 7.98 (s, 1H), 7.92 (s, 1H), 7.61 (s, 1H), 6.93 (d, J=8.2 Hz, 1H), 6.76 (d, J=8.2 Hz, 1H), 2.63-2.59 (m, 1H), 1.88 (s, 3H), 1.81 (s, 3H), 0.93-0.76 (m, 2H), 0.62-0.57 (m, 2H).

### Example 116: Preparation of (S)-2-(2-aminopyrimidin-5-yl)-6-(3-hydroxy-2,6-dimethylphenyl)-3H-imidazo[4,5-b]pyridine-7-carbonitrile

The title compound was prepared according to the following synthetic procedures.

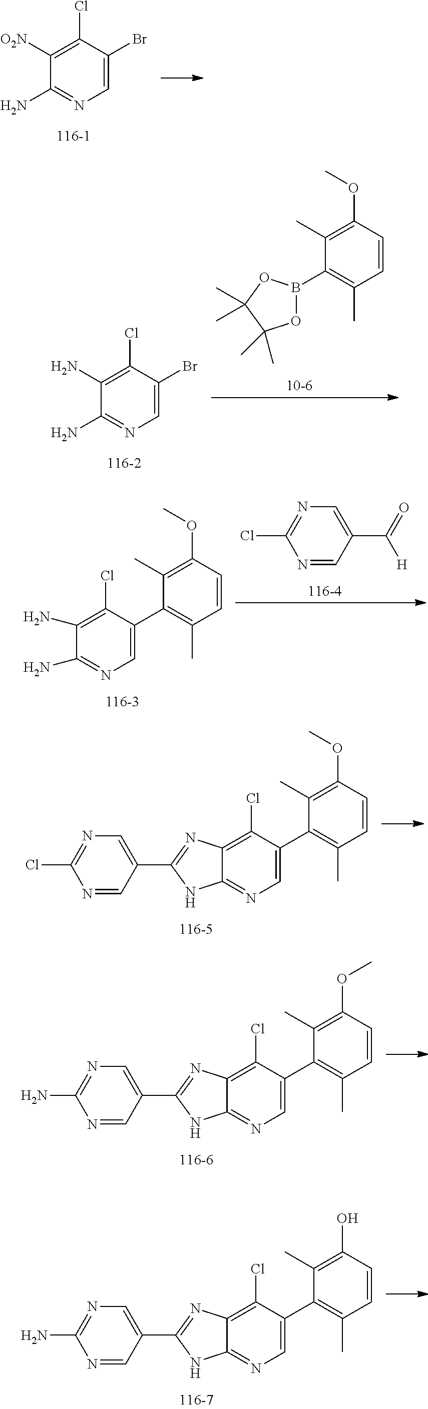

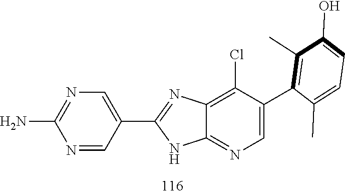

### Step 1: Preparation of Compound 116-2

To a solution of compound 116-1 (5 g, 19.802 mmol) in EtOH (150 mL) was added Fe (11.0 g, 198.020 mmol) and AcOH (22.67 mL, 396.040 mmol). The mixture was stirred at 90° C. for 2 h. The reaction mixture was carefully added into ice-water (150 mL) while stirring, the pH of which was adjusted to 8 with NaOH (4 M in water). The mixture was extracted with DCM (200 mL×3). The combined organic layers were washed with brine (300 mL), dried over Na₂SO₄, filtered, and concentrated to dryness. The residue was purified by silica gel column chromatography to afford compound 116-2 (3.5 g, 15.730 mmol, 79.4%). LCMS: 221.9 [M+H]⁺.

### Step 2: Preparation of Compound 116-3

To a solution of compound 116-2 (900 mg, 4.045 mmol) in dioxane (22.5 mL) and H₂O (4.5 mL) were added compound 10-6 (3.182 g, 12.135 mmol), Pd(dtbpf)Cl₂ (264.1 mg, 0.404 mmol) and K₂CO₃ (1.677 g, 12.135 mmol). The mixture was stirred at 100° C. for 16 h. The reaction mixture was diluted with water (50 mL), which was extracted with EA (40 mL×3). The combined organic layers were washed with brine (100 mL), dried over Na₂SO₄, filtered, and concentrated to dryness. The residue was purified by silica gel column chromatography to afford compound 116-3 (240 mg, 0.864 mmol, 21.4%). LCMS: 278.2 [M+H]⁺.

### Step 3: Preparation of Compound 116-5

To a solution of compound 116-3 (200 mg, 0.720 mmol) in dioxane (4 mL) was added compound 116-4 (102.6 mg, 0.720 mmol) and AcOH (2 mL). The mixture was stirred at 75° C. for 8 h. The reaction mixture was carefully added into ice-water (20 mL) while stirring, the pH of which was adjusted to 8 with NaHCO₃ (aq. sat). The mixture was extracted with EA (20 mL×3). The combined organic layers were washed with brine (30 mL), dried over Na₂SO₄, filtered, and concentrated to dryness. The residue was purified by silica gel column chromatography to afford compound 116-5 (67 mg, 0.167 mmol, 23.2%). LCMS: 400.1 [M+H]⁺.

### Step 4: Preparation of Compound 116-6

A solution of compound 116-5 (60 mg, 0.150 mmol) in NH₃/EtOH (1.5 mL) was stirred at 70 [symbol] for 48 h. The reaction mixture was diluted with water (10 mL), which was extracted with EA (10 mL×3). The combined organic layer was washed with brine (20 mL), dried over Na₂SO₄, filtered, and concentrated to dryness. The residue was purified by silica gel column chromatography to afford compound 116-6. LCMS: 381.2 [M+H]⁺.

### Step 5: Preparation of Compound 116-7

To a solution of compound 116-6 (45 mg, 0.118 mmol) in DCM (1 mL) was added BBr₃ (0.4 mL). The mixture was stirred at rt under N₂ for 2 h. The reaction mixture was carefully added into MeOH (10 mL). Then the pH of the mixture was adjusted to 7-8 with TEA. After filtration, the filtrate was concentrated to dryness. The residue was purified by silica gel column chromatography to afford compound 116-7. LCMS: 367.1 [M+H]⁺.

### Step 6: Preparation of Example 116

To a solution of compound 116-7 (21 mg, 0.057 mmol) in DMA (1 mL) were added Zn (3.7 mg, 0.057 mmol), Zn (CN)₂ (6.7 mg, 0.057 mmol) and Pd(dppf)Cl₂·CH₂Cl₂ (46.8 mg, 0.057 mmol), Pd₂(dba)₃ (52.4 mg, 0.057 mmol). The mixture was stirred at 140° C. under N₂ for 18 h. The residue was purified by prep-HPLC and further purified by SFC to afford example 116. LCMS: 358.3 [M+H]⁺; ¹H NMR: (400 MHz, CD₃OD) δ 9.09 (s, 2H), 8.19 (s, 1H), 7.02 (d, J=8.2 Hz, 1H), 6.83 (d, J=8.2 Hz, 1H), 1.94 (s, 3H), 1.89 (s, 3H).

### Example 117: Preparation of (S)-5-(3-hydroxy-2,6-dimethylphenyl)-2-(1-(2-hydroxy-2-methylpropyl)-1H-pyrazol-4-yl)-1H-pyrrolo[2,3-b]pyridine-4-carbonitrile

The title compound was prepared according to the following synthetic procedures.

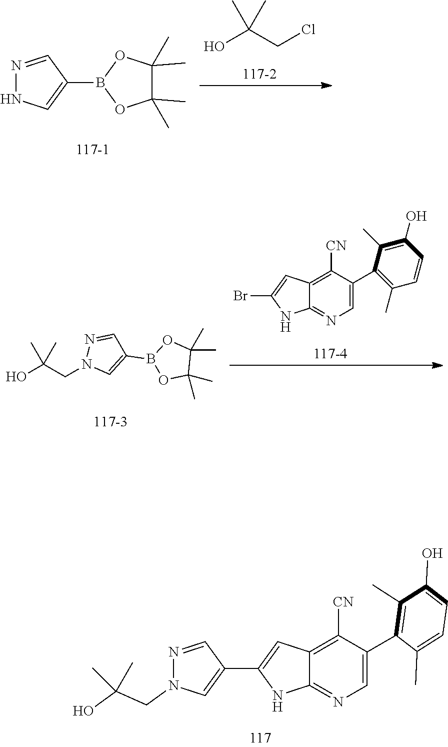

### Step 1: Preparation of Compound 117-3

To a solution of compound 117-1 (1 g, 5.155 mmol) and compound 117-2 (1.2 g, 10.825 mmol) in DMF (10 mL) was added Cs₂CO₃ (4.2 g, 12.887 mmol). The mixture was stirred at 140° C. for 2.5 h by microwave. The reaction mixture was diluted with EA (20 mL), which was washed with brine (20 mL×3). The organic layers were dried over Na₂SO₄, filtered, and concentrated to dryness. The residue was purified by silica gel column chromatography to afford compound 117-3. LCMS: 266.6 [M+H]⁺.

### Step 2: Preparation of Example 117

To a solution of compound 117-3 (40 mg, 0.117 mmol) and compound 117-4 (62 mg, 0.234 mmol) in H₂O (0.2 mL) and dioxane (1 mL) was added K₂CO₃ (49 mg, 0.351 mmol) and Pd(dtbpf)Cl₂ (8.0 mg, 0.012 mmol). The reaction mixture was diluted with water (10 mL), which was extracted with EA (10 mL×3). The combined organic layers were washed with brine (20 mL), dried over Na₂SO₄, filtered, and concentrated to dryness. The residue was purified by prep-TLC to afford example 117. LCMS: 402.2 [M+H]⁺; ¹H NMR: (400 MHz, DMSO-d₆) δ 12.65 (s, 1H), 9.32 (s, 1H), 8.38 (s, 1H), 8.15 (s, 1H), 8.00 (s, 1H), 7.00 (d, J=8.2 Hz, 1H), 6.85 (dd, J=16.3, 4.9 Hz, 2H), 4.81 (s, 1H), 4.09 (s, 2H), 1.87 (s, 3H), 1.79 (s, 3H), 1.11 (s, 6H).

The following examples 118-122 were prepared according to the methods described in examples 117 with analogous starting materials.

| | | LCMS | |
|---|---|---|---|
| Example | Structure | [M + H]⁺ | ¹H NMR |
| 118 | 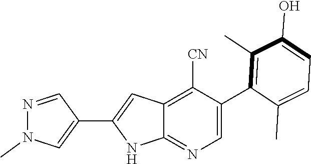 | 344.1 | (400 MHz, DMSO-d₆) δ 12.64 (s, 1H), 9.32 (s, 1H), 8.36 (s, 1H), 8.13 (s, 1H), 8.01 (s, 1H), 6.99 (d, J = 8.1 Hz, 1H), 6.83 (d, J = 7.5 Hz, 2H), 3.93 (s, 3H), 1.87 (s, 3H), 1.79 (s, 3H). |
| | (S)-5-(3-hydroxy-2,6- | | |
| | dimethylphenyl)-2-(1-methyl-1H- | | |
| | pyrazol-4-yl)-1H-pyrrolo[2,3- | | |
| | b]pyridine-4-carbonitrile | | |
| 119 | 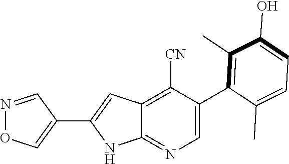 | 331.1 | (400 MHz, DMSO-d₆) δ 12.92 (s, 1H), 9.56 (s, 1H), 9.36 (s, 2H), 8.15 (s, 1H), 7.14 (s, 1H), 7.01 (d, J = 6.6 Hz, 1H), 6.85 (d, J = 8.0 Hz, 1H), 1.87 (s, 3H), 1.79 (s, 3H). |
| | (S)-5-(3-hydroxy-2,6- | | |
| | dimethylphenyl)-2-(isoxazol-4-yl)- | | |
| | 1H-pyrrolo[2,3-b]pyridine-4- | | |
| | carbonitrile | | |
| 120 | 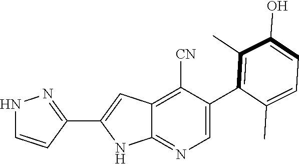 | 329.9 | (400 MHz, DMSO-d₆) δ 13.27 (s, 1H), 12.81 (s, 1H), 9.33 (s, 1H), 8.09 (s, 1H), 7.90 (s, 1H), 7.03- 6.98 (m, 2H), 6.95 (s, 1H), 6.84 (d, J = 8.2 Hz, 1H), 1.87 (s, 3H), 1.79 (s, 3H). |
| | (S)-5-(3-hydroxy-2,6- | | |
| | dimethylphenyl)-2-(1H-pyrazol-3- | | |
| | yl)-1H-pyrrolo[2,3-b]pyridine-4- | | |
| | carbonitrile | | |

### Example 121: Preparation of (S)-5-(3-hydroxy-2,6-dimethylphenyl)-2-(1H-1,2,4-triazol-1-yl)-1H-pyrrolo[2,3-b]pyridine-4-carbonitrile

The title compound was prepared according to the following synthetic procedures.

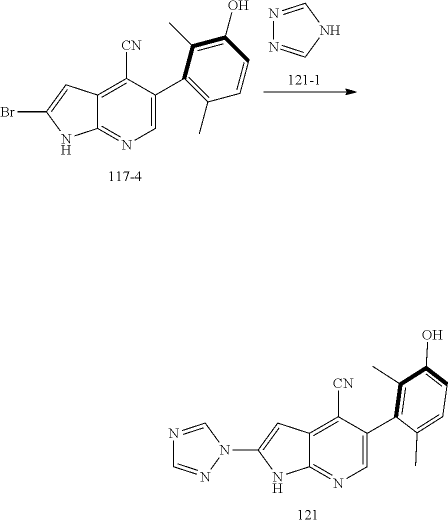

### Step 1: Preparation of Example 121

A mixture of compound 117-4 (50 mg, 0.146 mmol) and compound 121-1 (101.0 mg, 1.461 mmol) was stirred at 140° C. for 18 h. The reaction mixture was diluted with water (10 mL), which was extracted with EA (10 mL×3). The combined organic layers were washed with brine (20 mL), dried over Na₂SO₄, filtered, and concentrated to dryness. The residue was purified by silica gel column chromatography to afford example 121. LCMS: 331.1 [M+H]⁺; ¹H NMR: (400 MHz, DMSO-d₆) δ 13.62 (s, 1H), 9.44 (s, 1H), 9.37 (s, 1H), 8.43 (s, 1H), 8.21 (s, 1H), 7.07 (s, 1H), 7.02 (d, J=8.2 Hz, 1H), 6.85 (d, J=8.2 Hz, 1H), 1.87 (s, 3H), 1.80 (s, 3H).

### Example 122: Preparation of (S)-5-(3-hydroxy-2,6-dimethylphenyl)-2-(1H-imidazol-1-yl)-1H-pyrrolo[2,3-b]pyridine-4-carbonitrile

The title compound was prepared according to the following synthetic procedures.

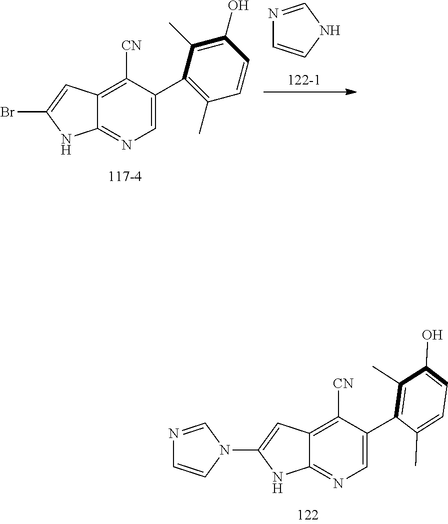

### Step 1: Preparation of Example 122

A mixture of compound 117-4 (40 mg, 0.117 mmol) and compound 122-1 (119.4 mg, 1.753 mmol) was heated to 140 [symbol] for 16 h. The mixture was purified by prep-TLC to afford example 122. LCMS: 330.2 [M+H]⁺; ¹H NMR: (400 MHz, DMSO-d₆) δ 13.37 (s, 1H), 9.36 (s, 1H), 8.56 (s, 1H), 8.14 (s, 1H), 8.01 (s, 1H), 7.23 (s, 1H), 7.01 (d, J=6.4 Hz, 2H), 6.85 (d, J=8.2 Hz, 1H), 1.87 (s, 3H), 1.79 (s, 3H).

### Example 123: Preparation of (S)-5-(3-hydroxy-2,6-dimethylphenyl)-2-(1H-pyrazolo[3,4-b]pyridin-5-yl)-1H-pyrrolo[2,3-b]pyridine-4-carbonitrile

The title compound was prepared according to the following synthetic procedures.

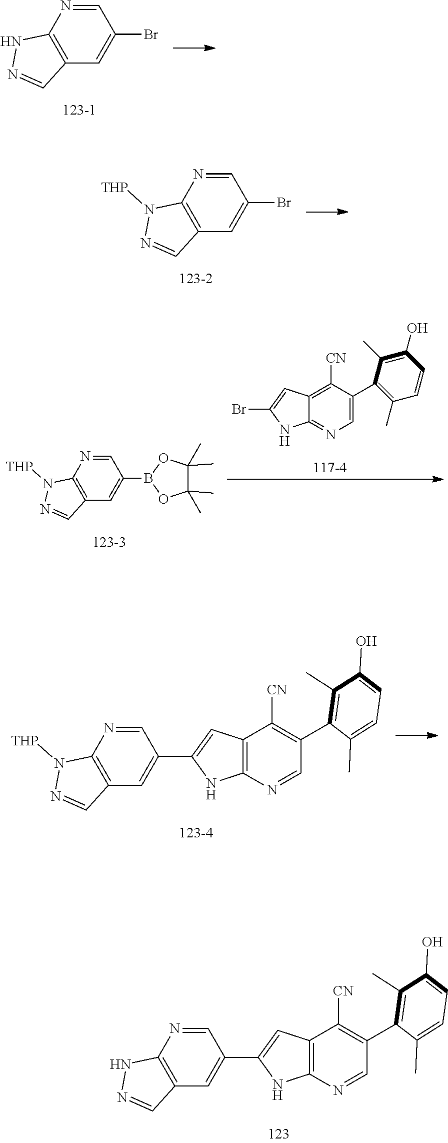

### Step 1: Preparation of Compound 123-2

A mixture of compound 123-1 (1000 mg, 5.051 mmol), 3,4-dihydro-2H-pyran (1274 mg, 15.152 mmol), and PPTS (127 mg, 0.505 mmol) in CH₃CN (8 mL) and DCM (8 mL) was stirred at 50° C. for 16 h. The mixture was concentrated. The residue was purified by silica gel column chromatography to afford compound 123-2 (1200 mg, 4.254 mmol, 84.2%) as a yellow solid. LCMS: 282.1 [M+H]⁺.

### Step 2: Preparation of Compound 123-3

To a solution of compound 123-2 (150 mg, 0.532 mmol) in dioxane (2 mL) was added 4,4,5,5-tetramethyl-2-(4,4,5,5-tetramethyl-1,3,2-dioxaborolan-2-yl)-1,3,2-dioxaborolane (270 mg, 1.063 mmol), KOAc (130 mg, 1.329 mmol) and Pd(dppf)Cl₂ (39 mg, 0.053 mmol). The mixture was stirred at 90° C. for 16 h. The reactants proceed directly to the next step. LCMS: 330.2 [M+H]⁺.

### Step 3: Preparation of Compound 123-4

To a solution of compound 123-3 (87 mg, 0.263 mmol) and compound 117-4 (50 mg, 0.146 mmol) in dioxane (2 mL) and water (0.4 mL) was added K₂CO₃ (60 mg, 0.438 mmol) and Pd(dtbpf)Cl₂ (10 mg, 0.015 mmol). The mixture was stirred at 100° C. for 3 h. The mixture was concentrated. The residue was purified by prep-TLC to afford compound 123-4. LCMS: 465.4 [M+H]⁺.

### Step 4: Preparation of Example 123

A mixture of compound 123-4 (20 mg, 0.043 mmol) and hydrogen chloride (4 mL, 16.000 mmol) in MeOH (4 mL) was stirred at rt for 4 h. The mixture was concentrated. The crude was diluted with water (10 mL), which was adjusted to pH˜8 with Na₂CO₃(aq). The mixture was extracted with EA (40 mL). The combined organic layer was washed with brine (20 mL), dried over Na₂SO₄, filtered, and concentrated to dryness. The residue was purified by prep-TLC to afford example 123. LCMS: 381.1 [M+H]⁺; ¹H NMR: (400 MHz, DMSO-d₆) δ 13.87 (s, 1H), 12.98 (s, 1H), 9.33 (s, 1H), 9.25 (d, J=2.0 Hz, 1H), 8.89 (d, J=1.8 Hz, 1H), 8.29 (s, 1H), 8.14 (s, 1H), 7.31 (d, J=1.9 Hz, 1H), 7.01 (d, J=8.1 Hz, 1H), 6.85 (d, J=8.1 Hz, 1H), 1.89 (s, 3H), 1.81 (s, 3H).

The following examples were prepared according to the methods described in examples 123 with analogous starting materials.

| | | LCMS | |
|---|---|---|---|
| Example | Structure | [M + H]⁺ | ¹H NMR |
| 124 | 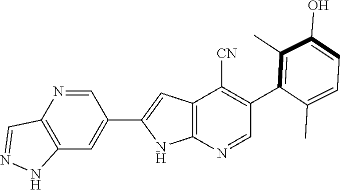 | 421.9 [M + H + CH₃CN]⁺ | (400 MHz, DMSO-d₆) δ 13.65 (s, 1H), 13.09 (s, 1H), 9.34 (s, 1H), 9.26 (d, J = 1.8 Hz, 1H), 8.65 (s, 1H), 8.37 (s, 1H), 8.19 (s, 1H), 7.47 (s, 1H), 7.02 (d, J = 8.2 Hz, 1H), 6.86 (d, J = 8.2 Hz, 1H), 1.89 (s, 3H), 1.81 (s, 3H). |
| | (S)-5-(3-hydroxy-2,6- | | |
| | dimethylphenyl)-2-(1H-pyrazolo[4,3- | | |
| | b]pyridin-6-yl)-1H-pyrrolo[2,3- | | |
| | b]pyridine-4-carbonitrile | | |
| 125 | 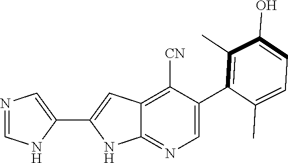 | 330.2 | (400 MHz, DMSO-d₆) δ 12.64 (s, 2H), 9.36 (s, 1H), 8.01 (s, 1H), 7.87 (s, 2H), 7.00 (d, J = 8.2 Hz, 1H), 6.84 (d, J = 8.2 Hz, 2H), 1.88 (s, 3H), 1.80 (s, 3H). |
| | (S)-5-(3-hydroxy-2,6- | | |
| | dimethylphenyl)-2-(1H-imidazol-5- | | |
| | yl)-1H-pyrrolo[2,3-b]pyridine-4- | | |
| | carbonitrile | | |
| 129 | 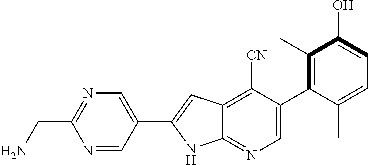 | 370.9 | ¹H NMR (400 MHz, DMSO- d₆) δ 9.50 (s, 2H), 8.37 (s, 1H), 8.23 (s, 1H), 7.51 (s, 1H), 7.02 (d, J = 8.2 Hz, 1H), 6.87 (d, J = 8.2 Hz, 1H), 4.16 (s, 2H), 1.88 (s, 3H), 1.80 (s, 3H). |
| | (S)-2-(2-(aminomethyl)pyrimidin-5- | | |
| | yl)-5-(3-hydroxy-2,6- | | |
| | dimethylphenyl)-1H-pyrrolo[2,3- | | |
| | b]pyridine-4-carbonitrile | | |
| 130 | 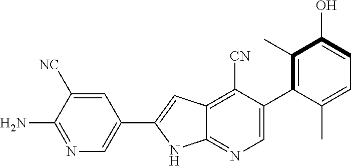 | 381.1 | ¹H NMR (400 MHz, DMSO- d₆) δ 12.71 (s, 1H), 9.33 (s, 1H), 8.95 (d, J = 2.5 Hz, 1H), 8.59 (d, J = 2.5 Hz, 1H), 8.08 (s, 1H), 7.38 (s, 2H), 7.17 (s, 1H), 7.00 (d, J = 8.2 Hz, 1H), 6.83 (d, J = 8.2 Hz, 1H), 1.86 (s, 3H), 1.79 (s, 3H). |
| | (S)-2-(6-amino-5-cyanopyridin-3-yl)- | | |
| | 5-(3-hydroxy-2,6-dimethylphenyl)- | | |
| | 1H-pyrrolo[2,3-b]pyridine-4- | | |
| | carbonitrile | | |
| 131 | 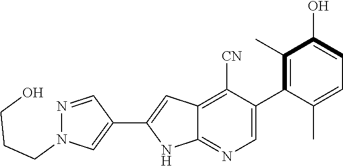 | | |
| | (S)-5-(3-hydroxy-2,6- | | |
| | dimethylphenyl)-2-(1-(3- | | |
| | hydroxypropyl)-1H-pyrazol-4-yl)- | | |
| | 1H-pyrrolo[2,3-b]pyridine-4- | | |
| | carbonitrile | | |
| 132 | 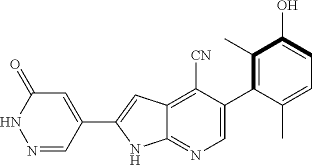 | 358.1 | ¹H NMR (400 MHz, DMSO- d₆) δ 13.04 (s, 2H), 9.34 (s, 1H), 8.63 (d, J = 2.0 Hz, 1H), 8.29 (s, 1H), 7.61 (s, 1H), 7.49 (d, J = 1.9 Hz, 1H), 7.02 (d, J = 8.2 Hz, 1H), 6.85 (d, J = 8.2 Hz, 1H), 1.87 (s, 3H), 1.79 (s, 3H). |
| | (S)-5-(3-hydroxy-2,6- | | |
| | dimethylphenyl)-2-(6-oxo-1,6- | | |
| | dihydropyridazin-4-yl)-1H- | | |
| | pyrrolo[2,3-b]pyridine-4-carbonitrile | | |

### Example 126: Preparation of (S)-2-(2-aminothiazol-5-yl)-5-(3-hydroxy-2,6-dimethylphenyl)-1H-pyrrolo[2,3-b]pyridine-4-carbonitrile

The title compound was prepared according to the following synthetic procedures.

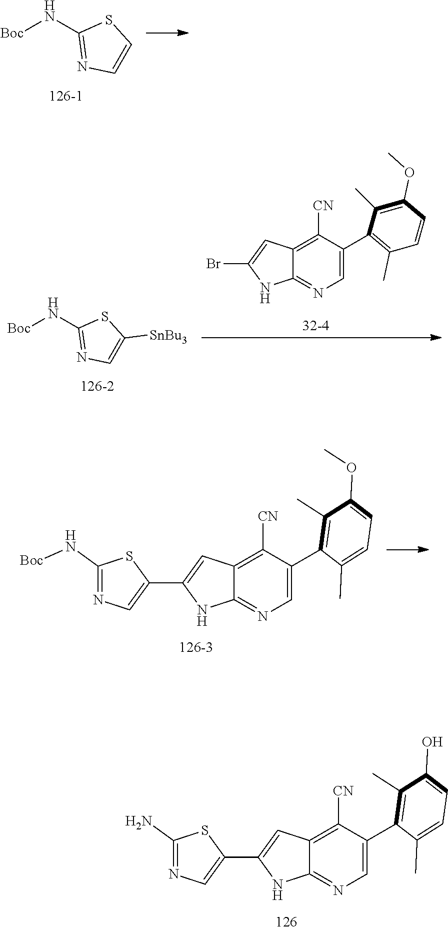

### Step 1: Preparation of Compound 126-2

To a solution of compound 126-1 (300.0 mg, 1.498 mmol) in THF (6 mL) was added n-BuLi (1.26 mL, 3.151 mmol) at −78° C. The mixture was stirred at −78° C. for 0.5 h. Then (n-Bu)₃SnCl (536.4 mg, 1.648 mmol) was added and the mixture was stirred at −78° C. for 0.5 h, the reaction was allowed to warm to rt and stirred for 3 h. The mixture was diluted with water (40 mL), which was extracted with EA (40 mL×3). The combined organic layer was washed with brine (40 mL), dried over Na₂SO₄, filtered, and concentrated to dryness. The residue was purified by silica gel column chromatography to afford compound 126-2. ¹H NMR: (400 MHz, CDCl₃) δ 12.27 (s, 1H), 7.23 (d, J=7.3 Hz, 1H), 1.52 (s, 9H), 1.49-1.43 (m, 6H), 1.26 (dd, J=14.7, 7.3 Hz, 6H), 1.07-0.98 (m, 6H), 0.82 (t, J=7.3 Hz, 9H).

### Step 2: Preparation of Compound 126-3

To a solution of compound 126-2 (190.0 mg, 0.388 mmol) in dioxane (5 mL) were added compound 32-4 (69.1 mg, 0.194 mmol), LiCl (16.4 mg, 0.387 mmol) and Pd(PPh₃)₄ (44.8 mg, 0.0388 mmol). The mixture was stirred at 100° C. for 5 h. The mixture was diluted with water (30 mL), which was extracted with EA (30 mL×3). The combined organic layers were washed with brine (30 mL), dried over Na₂SO₄, filtered, and concentrated to dryness. The residue was purified by prep-TLC to afford compound 126-3. LCMS: 476.3 [M+H]⁺.

### Step 3: Preparation of Example 126

To a solution of compound 126-3 (60.0 mg, 0.126 mmol) in DCM (4 mL) was added BBr₃ (315.7 mg, 1.26 mmol) at 0° C. The mixture was stirred at rt for 1 h. The reaction was quenched with MeOH (10 mL). The mixture was concentrated to dryness and purified by prep-HPLC to afford example 126. LCMS: 362.1 [M+H]⁺; ¹H NMR: (400 MHz, DMSO-d₆) δ 12.69 (s, 1H), 9.33 (s, 11H), 8.00 (s, 1H), 7.74 (s, 1H), 7.54 (s, 2H), 6.99 (d, J=8.2 Hz, 11H), 6.83 (d, J=8.2 Hz, 1H), 6.52 (s, 1H), 1.86 (s, 3H), 1.78 (s, 3H).

The following examples 132 were prepared according to the methods described in examples 126 with analogous starting materials.

| | | LCMS | |
|---|---|---|---|
| Example | Structure | [M + H]⁺ | ¹H NMR |
| 132 | 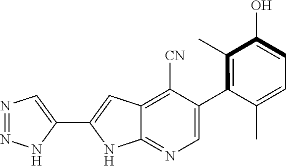 | 371.9 | ¹H NMR: (400 MHz, DMSO-d₆) δ 15.50 (d, J = 115.6 Hz, 1H), 13.03 (d, J = 21.1 Hz, 1H), 9.34 (s, 1H), 8.58 (d, J = 100.5 Hz, 1H), 8.14 (d, J = 13.9 Hz, 1H), 7.10 (s, 1H), 7.01 (d, J = 8.3 Hz, 1H), 6.84 (d, J = 8.2 Hz, 1H), 1.87 (s, 3H), 1.79 (s, 3H). |
| | 5-(3-hydroxy-2,6-dimethylphenyl)-2- | | |
| | (1H-1,2,3-triazol-5-yl)-1H- | | |
| | pyrrolo[2,3-b]pyridine-4-carbonitrile | | |

### Example 127: Preparation of (S)-2-(3-amino-1,2,4-triazin-6-yl)-5-(3-hydroxy-2,6-dimethylphenyl)-1H-pyrrolo[2,3-b]pyridine-4-carbonitrile

The title compound was prepared according to the following synthetic procedures.

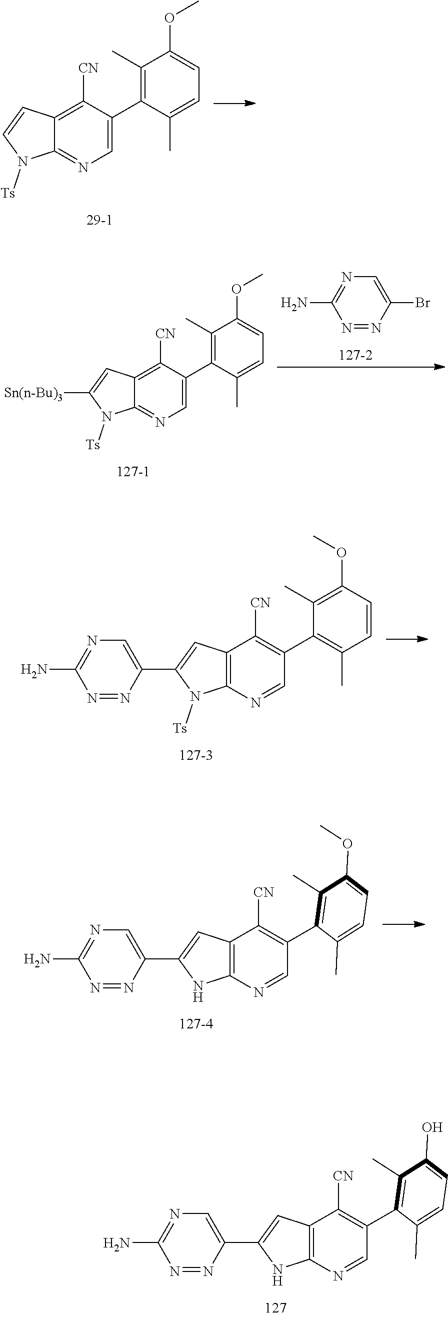

### Step 1: Preparation of Compound 127-1

To a solution of compound 29-1 (3.0, 6.952 mmol) in THF (75.0 mL) was added LDA (7.0 mL, 13.905 mmol) in drops at −78 [symbol] under N₂. The mixture was stirred at −78 [symbol] for 30 min. Then (n-Bu)₃SnCl (4.53 g, 13.905 mmol) was added. The mixture was stirred at −78 [symbol] for 1 h and then warmed to [symbol] rt for 0.5 h. The mixture was quenched by st.aq NH₄Cl (30 mL), extracted with EA (40 mL×3). Combined organic layers were washed with brine, dried over Na₂SO₄, filtered and concentrated. The residue was purified by silica gel column chromatography to afford compound 127-1 (1.28 g, 1.776 mmol, 25.5%). ¹H NMR: (400 MHz, CDCl₃) δ 8.18 (s, 1H), 8.07 (d, J=8.3 Hz, 2H), 7.30 (d, J=8.3 Hz, 2H), 7.10 (d, J=8.4 Hz, 1H), 6.90-6.83 (m, 2H), 3.85 (s, 3H), 2.41 (s, 3H), 1.90 (s, 3H), 1.83 (s, 3H), 1.70-1.56 (m, 6H), 1.41 (dd, J=14.6, 7.3 Hz, 6H), 1.35-1.25 (m, 6H), 0.94 (t, J=7.3 Hz, 9H).

### Step 2: Preparation of Compound 127-3

To a solution of compound 127-1 (400.0 mg, 0.555 mmol) in toluene (6.0 mL) were added compound 127-2 (97.1 mg, 0.555 mmol), Pd(PPh₃)₄ (64.1 mg, 0.056 mmol) and CuI (21.1 mg, 0.111 mmol). The mixture was stirred at 120° C. under N₂ for 1 h. The reaction mixture was diluted with water (20 mL), which was extracted with EA (30 mL×3). The combined organic layers were washed with brine (30 mL), dried over Na₂SO₄, filtered, and concentrated to dryness. The residue was purified by silica gel column chromatography to afford compound 127-3. LCMS: 526.3 [M+H]⁺.

### Step 3: Preparation of Compound 127-4

A mixture of compound 127-3 (172.0 mg, 0.327 mmol) and NaOH (130.9 mg, 3.272 mmol) in EtOH (6.0 mL) was stirred at 60° C. under N₂ for 1 h. The pH of the reaction mixture was adjusted to 7 with HCl (1 M in water) at 0 [symbol] . The mixture was extracted with EA (20 mL×3). The combined organic layer was washed with brine (90 mL), dried over Na₂SO₄, filtered, and concentrated to dryness. The residue was purified by silica gel column chromatography and further purified by SFC to afford compound 127-4. LCMS: 413.1 [M+H+CH₃CN]⁺.

### Step 4: Preparation of Example 127

To a solution of compound 127-4 (30.0 mg, 0.081 mmol) in DCM (3.0 mL) was added BBr₃ (0.04 mL, 0.404 mmol) at 0 [symbol] . The mixture was stirred at rt for 1 h. The reaction mixture was carefully added into MeOH (2 mL) while stirring and was diluted with water (10 mL), the pH of which was adjusted to 8 with NaHCO₃. The mixture was extracted with DCM (30 mL) and EA (20 mL×2). The combined organic layer was dried over Na₂SO₄, filtered, and concentrated to dryness. The residue was purified by prep-HPLC to afford example 127. LCMS: 358.0 [M+H]⁺; ¹H NMR: (400 MHz, DMSO-d₆) δ 9.35 (s, 1H), 9.08 (s, 1H), 8.16 (s, 1H), 7.66 (s, 2H), 7.34 (s, 1H), 7.01 (d, J=8.2 Hz, 1H), 6.85 (d, J=8.2 Hz, 1H), 1.88 (s, 3H), 1.80 (s, 3H).

### Example 128: Preparation of 3-(6-(2-((1S,4S)-2-oxa-5-azabicyclo[2.2.1]heptan-5-yl)pyrimidin-5-yl)-5H-pyrrolo[2,3-b]pyrazin-2-yl)-2,4-dimethylphenol

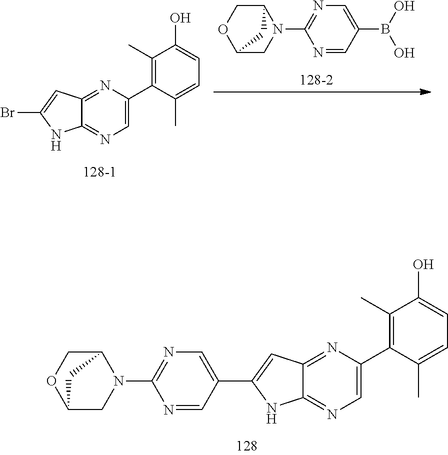 [SMILES: Cc1ccc(O)c(C)c1-c1cnc2[nH]c(Br)cc2n1 ; OB(O)c1cnc(N2C[C@@H]3C[C@H]2CO3)nc1 ; Cc1ccc(O)c(C)c1-c1cnc2[nH]c(-c3cnc(N4C[C@@H]5C[C@H]4CO5)nc3)cc2n1]

### Step 1: Preparation of Example 128

A mixture of compound 128-1 (165.0 mg, 0.746 mmol), compound 128-2 (274.3 mg, 1.119 mmol), K₃PO₄ (190.1 mg, 0.896 mmol) and Pd(dtbpf)Cl₂ (19.5 mg, 0.030 mmol) in dioxane-water (3 mL) was stirred at 90° C. under N₂ for 3 h. The reaction mixture was diluted with water (20 mL), which was extracted with EA (30 mL×3). The combined organic layer was washed with brine (30 mL), dried over Na₂SO₄, filtered, and concentrated to dryness. The residue was purified by prep-HPLC to afford example 128. LCMS: 415.2 [M+H]⁺; ¹H NMR: (400 MHz, DMSO-d₆) δ 12.49 (s, 1H), 9.21 (s, 1H), 9.00 (s, 2H), 8.02 (s, 1H), 7.06 (s, 1H), 6.94 (d, J=8.2 Hz, 1H), 6.79 (d, J=8.1 Hz, 1H), 5.04 (s, 1H), 4.72 (s, 1H), 3.84 (d, J=7.2 Hz, 1H), 3.72 (d, J=7.3 Hz, 1H), 3.56 (d, J=10.6 Hz, 1H), 3.47 (d, J=10.9 Hz, 1H), 1.98 (d, J=8.5 Hz, 1H), 1.92 (s, 1H), 1.88 (s, 3H), 1.80 (s, 3H).

### Example 133: Preparation of (S)-2-(4-amino-1H-pyrazol-1-yl)-5-(3-hydroxy-2,6-dimethylphenyl)-1H-pyrrolo[2,3-b]pyridine-4-carbonitrile

The title compound was prepared according to the following synthetic procedures.

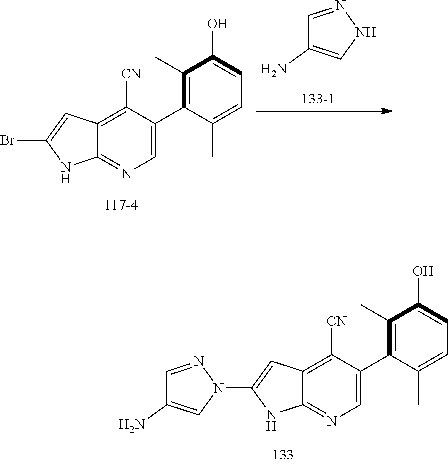

A mixture of 2-bromo-5-(3-hydroxy-2,6-dimethylphenyl)-1H-pyrrolo[2,3-b]pyridine-4-carbonitrile (70 mg, 0.205 mmol), 1H-pyrazol-4-amine (34.0 mg, 0.409 mmol), methyl[(1S,2S)-2-(methylamino)cyclohexyl]amine (5.8 mg, 0.041 mmol), t-BuONa (59.0 mg, 0.614 mmol) and CuI (7.8 mg, 0.041 mmol) in DMF (0.5 mL) was heated to 100 [symbol] for 16 h under N₂ atmosphere. The mixture was purified by Pre-HPLC to afford compound 133. LCMS: 345.1 [M+H]⁺; ¹H NMR (400 MHz, DMSO-d₆) δ 13.71-12.17 (br s, 1H), 9.29 (s, 1H), 7.96 (s, 1H), 7.84 (s, 1H), 7.44 (s, 1H), 6.99 (d, J=8.1 Hz, 1H), 6.83 (d, J=8.0 Hz, 1H), 6.61 (s, 1H), 4.41 (s, 1H), 1.87 (s, 3H), 1.79 (s, 3H).

### Example 135: Preparation of 5-(3-hydroxy-2,6-dimethylphenyl)-2-(4H-1,2,4-triazol-3-yl)-1H-pyrrolo[2,3-b]pyridine-4-carbonitrile

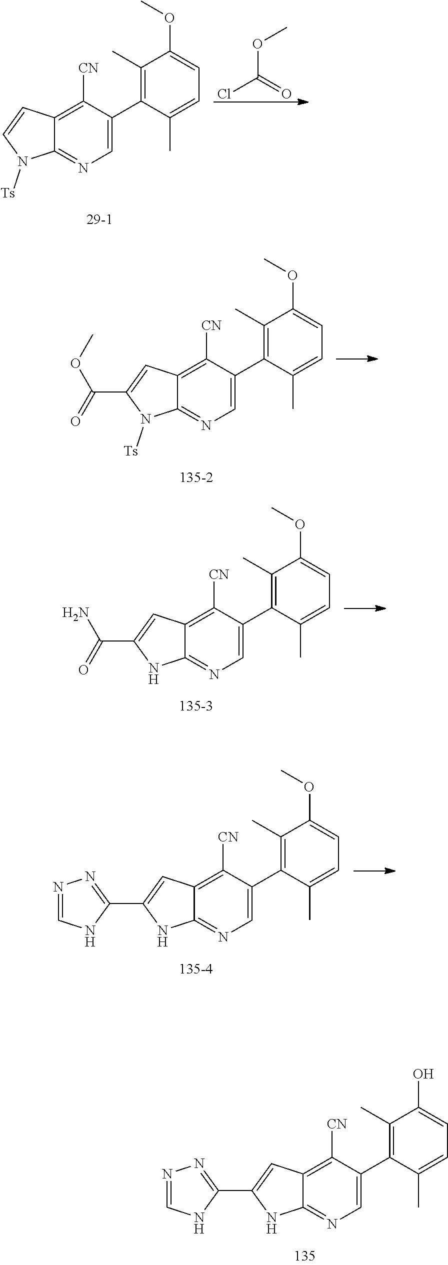

### Step 1: Preparation of Compound 135-2

To a solution of Compound 29-1 (2000 mg, 4.635 mmol) in THF (50 mL) was added LDA (4.63 mL, 9.270 mmol) at −78 [symbol] . The mixture was stirred for 0.5 h before adding a solution of methyl chloroformate (876.0 mg, 9.27 mmol) in THF (15 mL). The mixture was stirred at −78 [symbol] for 2 h, quenched with NH₄Cl. aq (50 mL), diluted with water (200 mL), and extracted with EA (100 mL×3). The combined organic phase was washed with brine (150 mL×2), dried over Na₂SO₄, filtered, and concentrated. The residue was purified by silica gel column chromatography to afford Compound 135-2 as a white solid (1.0 g, 44.1% yield). LCMS: 489.8 [M+H]⁺.

### Step 2: Preparation of Compound 135-3

A mixture of Compound 135-2 (200 mg, 0.409 mmol) in ammonia (7.0 M in MeOH, 10 mL, 70.0 mmol) was stirred at 65 [symbol] for 10 h. The mixture was concentrated to afford Compound 135-3. LCMS: 321.2 [M+H]⁺.

### Step 3: Preparation of Compound 135-4

A solution of Compound 135-3 (100 mg, 0.312 mmol) in N,N-dimethylformamide dimethyl acetal (5 mL) was stirred at 90 [symbol] for 1 h. The mixture was concentrated and dissolved in EtOH (10 mL) to fsi afford P1. To a solution of hydrazinium hydroxide (1 mL, 20.559 mmol) in AcOH (3 mL) in 0 [symbol] was added P1. The mixture was stirred at rt for 1 h, and concentrated. The residue was purified by silica gel column chromatography to afford Compound 135-4. LCMS: 345.1 [M+H]⁺.

### Step 3: Preparation of Example 135

A mixture of Compound 135-4 (15 mg, 0.044 mmol) in DCM (5 mL) was added boron tribromide (0.25 mL) at 0 [symbol] . The mixture was stirred for 1 h, quenched with NH₃/MeOH (7 M) at 0 [symbol] and concentrated. The residue was purified by Pre-TLC (MeOH:DCM-1:10) to afford Example 135. LCMS: 331.1 [M+H]⁺; ¹H NMR (400 MHz, DMSO-d₆) δ 14.52 (s, 1H), 13.14 (s, 1H), 9.35 (s, 1H), 8.72 (s, 1H), 8.19 (s, 1H), 7.20-6.94 (m, 2H), 6.85 (d, J=8.2 Hz, 1H), 1.87 (s, 3H), 1.80 (s, 3H).

### Example A: PKMYT1 HTRF Assay

Compound serial dilution is performed by Echo, and the final concentrations vary from 10 μM to 0.5 nM. This was filled by the addition of 5 μL/well of Enzyme solution to the assay plate containing the compound. The plate was centrifuged at 1000 rpm for 1 minute, and incubate 15 minutes at 25 [symbol] . Then 5 μL/well of tracer solution (Tracer 178) was added to initiate the reaction, and incubate for 60 minutes at 25° C. Next 5 μL GST-Tb was added into the assay plate, the plate was centrifuged at 1000 rpm for 1 minute, and incubate for 15 minutes at 25 [symbol] . The assay plate was read on Envision.

The data for Example A is shown in Table 3.

TABLE 3

| Ex. | MYT1 HTRF IC₅₀ (nM) |
|---|---|
| 1 | C |
| 2 | D |
| 3 | C |
| 4 | C |
| 5 | B |
| 6 | C |
| 7 | B |
| 8 | B |
| 9 | B |
| 10 | A |
| 11 | A |
| 12 | B |
| 13 | B |
| 14 | A |
| 15 | B |
| 16 | A |
| 17 | A |
| 18 | A |
| 19 | B |
| 20 | A |
| 21 | A |
| 22 | C |
| 23 | A |
| 24 | A |
| 25 | A |
| 26 | A |
| 27 | B |
| 29 | C |
| 30 | C |
| 31 | A |
| 32 | A |
| 33 | A |
| 34 | C |
| 35 | A |
| 36 | B |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | B |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | B |
| 78 | C |
| 79 | B |
| 80 | A |
| 81 | C |
| 82 | A |
| 83 | A |
| 84 | A |
| 85 | A |
| 86 | A |
| 87 | A |
| 88 | B |
| 89 | A |
| 90 | C |
| 91 | B |
| 92 | A |
| 93 | A |
| 94 | A |
| 95 | A |
| 96 | B |
| 97 | A |
| 98 | A |
| 99 | A |
| 100 | A |
| 101 | A |
| 102 | A |
| 103 | A |
| 104 | A |
| 105 | A |
| 106 | A |
| 107 | A |
| 108 | B |
| 109 | A |
| 110 | A |
| 111 | A |
| 112 | B |
| 113 | C |
| 114 | C |
| 115 | C |
| 116 | A |
| 117 | A |
| 118 | A |
| 119 | A |
| 120 | A |
| 121 | A |
| 122 | A |
| 123 | A |
| 124 | A |
| 125 | A |
| 126 | A |
| 127 | A |
| 128 | A |
| 129 | A |
| 130 | A |
| 131 | A |
| 132 | B |
| 133 | A |
| 134 | A |
| 135 | A |

MYT1 HTRF IC₅₀ (nM): 0<A≤10; 10<B≤50; 50<C≤500; 500<D≤5000; 5000<E

### Example B: WEE1 ADP-Glo Assay

Compound serial dilution is performed by Echo, and the final concentrations vary from 10 μM to 0.5 nM. This was filled by the addition of 5 μL/well of Enzyme solution to the assay plate containing the compound. The plate was centrifuged at 1000 rpm for 1 minute, and incubate 15 minutes at 25 [symbol] , Then 5 μL/well of substrate solution was added to initiate the reaction, and incubate for 60 minutes at 25° C. Next 10 μL kinase detection reagent was added into the assay plate, the plate was centrifuged at 1000 rpm for 1 minute, and incubate for 60 minutes at 25 [symbol] . The assay plate was read on Envision for US LUM as RLU.

What is claimed is:

1. A compound of Formula (I), or a pharmaceutically acceptable salt thereof: 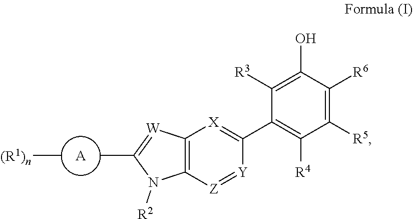
wherein:
Ring A is cycloalkyl, heterocycloalkyl, aryl, or heteroaryl;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(=O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{1a};
or two R¹ on the same atom are taken together to form an oxo;
each R^{1a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(=O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{1a} on the same atom are taken together to form an oxo;
n is 0-6;
R² is hydrogen, C₁-C₆alkyl, C₁-C₆haloalkyl, C₁-C₆hydroxyalkyl, C₁-C₅aminoalkyl, C₁-C₆heteroalkyl, cycloalkyl, or heterocycloalkyl;
W is N or CR^{W};
R^{W} is hydrogen, halogen, —CN, —OH, —OR^{a}, —SH, —SR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Wa};
each R^{Wa} is independently halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Wa} on the same atom are taken together to form an oxo;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R³ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁴ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

2. The compound of claim 1, or a pharmaceutically acceptable salt thereof, wherein X is N.

3. The compound of claim 1, or a pharmaceutically acceptable salt thereof, wherein X is CR^{X}.

4. The compound of any one of claims 1-3, or a pharmaceutically acceptable salt thereof, wherein Y is N.

5. The compound of any one of claims 1-3, or a pharmaceutically acceptable salt thereof, wherein Y is CR^{Y}.

6. The compound of any one of claims 1-5, or a pharmaceutically acceptable salt thereof, wherein W is N.

7. The compound of any one of claims 1-5, or a pharmaceutically acceptable salt thereof, wherein W is CR^{W}.

8. The compound of any one of claims 1-7, or a pharmaceutically acceptable salt thereof, wherein Z is N.

9. The compound of any one of claims 1-7, or a pharmaceutically acceptable salt thereof, wherein Z is CR^{Z}.

10. The compound of claim 1, or a pharmaceutically acceptable salt thereof, wherein the compound is of Formula (Ia):

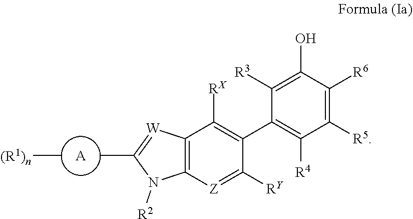

11. The compound of claim 1, or a pharmaceutically acceptable salt thereof, wherein the compound is of Formula (Ib):

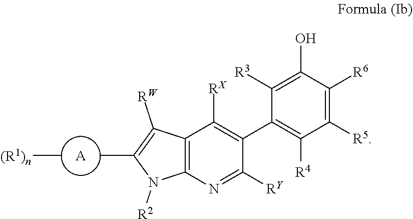

12. The compound of claim 1, or a pharmaceutically acceptable salt thereof, wherein the compound is of Formula (Ic):

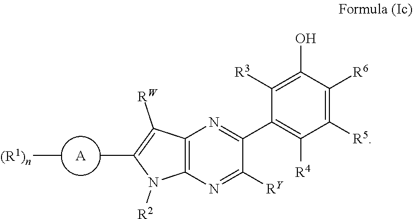

13. The compound of any one of claims 1-12, or a pharmaceutically acceptable salt thereof, wherein R³ is halogen, C₁-C₆alkyl, or C₁-C₆haloalkyl.

14. The compound of any one of claims 1-13, or a pharmaceutically acceptable salt thereof, wherein R³ is C₁-C₅alkyl.

15. The compound of any one of claims 1-14, or a pharmaceutically acceptable salt thereof, wherein R⁴ is halogen, C₁-C₆alkyl, or C₁-C₆haloalkyl.

16. The compound of any one of claims 1-15, or a pharmaceutically acceptable salt thereof, wherein R⁴ is C₁-C₆alkyl.

17. The compound of any one of claims 1-16, or a pharmaceutically acceptable salt thereof, wherein R⁵ is hydrogen.

18. The compound of any one of claims 1-17, or a pharmaceutically acceptable salt thereof, wherein R⁶ is hydrogen.

19. The compound of any one of claims 1-18, or a pharmaceutically acceptable salt thereof, wherein R² is hydrogen or C₁-C₆alkyl.

20. The compound of any one of claims 1-19, or a pharmaceutically acceptable salt thereof, wherein R² is hydrogen.

21. The compound of any one of claims 1-20, or a pharmaceutically acceptable salt thereof, wherein R^{X} is hydrogen, halogen, C₁-C₆alkyl, or C₁-C₆haloalkyl.

22. The compound of any one of claims 1-21, or a pharmaceutically acceptable salt thereof, wherein R^{X} is hydrogen, halogen, or C₁-C₆alkyl.

23. The compound of any one of claims 1-22, or a pharmaceutically acceptable salt thereof, wherein R^{X} is hydrogen or halogen.

24. The compound of any one of claims 1-23, or a pharmaceutically acceptable salt thereof, wherein R^{X} is hydrogen.

25. The compound of any one of claims 1-24, or a pharmaceutically acceptable salt thereof, wherein R^{Y} is hydrogen, halogen, C₁-C₆alkyl, or C₁-C₆haloalkyl.

26. The compound of any one of claims 1-25, or a pharmaceutically acceptable salt thereof, wherein R^{Y} is hydrogen.

27. The compound of any one of claims 1-26, or a pharmaceutically acceptable salt thereof, wherein R^{Z} is hydrogen, halogen, or C₁-C₆alkyl.

28. The compound of any one of claims 1-27, or a pharmaceutically acceptable salt thereof, wherein R^{Z} is hydrogen or halogen.

29. The compound of any one of claims 1-28, or a pharmaceutically acceptable salt thereof, wherein R^{Z} is hydrogen.

30. The compound of any one of claims 1-29, or a pharmaceutically acceptable salt thereof, wherein R^{W} is hydrogen, halogen, —OH, —OR^{a}, —NR^{c}R^{d}, C₁-C₆alkyl, C₁-C₆haloalkyl, cycloalkyl, heterocycloalkyl, aryl, or heteroaryl; wherein the alkyl, cycloalkyl, heterocycloalkyl, aryl, and heteroaryl is optionally substituted with one or more R.

31. The compound of any one of claims 1-30, or a pharmaceutically acceptable salt thereof, wherein R^{W} is hydrogen, halogen, —OR^{a}, C₁-C₆alkyl, C₁-C₆haloalkyl, cycloalkyl, or heterocycloalkyl; wherein the alkyl, cycloalkyl, and heterocycloalkyl is optionally substituted with one or more R.

32. The compound of any one of claims 1-31, or a pharmaceutically acceptable salt thereof, wherein R^{W} is hydrogen, —OR^{a}, C₁-C₆alkyl, or heterocycloalkyl; wherein the alkyl and heterocycloalkyl is optionally substituted with one or more R.

33. The compound of any one of claims 1-32, or a pharmaceutically acceptable salt thereof, wherein R^{W} is hydrogen.

34. The compound of any one of claims 1-32, or a pharmaceutically acceptable salt thereof, wherein R^{W} is heterocycloalkyl optionally substituted with one or more R.

35. The compound of any one of claims 1-34, or a pharmaceutically acceptable salt thereof, wherein Ring A is cycloalkyl or heterocycloalkyl.

36. The compound of any one of claims 1-35, or a pharmaceutically acceptable salt thereof, wherein Ring A is heterocycloalkyl.

37. The compound of any one of claims 1-36, or a pharmaceutically acceptable salt thereof, wherein Ring A is 5- or 6-membered heterocycloalkyl.

38. The compound of any one of claims 1-37, or a pharmaceutically acceptable salt thereof, wherein Ring A is tetrahydropyridinyl.

39. The compound of any one of claims 1-34, or a pharmaceutically acceptable salt thereof, wherein Ring A is aryl or heteroaryl.

40. The compound of any one of claims 1-34 or 39, or a pharmaceutically acceptable salt thereof, wherein Ring A is heteroaryl.

41. The compound of any one of claims 1-34 or 39 or 40, or a pharmaceutically acceptable salt thereof, wherein Ring A is 5- or 6-membered heteroaryl.

42. The compound of any one of claims 1-34 or 39 or 40, or a pharmaceutically acceptable salt thereof, wherein Ring A is monocyclic heteroaryl.

43. The compound of any one of claims 1-34 or 39 or 40, or a pharmaceutically acceptable salt thereof, wherein Ring A is bicyclic heteroaryl.

44. The compound of any one of claims 1-34 or 39 or 40, or a pharmaceutically acceptable salt thereof, wherein Ring A is pyridinyl.

45. The compound of any one of claims 1-44, or a pharmaceutically acceptable salt thereof, wherein each R¹ is independently halogen, —CN, —OH, —OR, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently optionally independently substituted with one or more R^{1a}.

46. The compound of any one of claims 1-45, or a pharmaceutically acceptable salt thereof, wherein each R¹ is independently halogen, —OR^{a}, —NR^{c}R^{d}, —C(═O)R^{a}, C₁-C₆alkyl, C₁-C₆haloalkyl, C₁-C₆hydroxyalkyl, cycloalkyl, or heterocycloalkyl; wherein each alkyl, cycloalkyl, and heterocycloalkyl is independently optionally independently substituted with one or more R^{1a}.

47. The compound of any one of claims 1-46, or a pharmaceutically acceptable salt thereof, wherein each R¹ is independently halogen, —OR^{a}, —NR^{c}R^{d}, —C(═O)R^{a}, C₁-C₆alkyl, C₁-C₆hydroxyalkyl, or heterocycloalkyl; wherein each alkyl, and heterocycloalkyl is independently optionally independently substituted with one or more R^{1a}.

48. The compound of any one of claims 1-47, or a pharmaceutically acceptable salt thereof, wherein each R^{1a} is independently halogen, —CN, —OH, —OR^{a}, —NR^{c}R^{d}, —C(═O)R, —C(═O)OR^{b}, —C(═O)NR^{c}R^{d}, C₁-C₆alkyl, C₁-C₆haloalkyl, cycloalkyl, or heterocycloalkyl; wherein each alkyl, cycloalkyl, heterocycloalkyl is independently optionally substituted with one or more R.

49. The compound of any one of claims 1-48, or a pharmaceutically acceptable salt thereof, wherein each R^{1a} is independently halogen, C₁-C₆alkyl, or C₁-C₆haloalkyl; wherein each alkyl is independently optionally substituted with one or more R.

50. The compound of any one of claims 1-49, or a pharmaceutically acceptable salt thereof, wherein each R^{1a} is independently halogen, C₁-C₆alkyl, or C₁-C₆haloalkyl.

51. The compound of any one of claims 1-50, or a pharmaceutically acceptable salt thereof, wherein n is 0-2.

52. The compound of any one of claims 1-51, or a pharmaceutically acceptable salt thereof, wherein n is 1 or 2.

53. The compound of any one of claims 1-52, or a pharmaceutically acceptable salt thereof, wherein n is 1.

54. The compound of claim 1, or a pharmaceutically acceptable salt thereof, wherein the compound is selected from a compound found in table 1 or table 2.

55. A pharmaceutical composition comprising the compound of any one of claims 1-54, or a pharmaceutically acceptable salt thereof, and a pharmaceutically acceptable excipient.

56. A method of treating cancer in a subject in need thereof, the method comprising administering to the subject the compound of any one of claims 1-54, or a pharmaceutically acceptable salt thereof.

57. A method of modulating PKMYT1 in a subject, the method comprising administering to the subject the compound of any one of claims 1-54, or a pharmaceutically acceptable salt thereof.

58. A method of inhibiting PKMYT1 in a subject, the method comprising administering to the subject the compound of any one of claims 1-54, or a pharmaceutically acceptable salt thereof.

59. The method of claim 57 or 58, wherein the subject has cancer.

60. The method of claim 56, wherein the cancer depends on the activity of PKMYT1.

61. The method of any one of claims 56 or 60, wherein the cancer overexpresses CCNE1.

62. The method of any one of claims 56 or 60 or 61, wherein the cancer has an inactivating mutation in the FBXW7 gene.

63. The method of any one of claims 56 or 60-62, wherein the cancer is a solid tumor.

64. The method of any one of claims 56 or 60-63, wherein the cancer is breast cancer, colorectal cancer, endometrial cancer, esophageal cancer, glioblastoma, hepatocellular carcinoma, lung cancer, neuroblastoma, ovarian cancer, prostate cancer, stomach cancer, or uterine cancer.
